(19) United States
(12) Patent Application Publication
(10) Pub. No.: US 2006/0122210 A1
(43) Pub. Date: Jun. 8, 2006

(54) Inhibitors of 11-beta hydroxyl steroid dehydrogenase type I and methods of using the same

(76) Inventors: Wenqing Yao, Kennett Square, PA (US); Chunghong He, Boothwyn, PA (US); Jingcong Zhuo, Boothwyn, PA (US)

Correspondence Address: COZEN O' CONNOR, P.C., 1900 MARKET STREET, PHILADELPHIA, PA, 19103-3508, US

(21) Appl. No.: 11/281,648

(22) Filed: Nov. 17, 2005

Related U.S. Application Data
(60) Provisional application No. 60/628,933, filed on Nov. 18, 2004.

(51) Int. Cl. A61K 31/4747; C07D 471/10

(52) U.S. Cl. 514/278; 546/16

(57) ABSTRACT

The present invention relates to inhibitors of 11-β hydroxyl steroid dehydrogenase type 1, antagonists of the mineralocorticoid receptor (MR), and pharmaceutical compositions thereof. The compounds of the invention can be useful in the treatment of various diseases associated with expression or activity of 11-β hydroxyl steroid dehydrogenase type 1 and/or diseases associated with aldosterone excess.

### CROSS-REFERENCE TO RELATED APPLICATIONS

This application claims the benefit of U.S. Ser. No. 60/628,933, filed Nov. 18, 2004, the disclosure of which is incorporated herein by reference in its entirety.

### FIELD OF THE INVENTION

The present invention relates to modulators of 11-β hydroxyl steroid dehydrogenase type 1 (11βHSD1) and/or mineralocorticoid receptor (MR), compositions thereof and methods of using the same.

### BACKGROUND OF THE INVENTION

Glucocorticoids are steroid hormones that regulate fat metabolism, function and distribution. In vertebrates, glucocorticoids also have profound and diverse physiological effects on development, neurobiology, inflammation, blood pressure, metabolism and programmed cell death. In humans, the primary endogenously-produced glucocorticoid is cortisol. Cortisol is synthesized in the zona fasciculate of the adrenal cortex under the control of a short-term neuroendocrine feedback circuit called the hypothalamic-pituitary-adrenal (HPA) axis. Adrenal production of cortisol proceeds under the control of adrenocorticotrophic hormone (ACTH), a factor produced and secreted by the anterior pituitary. Production of ACTH in the anterior pituitary is itself highly regulated, driven by corticotropin releasing hormone (CRH) produced by the paraventricular nucleus of the hypothalamus. The HPA axis maintains circulating cortisol concentrations within restricted limits, with forward drive at the diurnal maximum or during periods of stress, and is rapidly attenuated by a negative feedback loop resulting from the ability of cortisol to suppress ACTH production in the anterior pituitary and CRH production in the hypothalamus.

Aldosterone is another hormone produced by the adrenal cortex; aldosterone regulates sodium and potassium homeostasis. Fifty years ago, a role for aldosterone excess in human disease was reported in a description of the syndrome of primary aldosteronism (Conn, (1955), J. Lab. Clin. Med. 45: 6-17). It is now clear that elevated levels of aldosterone are associated with deleterious effects on the heart and kidneys, and are a major contributing factor to morbidity and mortality in both heart failure and hypertension.

Two members of the nuclear hormone receptor superfamily, glucocorticoid receptor (GR) and mineralocorticoid receptor (MR), mediate cortisol function in vivo, while the primary intracellular receptor for aldosterone is the MR. These receptors are also referred to as ‘ligand-dependent transcription factors,’ because their functionality is dependent on the receptor being bound to its ligand (for example, cortisol); upon ligand-binding these receptors directly modulate transcription via DNA-binding zinc finger domains and transcriptional activation domains.

Historically, the major determinants of glucocorticoid action were attributed to three primary factors: 1) circulating levels of glucocorticoid (driven primarily by the HPA axis), 2) protein binding of glucocorticoids in circulation, and 3) intracellular receptor density inside target tissues. Recently, a fourth determinant of glucocorticoid function was identified: tissue-specific pre-receptor metabolism by glucocorticoid-activating and -inactivating enzymes. These 11-beta-hydroxysteroid dehydrogenase (11-β-HSD) enzymes act as pre-receptor control enzymes that modulate activation of the GR and MR by regulation of glucocorticoid hormones. To date, two distinct isozymes of 11-beta-HSD have been cloned and characterized: 11βHSD1 (also known as 11-beta-HSD type 1, 11betaHSD1, HSD11B1, HDL, and HSD11L) and 11βHSD2. 11βHSD1 and 11βHSD2 catalyze the interconversion of hormonally active cortisol (corticosterone in rodents) and inactive cortisone (11-dehydrocorticosterone in rodents). 11βHSD1 is widely distributed in rat and human tissues; expression of the enzyme and corresponding mRNA have been detected in lung, testis, and most abundantly in liver and adipose tissue. 11βHSD1 catalyzes both 11-beta-dehydrogenation and the reverse 11-oxoreduction reaction, although 11βHSD1 acts predominantly as a NADPH-dependent oxoreductase in intact cells and tissues, catalyzing the activation of cortisol from inert cortisone (Low et al. (1994) J. Mol. Endocrin. 13: 167-174) and has been reported to regulate glucocorticoid access to the GR. Conversely, 11βHSD2 expression is found mainly in mineralocorticoid target tissues such as kidney, placenta, colon and salivary gland, acts as an NAD-dependent dehydrogenase catalyzing the inactivation of cortisol to cortisone (Albiston et al. (1994) Mol. Cell. Endocrin. 105: R11-R17), and has been found to protect the MR from glucocorticoid excess, such as high levels of receptor-active cortisol (Blum, et al., (2003) Prog. Nucl. Acid Res. Mol. Biol. 75:173-216).

In vitro, the MR binds cortisol and aldosterone with equal affinity. The tissue specificity of aldosterone activity, however, is conferred by the expression of 11βHSD2 (Funder et al. (1988), Science 242: 583-585). The inactivation of cortisol to cortisone by 11βHSD2 at the site of the MR enables aldosterone to bind to this receptor in vivo. The binding of aldosterone to the MR results in dissociation of the ligand-activated MR from a multiprotein complex containing chaperone proteins, translocation of the MR into the nucleus, and its binding to hormone response elements in regulatory regions of target gene promoters. Within the distal nephron of the kidney, induction of serum and glucocorticoid inducible kinase-1 (sgk-1) expression leads to the absorption of Na⁺ ions and water through the epithelial sodium channel, as well as potassium excretion with subsequent volume expansion and hypertension (Bhargava et al., (2001), Endo 142: 1587-1594).

In humans, elevated aldosterone concentrations are associated with endothelial dysfunction, myocardial infarction, left ventricular atrophy, and death. In attempts to modulate these ill effects, multiple intervention strategies have been adopted to control aldosterone overactivity and attenuate the resultant hypertension and its associated cardiovascular consequences. Inhibition of angiotensin-converting enzyme (ACE) and blockade of the angiotensin type 1 receptor (AT1R) are two strategies that directly impact the rennin-angiotensin-aldosterone system (RAAS). However, although ACE inhibition and AT1R antagonism initially reduce aldosterone concentrations, circulating concentrations of this hormone return to baseline levels with chronic therapy (known as ‘aldosterone escape’). Importantly, co-administration of the MR antagonist Spironolactone or Eplerenone directly blocks the deleterious effects of this escape mechanism and dramatically reduces patient mortality (Pitt et al., New England J. Med. (1999), 341: 709-719; Pitt et al., New England J. Med. (2003), 348: 1309-1321). Therefore, MR antagonism may be an important treatment strategy for many patients with hypertension and cardiovascular disease, particularly those hypertensive patients at risk for target-organ damage.

Mutations in either of the genes encoding the 11-beta-HSD enzymes are associated with human pathology. For example, 11βHSD2 is expressed in aldosterone-sensitive tissues such as the distal nephron, salivary gland, and colonic mucosa where its cortisol dehydrogenase activity serves to protect the intrinsically non-selective MR from illicit occupation by cortisol (Edwards et al. (1988) Lancet 2: 986-989). Individuals with mutations in 11βHSD2 are deficient in this cortisol-inactivation activity and, as a result, present with a syndrome of apparent mineralocorticoid excess (also referred to as ‘SAME’) characterized by hypertension, hypokalemia, and sodium retention (Wilson et al. (1998) Proc. Natl. Acad. Sci. 95: 10200-10205). Likewise, mutations in 11βHSD1, a primary regulator of tissue-specific glucocorticoid bioavailability, and in the gene encoding a co-localized NADPH-generating enzyme, hexose 6-phosphate dehydrogenase (H6PD), can result in cortisone reductase deficiency (CRD), in which activation of cortisone to cortisol does not occur, resulting in adrenocorticotropin-mediated androgen excess. CRD patients excrete virtually all glucocorticoids as cortisone metabolites (tetrahydrocortisone) with low or absent cortisol metabolites (tetrahydrocortisols). When challenged with oral cortisone, CRD patients exhibit abnormally low plasma cortisol concentrations. These individuals present with ACTH-mediated androgen excess (hirsutism, menstrual irregularity, hyperandrogenism), a phenotype resembling polycystic ovary syndrome (PCOS) (Draper et al. (2003) Nat. Genet. 34: 434-439).

The importance of the HPA axis in controlling glucocorticoid excursions is evident from the fact that disruption of homeostasis in the HPA axis by either excess or deficient secretion or action results in Cushing's syndrome or Addison's disease, respectively (Miller and Chrousos (2001) Endocrinology and Metabolism, eds. Felig and Frohman (McGraw-Hill, New York), 4^{th} Ed.: 387-524). Patients with Cushing's syndrome (a rare disease characterized by systemic glucocorticoid excess originating from the adrenal or pituitary tumors) or receiving glucocorticoid therapy develop reversible visceral fat obesity. Interestingly, the phenotype of Cushing's syndrome patients closely resembles that of Reaven's metabolic syndrome (also known as Syndrome X or insulin resistance syndrome) the symptoms of which include visceral obesity, glucose intolerance, insulin resistance, hypertension, type 2 diabetes and hyperlipidemia (Reaven (1993) Ann. Rev. Med. 44: 121-131). However, the role of glucocorticoids in prevalent forms of human obesity has remained obscure because circulating glucocorticoid concentrations are not elevated in the majority of metabolic syndrome patients. In fact, glucocorticoid action on target tissue depends not only on circulating levels but also on intracellular concentration, locally enhanced action of glucocorticoids in adipose tissue and skeletal muscle has been demonstrated in metabolic syndrome. Evidence has accumulated that enzyme activity of 11βHSD1, which regenerates active glucocorticoids from inactive forms and plays a central role in regulating intracellular glucocorticoid concentration, is commonly elevated in fat depots from obese individuals. This suggests a role for local glucocorticoid reactivation in obesity and metabolic syndrome.

Given the ability of 11βHSD1 to regenerate cortisol from inert circulating cortisone, considerable attention has been given to its role in the amplification of glucocorticoid function. 11βHSD1 is expressed in many key GR-rich tissues, including tissues of considerable metabolic importance such as liver, adipose, and skeletal muscle, and, as such, has been postulated to aid in the tissue-specific potentiation of glucocorticoid-mediated antagonism of insulin function. Considering a) the phenotypic similarity between glucocorticoid excess (Cushing's syndrome) and the metabolic syndrome with normal circulating glucocorticoids in the latter, as well as b) the ability of 11βHSD1 to generate active cortisol from inactive cortisone in a tissue-specific manner, it has been suggested that central obesity and the associated metabolic complications in syndrome X result from increased activity of 11βHSD1 within adipose tissue, resulting in ‘Cushing's disease of the omentum’ (Bujalska et al. (1997) Lancet 349: 1210-1213). Indeed, 11βHSD1 has been shown to be upregulated in adipose tissue of obese rodents and humans (Livingstone et al. (2000) Endocrinology 131: 560-563; Rask et al. (2001) J. Clin. Endocrinol. Metab. 86: 1418-1421; Lindsay et al. (2003) J. Clin. Endocrinol. Metab. 88: 2738-2744; Wake et al. (2003) J. Clin. Endocrinol. Metab. 88: 3983-3988).

Additional support for this notion has come from studies in mouse transgenic models. Adipose-specific overexpression of 11βHSD1 under the control of the aP2 promoter in mouse produces a phenotype remarkably reminiscent of human metabolic syndrome (Masuzaki et al. (2001) Science 294: 2166-2170; Masuzaki et al. (2003) J. Clinical Invest. 112: 83-90). Importantly, this phenotype occurs without an increase in total circulating corticosterone, but rather is driven by a local production of corticosterone within the adipose depots. The increased activity of 11βHSD1 in these mice (2-3 fold) is very similar to that observed in human obesity (Rask et al. (2001) J. Clin. Endocrinol. Metab. 86: 1418-1421). This suggests that local 11βHSD1-mediated conversion of inert glucocorticoid to active glucocorticoid can have profound influences whole body insulin sensitivity.

Based on this data, it would be predicted that the loss of 11βHSD1 would lead to an increase in insulin sensitivity and glucose tolerance due to a tissue-specific deficiency in active glucocorticoid levels. This is, in fact, the case as shown in studies with 11βHSD1-deficient mice produced by homologous recombination (Kotelevstev et al. (1997) Proc. Natl. Acad. Sci. 94: 14924-14929; Morton et al. (2001) J. Biol. Chem. 276: 41293-41300; Morton et al. (2004) Diabetes 53: 931-938). These mice are completely devoid of 11-keto reductase activity, confirming that 11βHSD1 encodes the only activity capable of generating active corticosterone from inert 11-dehydrocorticosterone. 11βHSD1-deficient mice are resistant to diet- and stress-induced hyperglycemia, exhibit attenuated induction of hepatic gluconeogenic enzymes (PEPCK, G6P), show increased insulin sensitivity within adipose, and have an improved lipid profile (decreased triglycerides and increased cardio-protective HDL). Additionally, these animals show resistance to high fat diet-induced obesity. Taken together, these transgenic mouse studies confirm a role for local reactivation of glucocorticoids in controlling hepatic and peripheral insulin sensitivity, and suggest that inhibition of 11βHSD1 activity may prove beneficial in treating a number of glucocorticoid-related disorders, including obesity, insulin resistance, hyperglycemia, and hyperlipidemia.

Data in support of this hypothesis has been published. Recently, it was reported that 11βHSD1 plays a role in the pathogenesis of central obesity and the appearance of the metabolic syndrome in humans. Increased expression of the 11βHSD1 gene is associated with metabolic abnormalities in obese women and that increased expression of this gene is suspected to contribute to the increased local conversion of cortisone to cortisol in adipose tissue of obese individuals (Engeli, et al., (2004) Obes. Res. 12: 9-17).

A new class of 11βHSD1 inhibitors, the arylsulfonamidothiazoles, was shown to improve hepatic insulin sensitivity and reduce blood glucose levels in hyperglycemic strains of mice (Barf et al. (2002) J. Med. Chem. 45: 3813-3815; Alberts et al. Endocrinology (2003) 144: 4755-4762). Furthermore, it was recently reported that selective inhibitors of 11βHSD1 can ameliorate severe hyperglycemia in genetically diabetic obese mice. Thus, 11βHSD1 is a promising pharmaceutical target for the treatment of the Metabolic Syndrome (Masuzaki, et al., (2003) Curr. Drug Targets Immune Endocr. Metabol. Disord. 3: 255-62).

A. Obesity and Metabolic Syndrome

As described above, multiple lines of evidence suggest that inhibition of 11βHSD1 activity can be effective in combating obesity and/or aspects of the metabolic syndrome cluster, including glucose intolerance, insulin resistance, hyperglycemia, hypertension, and/or hyperlipidemia. Glucocorticoids are known antagonists of insulin action, and reductions in local glucocorticoid levels by inhibition of intracellular cortisone to cortisol conversion should increase hepatic and/or peripheral insulin sensitivity and potentially reduce visceral adiposity. As described above, 11βHSD1 knockout mice are resistant to hyperglycemia, exhibit attenuated induction of key hepatic gluconeogenic enzymes, show markedly increased insulin sensitivity within adipose, and have an improved lipid profile. Additionally, these animals show resistance to high fat diet-induced obesity (Kotelevstev et al. (1997) Proc. Natl. Acad. Sci. 94: 14924-14929; Morton et al. (2001) J. Biol. Chem. 276: 41293-41300; Morton et al. (2004) Diabetes 53: 931-938). Thus, inhibition of 11βHSD1 is predicted to have multiple beneficial effects in the liver, adipose, and/or skeletal muscle, particularly related to alleviation of component(s) of the metabolic syndrome and/or obesity.

B. Pancreatic Function

Glucocorticoids are known to inhibit the glucose-stimulated secretion of insulin from pancreatic beta-cells (Billaudel and Sutter (1979) Horm. Metab. Res. 11: 555-560). In both Cushing's syndrome and diabetic Zucker fa/fa rats, glucose-stimulated insulin secretion is markedly reduced (Ogawa et al. (1992) J. Clin. Invest. 90: 497-504). 11βHSD1 mRNA and activity has been reported in the pancreatic islet cells of ob/ob mice and inhibition of this activity with carbenoxolone, an 11βHSD1 inhibitor, improves glucose-stimulated insulin release (Davani et al. (2000) J. Biol. Chem. 275: 34841-34844). Thus, inhibition of 11βHSD1 is predicted to have beneficial effects on the pancreas, including the enhancement of glucose-stimulated insulin release.

C. Cognition and Dementia

Mild cognitive impairment is a common feature of aging that may be ultimately related to the progression of dementia. In both aged animals and humans, inter-individual differences in general cognitive function have been linked to variability in the long-term exposure to glucocorticoids (Lupien et al. (1998) Nat. Neurosci. 1: 69-73). Further, dysregulation of the HPA axis resulting in chronic exposure to glucocorticoid excess in certain brain subregions has been proposed to contribute to the decline of cognitive function (McEwen and Sapolsky (1995) Curr. Opin. Neurobiol. 5: 205-216). 11βHSD1 is abundant in the brain, and is expressed in multiple subregions including the hippocampus, frontal cortex, and cerebellum (Sandeep et al. (2004) Proc. Natl. Acad. Sci. Early Edition: 1-6). Treatment of primary hippocampal cells with the 11βHSD1 inhibitor carbenoxolone protects the cells from glucocorticoid-mediated exacerbation of excitatory amino acid neurotoxicity (Rajan et al. (1996) J. Neurosci. 16: 65-70). Additionally, 11βHSD1-deficient mice are protected from glucocorticoid-associated hippocampal dysfunction that is associated with aging (Yau et al. (2001) Proc. Natl. Acad. Sci. 98: 4716-4721). In two randomized, double-blind, placebo-controlled crossover studies, administration of carbenoxolone improved verbal fluency and verbal memory (Sandeep et al. (2004) Proc. Natl. Acad. Sci. Early Edition: 1-6). Thus, inhibition of 11βHSD1 is predicted to reduce exposure to glucocorticoids in the brain and protect against deleterious glucocorticoid effects on neuronal function, including cognitive impairment, dementia, and/or depression.

D. Intra-Ocular Pressure

Glucocorticoids can be used topically and systemically for a wide range of conditions in clinical ophthalmology. One particular complication with these treatment regimens is corticosteroid-induced glaucoma. This pathology is characterized by a significant increase in intra-ocular pressure (IOP). In its most advanced and untreated form, IOP can lead to partial visual field loss and eventually blindness. IOP is produced by the relationship between aqueous humour production and drainage. Aqueous humour production occurs in the non-pigmented epithelial cells (NPE) and its drainage is through the cells of the trabecular meshwork. 11βHSD1 has been localized to NPE cells (Stokes et al. (2000) Invest. Ophthalmol. Vis. Sci. 41: 1629-1683; Rauz et al. (2001) Invest. Ophthalmol. Vis. Sci. 42: 2037-2042) and its function is likely relevant to the amplification of glucocorticoid activity within these cells. This notion has been confirmed by the observation that free cortisol concentration greatly exceeds that of cortisone in the aqueous humour (14:1 ratio). The functional significance of 11βHSD1 in the eye has been evaluated using the inhibitor carbenoxolone in healthy volunteers (Rauz et al. (2001) Invest. Ophthalmol. Vis. Sci. 42: 2037-2042). After seven days of carbenoxolone treatment, IOP was reduced by 18%. Thus, inhibition of 11βHSD1 in the eye is predicted to reduce local glucocorticoid concentrations and IOP, producing beneficial effects in the management of glaucoma and other visual disorders.

E. Hypertension

Adipocyte-derived hypertensive substances such as leptin and angiotensinogen have been proposed to be involved in the pathogenesis of obesity-related hypertension (Matsuzawa et al. (1999) Ann. N.Y. Acad. Sci. 892: 146-154; Wajchenberg (2000) Endocr. Rev. 21: 697-738). Leptin, which is secreted in excess in aP2-11βHSD1 transgenic mice (Masuzaki et al. (2003) J. Clinical Invest. 112: 83-90), can activate various sympathetic nervous system pathways, including those that regulate blood pressure (Matsuzawa et al. (1999) Ann. N.Y. Acad. Sci. 892: 146-154). Additionally, the renin-angiotensin system (RAS) has been shown to be a major determinant of blood pressure (Walker et al. (1979) Hypertension 1: 287-291). Angiotensinogen, which is produced in liver and adipose tissue, is the key substrate for renin and drives RAS activation. Plasma angiotensinogen levels are markedly elevated in aP2-11βHSD1 transgenic mice, as are angiotensin II and aldosterone (Masuzaki et al. (2003) J. Clinical Invest. 112: 83-90). These forces likely drive the elevated blood pressure observed in aP2-11βHSD1 transgenic mice. Treatment of these mice with low doses of an angiotensin II receptor antagonist abolishes this hypertension (Masuzaki et al. (2003) J. Clinical Invest. 112: 83-90). This data illustrates the importance of local glucocorticoid reactivation in adipose tissue and liver, and suggests that hypertension may be caused or exacerbated by 11βHSD1 activity. Thus, inhibition of 11βHSD1 and reduction in adipose and/or hepatic glucocorticoid levels is predicted to have beneficial effects on hypertension and hypertension-related cardiovascular disorders.

F. Bone Disease

Glucocorticoids can have adverse effects on skeletal tissues. Continued exposure to even moderate glucocorticoid doses can result in osteoporosis (Cannalis (1996) J. Clin. Endocrinol. Metab. 81: 3441-3447) and increased risk for fractures. Experiments in vitro confirm the deleterious effects of glucocorticoids on both bone-resorbing cells (also known as osteoclasts) and bone forming cells (osteoblasts). 11βHSD1 has been shown to be present in cultures of human primary osteoblasts as well as cells from adult bone, likely a mixture of osteoclasts and osteoblasts (Cooper et al. (2000) Bone 27: 375-381), and the 11βHSD1 inhibitor carbenoxolone has been shown to attenuate the negative effects of glucocorticoids on bone nodule formation (Bellows et al. (1998) Bone 23: 119-125). Thus, inhibition of 11βHSD1 is predicted to decrease the local glucocorticoid concentration within osteoblasts and osteoclasts, producing beneficial effects in various forms of bone disease, including osteoporosis.

Small molecule inhibitors of 11βHSD1 are currently being developed to treat or prevent 11βHSD1-related diseases such as those described above. For example, certain amide-based inhibitors are reported in WO 2004/089470, WO 2004/089896, WO 2004/056745, and WO 2004/065351.

Antagonists of 11βHSD1 have been evaluated in human clinical trials (Kurukulasuriya, et al., (2003) Curr. Med. Chem. 10: 123-53).

In light of the experimental data indicating a role for 11βHSD1 in glucocorticoid-related disorders, metabolic syndrome, hypertension, obesity, insulin resistance, hyperglycemia, hyperlipidemia, type 2 diabetes, androgen excess (hirsutism, menstrual irregularity, hyperandrogenism) and polycystic ovary syndrome (PCOS), therapeutic agents aimed at augmentation or suppression of these metabolic pathways, by modulating glucocorticoid signal transduction at the level of 11βHSD1 are desirable.

Furthermore, because the MR binds to aldosterone (its natural ligand) and cortisol with equal affinities, compounds that are designed to interact with the active site of 11βHSD1 (which binds to cortisone/cortisol) may also interact with the MR and act as antagonists. Because the MR is implicated in heart failure, hypertension, and related pathologies including atherosclerosis, arteriosclerosis, coronary artery disease, thrombosis, angina, peripheral vascular disease, vascular wall damage, and stroke, MR antagonists are desirable and may also be useful in treating complex cardiovascular, renal, and inflammatory pathologies including disorders of lipid metabolism including dyslipidemia or hyperlipoproteinaemia, diabetic dyslipidemia, mixed dyslipidemia, hypercholesterolemia, hypertriglyceridemia, as well as those associated with type 1 diabetes, type 2 diabetes, obesity, metabolic syndrome, and insulin resistance, and general aldosterone-related target-organ damage.

As evidenced herein, there is a continuing need for new and improved drugs that target 11βHSD1 and/or MR. The compounds, compositions and methods described herein help meet this and other needs.

### SUMMARY OF THE INVENTION

The present invention provides, inter alia, compounds of Formula Ia or Ib: 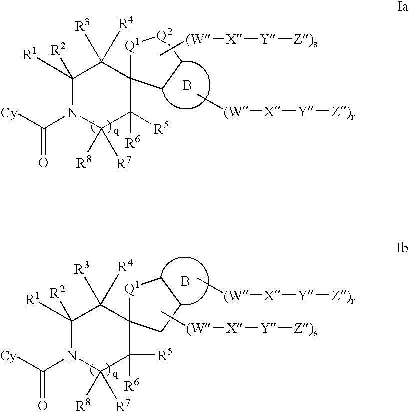
or pharmaceutically acceptable salts or prodrugs thereof, wherein constituent members are defined herein.

The present invention further provides compositions comprising compounds of the invention and a pharmaceutically acceptable carrier.

The present invention further provides methods of modulating 11βHSD1 or MR by contacting 11βHSD1 or MR with a compound of the invention. The present invention further provides methods of inhibiting 11βHSD1 or MR by contacting 11βHSD1 or MR with a compound of the invention.

The present invention further provides methods of inhibiting the conversion of cortisone to cortisol in a cell by contacting the cell with a compound of the invention.

The present invention further provides methods of inhibiting the production of cortisol in a cell by contacting the cell with a compound of the invention.

The present invention further provides methods of treating diseases associated with activity or expression of 11βHSD1 or MR.

### DETAILED DESCRIPTION

The present invention provides, inter alia, compounds of Formula Ia or Ib: 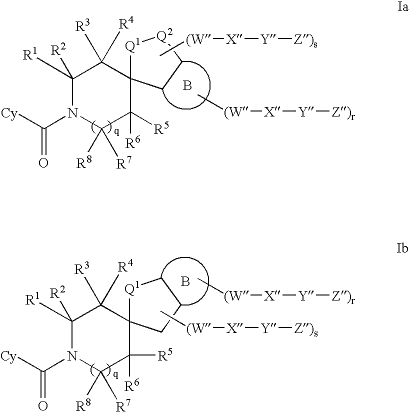
or pharmaceutically acceptable salt or prodrug thereof, wherein:

Cy is aryl, heteroaryl, cycloalkyl, or heterocycloalkyl, each optionally substituted by 1, 2, 3, 4 or 5 —U-T-W—X—Y-Z;

Q¹ is O, S, NH, CH₂, CO, CS, SO, SO₂, OCH₂, SCH₂, NHCH₂, CH₂CH₂, COCH₂, CONH, COO, SOCH₂, SONH, SO₂CH₂, or SO₂NH;

Q² is O, S, NH, CH₂, CO, CS, SO, SO₂, OCH₂, SCH₂, NHCH₂, CH₂CH₂, COCH₂, CONH, COO, SOCH₂, SONH, SO₂CH₂, or SO₂NH;

ring B is an aryl, heteroaryl, cycloalkyl, or heterocycloalkyl group fused with the ring containing Q¹ and Q²;

R¹, R², R³, R⁴, R⁵, R⁶, R⁷, and R⁸ are each, independently, H or —W′—X′—Y′-Z′;

or R¹ and R² together with the C atom to which they are attached form a 3-20 membered cycloalkyl group or a 3-20 membered heterocycloalkyl group optionally substituted by 1 or 2 —W″—X″—Y″-Z″;

or R³ and R⁴ together with the C atom to which they are attached form a 3-20 membered cycloalkyl group or a 3-20 membered heterocycloalkyl group optionally substituted by 1 or 2 —W″—X″—Y″-Z″;

or R⁵ and R⁶ together with the C atom to which they are attached form a 3-20 membered cycloalkyl group or a 3-20 membered heterocycloalkyl group optionally substituted by 1 or 2 —W″—X″—Y″-Z;

or R⁷ and R⁸ together with the C atom to which they are attached form a 3-20 membered cycloalkyl group or a 3-20 membered heterocycloalkyl group optionally substituted by 1 or 2 —W″—X″—Y″-Z″;

or R¹ and R⁵ together form an C₁₋₄ alkylene bridge optionally substituted by 1 or 2 —W″—X″—Y″-Z″;

or R³ and R⁵ together form an C₁₋₄ alkylene bridge optionally substituted by 1 or 2 U is absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;

T is absent, C₁₋₆ alkylenyl, C₂₋₆ alkenylenyl, C₂₋₆ alkynylenyl, aryl, aryloxy, cycloalkyl, heteroaryl, heteroaryloxy, or heterocycloalkyl, wherein said C₁₋₆ alkylenyl, C₂₋₆ alkenylenyl, C₂₋₆ alkynylenyl, cycloalkyl, heteroaryl or heterocycloalkyl is optionally substituted by one or more halo, CN, NO₂, OH, C₁₋₄ alkoxy, C₁₋₄ haloalkoxy, amino, C₁₋₄ alkylamino or C₂₋₈ dialkylamino;

W, W′ and W″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;

X, X′ and X″ are each, independently, absent, C₁₋₆ alkylenyl, C₂₋₆ alkenylenyl, C₂₋₆ alkynylenyl, aryl, cycloalkyl, heteroaryl or heterocycloalkyl, wherein said C₁₋₆ alkylenyl, C₂₋₆ alkenylenyl, C₂₋₆ alkynylenyl, cycloalkyl, heteroaryl or heterocycloalkyl is optionally substituted by one or more halo, CN, NO₂, OH, C₁₋₄ alkoxy, C₁₋₄ haloalkoxy, amino, C₁₋₄ alkylamino or C₂₋₈ dialkylamino;

Y, Y′ and Y″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;

Z, Z′ and Z″ are each, independently, H, halo, CN, NO₂, OH, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, aryl, cycloalkyl, heteroaryl or heterocycloalkyl, wherein each of said C₁₋₆ alkyl, C₂₋₆ alkenyl, C₂₋₆ alkynyl, aryl, cycloalkyl, heteroaryl or heterocycloalkyl is optionally substituted by 1, 2 or 3 halo, C₁₋₆ alkyl, C₁₋₆ hydroxyalkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, —C₁₋₄ alkyl-OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, NR^{c}S(O)₂R^{b} or S(O)₂NR^{c}R^{d};

wherein two —W—X—Y-Z together with the atom to which they are both attached optionally form a 3-20 membered cycloalkyl group or 3-20 membered heterocycloalkyl group optionally substituted by 1, 2 or 3 —W″—X″—Y″-Z″;

wherein two —W′—X′—Y′-Z′ together with the atom to which they are both attached optionally form a 3-20 membered cycloalkyl group or 3-20 membered heterocycloalkyl group optionally substituted by 1, 2 or 3 —W″—X″—Y″-Z″;

wherein —W—X—Y-Z is other than H;

wherein —W′—X′—Y′-Z′ is other than H;

wherein —W″—X″—Y″-Z″ is other than H;

R^{a} is H, C₁₋₆ alkyl, C₁₋₆ haloalkyl, C₂₋₆ alkenyl, C₂₋₆ alkynyl, aryl, cycloalkyl, heteroaryl or heterocycloalkyl;

R^{b} is H, C₁₋₆ alkyl, C₁₋₆ haloalkyl, C₂₋₆ alkenyl, C₂₋₆ alkynyl, aryl, cycloalkyl, heteroaryl or heterocycloalkyl;

R^{c} is H, C₁₋₆ alkyl, C₁₋₆ haloalkyl, C₂₋₆ alkenyl, C₂₋₆ alkynyl, aryl, cycloalkyl, arylalkyl, or cycloalkylalkyl;

or R^{c} and R^{d} together with the N atom to which they are attached form a 4-, 5-, 6- or 7-membered heterocycloalkyl group;

R^{e} and R^{f} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;

or R^{e} and R^{f} together with the N atom to which they are attached form a 4-, 5-, 6- or 7-membered heterocycloalkyl group;

q is 0, 1, or 2;

r is 0, 1 or 2; and

s is 0, 1 or 2.

In some embodiments, when the compound has Formula Ia, Q¹ is CO, and Q² is NH, then s is 0.

In some embodiments, when the compound has Formula Ia, Q¹ is CH₂, Q₂ is CH₂, and q is 1, then r is 1 or 2.

In some embodiments, when the compound has Formula Ib, Q¹ is NH, and Q² is CONH, then s is 0.

In some embodiments, when the compound has Formula Ib, Q¹ is CO, Q² is NH, then r is 1 or 2.

In some embodiments, Cy is other than cyclopropyl substituted by 1 or 2 —U-T-W—X—Y-Z.

In some embodiments, Z, Z′ and Z″ are each, independently, H, halo, CN, NO₂, OH, C₁₋₄ alkoxy, C₁₋₄ haloalkoxy, amino, C₁₋₄ alkylamino or C₂₋₈ dialkylamino, C₁₋₆ alkyl, C₂₋₆ alkenyl, C₂₋₆ alkynyl, aryl, cycloalkyl, heteroaryl or heterocycloalkyl, wherein said C₁₋₆ alkyl, C₂₋₆ alkenyl, C₂₋₆ alkynyl, aryl, cycloalkyl, heteroaryl or heterocycloalkyl is optionally substituted by 1, 2 or 3 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, or S(O)₂NR^{c}R^{d}.

In some embodiments, Cy is other than pyrrolidine, piperidine, or azepine.

In some embodiments, Cy is other than pyrrolidine, piperidine, or azepine substituted by 1, 2, or 3 —U-T-W—X—Y-Z.

In some embodiments, compounds of the invention have Formula Ia.

In some embodiments, compounds of the invention have Formula Ib.

In some embodiments, Cy is aryl or heteroaryl substituted by 1, 2, 3, 4 or 5 —U-T-W—X—Y-Z.

In some embodiments, Cy is aryl substituted by 1, 2, 3, 4 or 5 —U-T-W—X—Y-Z.

In some embodiments, Cy is phenyl substituted by 1, 2, 3, 4 or 5 —U-T-W—X—Y-Z.

In some embodiments, compounds of the invention have Formula Ia and Q¹ and Q² are each, independently, O, S, NH, CH₂, CO, CS, SO, or SO₂, wherein each of said NH and CH₂ is optionally substituted by —W″—X″—Y″-Z″.

In some embodiments, compounds of the invention have Formula Ia and Q¹ is O, NH, CO or CH₂ and Q² is CO, CH₂, NH, NHCH₂, or SO₂, wherein each of said NH, NHCH₂, and CH₂ is optionally substituted by —W″—X″—Y″-Z″.

In some embodiments, compounds of the invention have Formula Ia and Q¹ is O and Q² is CO.

In some embodiments, compounds of the invention have Formula Ib and Q¹ is O, NH, CO or CH₂ and Q² is CO, CH₂, NH, CH₂CH₂, NHCH₂, or SO₂, wherein each of said NH, CH₂CH₂, NHCH₂, and CH₂ is optionally substituted by —W″—X″—Y″-Z″.

In some embodiments, ring B is phenyl or pyridyl.

In some embodiments, R¹, R², R³, R⁴, R⁵, R⁶, R⁷, and R⁸ are each, independently, H or —W′—X′—Y′-Z′.

In some embodiments, R¹, R², R³, R⁴, R⁵, R⁶, R⁷, and R⁸ are each H.

In some embodiments, q is 0.

In some embodiments, q is 1.

In some embodiments, q is 2.

In some embodiments, s is 0.

In some embodiments, s is 1.

In some embodiments, s is 2.

In some embodiments, r is 0.

In some embodiments, r is 1.

In some embodiments, r is 2.

In some embodiments, —U-T-W—X—Y-Z is halo, cyano, C₁₋₄ cyanoalkyl, nitro, C₁₋₄ nitroalkyl, C₁₋₄ alkyl, C₁₋₄ haloalkyl, C₁₋₄ alkoxy, C₁₋₄ haloalkoxy, OH, C₁₋₈ alkoxyalkyl, amino, C₁₋₄ alkylamino, C₂₋₈ dialkylamino, aryl, heteroaryl, cycloalkyl, heterocycloalkyl, arylalkyl, heteroarylalkyl, cycloalkylalkyl, or heterocycloalkylalkyl.

In some embodiments, U and T are absent.

In some embodiments:
—U-T-W—X—Y-Z is halo, C₁₋₆ alkyl, amino, OH, OC(O)R^{b}, Z,
   —O-Z, —O—(C₁₋₄ alkyl)-Z, or —NHC(O)-Z; and
Z is aryl, cycloalkyl, heteroaryl or heterocycloalkyl, each optionally substituted by 1, 2 or 3 halo, C₁₋₆ alkyl, C₁₋₆ hydroxyalkyl, heterocycloalkyl, CN, OR^{a}, C(O)R^{b}, C(O)NR^{c}R^{d}, C(O)OR^{a}, —C₁₋₄ alkyl-OC(O)NR^{c}R^{d}, NR^{c}R^{d}, NR^{c}C(O)R^{d}, NR^{c}C(O)OR^{a}, S(O)₂R^{b}, or NR^{c}S(O)₂R^{b}.

In some embodiments:
—U-T-W—X—Y-Z is halo, C₁₋₆ alkyl, amino, OH, OC(O)R^{b}, Z,
   —O-Z, —O—(C₁₋₄ alkyl)-Z, or —NHC(O)-Z; and
Z is aryl, cycloalkyl, heteroaryl or heterocycloalkyl, each optionally substituted by 1, 2 or 3 halo, C₁₋₆ alkyl, C₁₋₆ hydroxyalkyl, 2-oxopyrrolidinyl, CN, OH, C₁₋₄ alkoxy, C(O)R^{b}, C(O)NR^{c}R^{d}, C(O)OR^{a}, —C₁₋₄ alkyl-OC(O)NR^{c}R^{d}, NR^{c}R^{d}, NR^{c}C(O)R^{d}, NR^{c}C(O)OR^{a}, S(O)₂R^{b}, or NR^{c}S(O)₂R^{b}.

In some embodiments:
—U-T-W—X—Y-Z is halo, C₁₋₆ alkyl, amino, OH, OC(O)R^{b}, Z,
   —O-Z, —O—(C₁₋₄ alkyl)-Z, or —NHC(O)-Z; and
Z is phenyl, naphthyl, cyclohexyl, pyridyl, pyrimidinyl, pyrazolyl, isoxazolyl, pyridazinyl, pyrazinyl, purinyl, quinoxalinyl, quinolinyl, 1,3-benzodioxolyl, piperidinyl, 1,2,3,6-tetrahydropyridinyl, morpholino, 2-oxo-pyrrolindinyl, 2-oxo-[1,3]oxazolidinyl, or piperizinyl, each optionally substituted by 1, 2 or 3 halo, C₁₋₆ alkyl, C₁₋₆ hydroxyalkyl, heterocycloalkyl, CN, OR^{a}, C(O)R^{b}, C(O)NR^{c}R^{d}, C(O)OR^{a}, —C₁₋₄ alkyl-OC(O)NR^{c}R^{d}, NR^{c}R^{d}, NR^{c}C(O)R^{d}, NR^{c}C(O)OR^{a}, S(O)₂R^{b}, or NR^{c}S(O)₂R^{b}.

In some embodiments, —W′—X′—Y′-Z′ is halo, cyano, C₁₋₄ cyanoalkyl, nitro, C₁₋₄ nitroalkyl, C₁₋₄ alkyl, C₁₋₄ haloalkyl, C₁₋₄ alkoxy, C₁₋₄ haloalkoxy, OH, C₁₋₈ alkoxyalkyl, amino, C₁₋₄ alkylamino, C₂₋₈ dialkylamino, aryl, heteroaryl, cycloalkyl, heterocycloalkyl, arylalkyl, heteroarylalkyl, cycloalkylalkyl, or heterocycloalkylalkyl.

In some embodiments, —W″—X″—Y″-Z″ is halo, cyano, C₁₋₄ cyanoalkyl, nitro, C₁₋₄ nitroalkyl, C₁₋₄ alkyl, C₁₋₄ haloalkyl, C₁₋₄ alkoxy, C₁₋₄ haloalkoxy, OH, C₁₋₈ alkoxyalkyl, amino, C₁₋₄ alkylamino, C₂₋₈ dialkylamino, aryl, heteroaryl, cycloalkyl, heterocycloalkyl, arylalkyl, heteroarylalkyl, cycloalkylalkyl, or heterocycloalkylalkyl.

In some embodiments, —W″—X″—Y″-Z″ is halo, cyano, C₁₋₄ cyanoalkyl, nitro, C₁₋₄ nitroalkyl, C₁₋₄ alkyl, C₁₋₄ haloalkyl, C₁₋₄ alkoxy, C₁₋₄ haloalkoxy, OH, C₁₋₈ alkoxyalkyl, amino, C₁₋₄ alkylamino, or C₂₋₈ dialkylamino.

In some embodiments, —W″—X″—Y″-Z″ is halo, cyano, or OH.

In some embodiments, the compounds of the invention have Formula II: 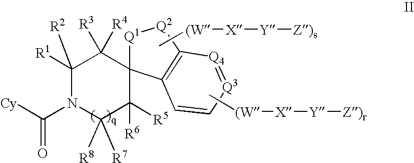
wherein:

Q³ and Q⁴ are each, independently, CH or N;

r is 0, 1 or 2; and

s is 0, 1 or 2.

In some embodiments, compounds of the invention have Formula II and Q¹ is O, NH, CH₂ or CO, wherein each of said NH and CH₂ is optionally substituted by —W″—X″—Y″-Z″.

In some embodiments, compounds of the invention have Formula II and Q² is O, S, NH, CH₂, CO, or SO₂, wherein each of said NH and CH₂ is optionally substituted by —W″—X″—Y″-Z″.

In some embodiments, compounds of the invention have Formula II and Q¹ and Q² is CO and the other is O, NH, or CH₂, wherein each of said NH and CH₂ is optionally substituted by —W″—X″—Y″-Z″.

In some embodiments, compounds of the invention have Formula II and one of Q¹ and Q² is CH₂ and the other is O, S, NH, or CH₂, wherein each of said NH and CH₂ is optionally substituted by In some embodiments, compounds of the invention have Formula II and one of Q¹ and Q² is O and the other is CO or CONH, wherein said CONH is optionally substituted by —W″—X″—Y″-Z″.

In some embodiments, compounds of the invention have Formula II and Q³ is CH optionally substituted by —W″—X″—Y″-Z″.

In some embodiments, compounds of the invention have Formula II and Q³ is N.

In some embodiments, compounds of the invention have Formula II and Q⁴ is CH optionally substituted by —W″—X″—Y″-Z″.

In some embodiments, compounds of the invention have Formula II and Q⁴ is N.

In some embodiments, compounds of the invention have Formula II and r is 0 or 1.

In some embodiments, compounds of the invention have Formula II and s is 0 or 1.

In some embodiments, compounds of the invention have Formula III: 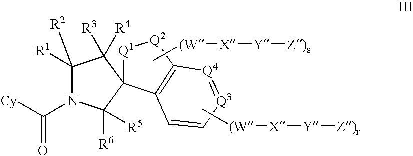
wherein:

Q³ and Q⁴ are each, independently, CH or N;

r is 0, 1 or 2; and

s is 0, 1 or 2.

In some embodiments, compounds of the invention have Formula III and Q¹ is O, NH, CH₂ or CO, wherein each of said NH and CH₂ is optionally substituted by —W″—X″—Y″-Z″.

In some embodiments, compounds of the invention have Formula III and Q² is O, S, NH, CH₂, CO, or SO₂, wherein each of said NH and CH₂ is optionally substituted by —W″—X″—Y″-Z″.

In some embodiments, compounds of the invention have Formula III and one of Q¹ and Q² is CO and the other is O, NH, or CH₂, wherein each of said NH and CH₂ is optionally substituted by —W″—X″—Y″-Z″.

In some embodiments, compounds of the invention have Formula III and one of Q¹ and Q² is CH₂ and the other is O, S, NH, or CH₂, wherein each of said NH and CH₂ is optionally substituted by In some embodiments, compounds of the invention have Formula III and one of Q¹ and Q² is O and the other is CO or CONH, wherein said CONH is optionally substituted by —W″—X″—Y″-Z″.

In some embodiments, compounds of the invention have Formula III and Q³ is CH optionally substituted by —W″—X″—Y″-Z″.

In some embodiments, compounds of the invention have Formula III and Q³ is N.

In some embodiments, compounds of the invention have Formula III and Q⁴ is CH optionally substituted by —W″—X″—Y″-Z″.

In some embodiments, compounds of the invention have Formula III and Q⁴ is N.

In some embodiments, compounds of the invention have Formula III and r is 0 or 1.

In some embodiments, compounds of the invention have Formula III and s is 0 or 1.

In some embodiments, Q¹ and Q² are selected to form a 1-, 2-, or 3-atom spacer. In further embodiments, Q¹ and Q² when bonded together form a spacer group having other than an O—O or O—S ring-forming bond.

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “n-membered” where n is an integer typically describes the number of ring-forming atoms in a moiety where the number of ring-forming atoms is n. For example, piperidinyl is an example of a 6-membered heterocycloalkyl ring and 1,2,3,4-tetrahydro-naphthalene is an example of a 10-membered cycloalkyl group.

For compounds of the invention in which a variable appears more than once, each variable can be a different moiety selected from the Markush group defining the variable. For example, where a structure is described having two R groups that are simultaneously present on the same compound; the two R groups can represent different moieties selected from the Markush group defined for R. In another example, when an optionally multiple substituent is designated in the form: 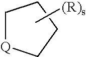
then it is understood that substituent R can occur s number of times on the ring, and R can be a different moiety at each occurrence. Further, in the above example, should the variable Q be defined to include hydrogens, such as when Q is said to be CH₂, NH, etc., any floating substituent such as R in the above example, can replace a hydrogen of the Q variable as well as a hydrogen in any other non-variable component of the ring.

It is further intended that the compounds of the invention are stable. As used herein “stable” refers to a compound that is sufficiently robust to survive isolation to a useful degree of purity from a reaction mixture, and preferably capable of formulation into an efficacious therapeutic agent.

As used herein, the term “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 20, from 2 to about 20, from 1 to about 10, from 1 to about 8, from 1 to about 6, from 1 to about 4, or from 1 to about 3 carbon atoms. The term “alkylenyl” refers to a divalent alkyl linking group.

As used herein, “alkenyl” refers to an alkyl group having one or more double carbon-carbon bonds. Example alkenyl groups include ethenyl, propenyl, cyclohexenyl, and the like. The term “alkenylenyl” refers to a divalent linking alkenyl group.

As used herein, “alkynyl” refers to an alkyl group having one or more triple carbon-carbon bonds. Example alkynyl groups include ethynyl, propynyl, and the like. The term “alkynylenyl” refers to a divalent linking alkynyl group.

As used herein, “haloalkyl” refers to an alkyl group having one or more halogen substituents. Example haloalkyl groups include CF₃, C₂F₅, CHF₂, CCl₃, CHCl₂, C₂Cl₅, and the like.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, indenyl, and the like. In some embodiments, aryl groups have from 6 to about 20 carbon atoms.

As used herein, “cycloalkyl” refers to non-aromatic cyclic hydrocarbons including cyclized alkyl, alkenyl, and alkynyl groups. Cycloalkyl groups can include mono- or polycyclic (e.g., having 2, 3 or 4 fused rings) ring systems as well as spiro ring systems. Ring-forming carbon atoms of a cycloalkyl group can be optionally substituted by oxo or sulfido. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (i.e., having a bond in common with) to the cycloalkyl ring, for example, benzo or thienyl derivatives of pentane, pentene, hexane, and the like.

As used herein, “heteroaryl” groups refer to an aromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

As used herein, “heterocycloalkyl” refers to non-aromatic heterocycles where one or more of the ring-forming atoms is a heteroatom such as an O, N, or S atom. Example “heterocycloalkyl” groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Ring-forming carbon atoms and heteroatoms of a heterocycloalkyl group can be optionally substituted by oxo or sulfido. Also included in the definition of heterocycloalkyl are moieties that have one or more aromatic rings fused (i.e., having a bond in common with) to the nonaromatic heterocyclic ring, for example phthalimidyl, naphthalimidyl, and benzo derivatives of heterocycles such as indolene and isoindolene groups. In some embodiments, the heterocycloalkyl group has from 1 to about 20 carbon atoms, and in further embodiments from about 3 to about 20 carbon atoms. In some embodiments, the heterocycloalkyl group contains 3 to about 14, 3 to about 7, or 5 to 6 ring-forming atoms. In some embodiments, the heterocycloalkyl group has 1 to about 4, 1 to about 3, or 1 to 2 heteroatoms. In some embodiments, the heterocycloalkyl group contains 0 to 3 double bonds. In some embodiments, the heterocycloalkyl group contains 0 to 2 triple bonds.

As used herein, “halo” or “halogen” includes fluoro, chloro, bromo, and iodo.

As used herein, “alkoxy” refers to an —O-alkyl group. Example alkoxy groups include methoxy, ethoxy, propoxy (e.g., n-propoxy and isopropoxy), t-butoxy, and the like.

As used here, “haloalkoxy” refers to an —O-haloalkyl group. An example haloalkoxy group is OCF₃.

As used herein, “arylalkyl” refers to alkyl substituted by aryl and “cycloalkylalkyl” refers to alkyl substituted by cycloalkyl. An example arylalkyl group is benzyl.

As used herein, “amino” refers to NH₂.

As used herein, “alkylamino” refers to an amino group substituted by an alkyl group.

As used herein, “dialkylamino” refers to an amino group substituted by two alkyl groups.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. An example method includes fractional recrystallizaion using a “chiral resolving acid” which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds of the invention also include tautomeric forms, such as keto-enol tautomers.

Compounds of the invention can also include all isotopes of atoms occurring in the intermediates or final compounds. Isotopes include those atoms having the same atomic number but different mass numbers. For example, isotopes of hydrogen include tritium and deuterium.

Compounds of the invention further include hydrates and solvates.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts or the quaternary ammonium salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

The present invention also includes prodrugs of the compounds described herein. As used herein, “prodrugs” refer to any covalently bonded carriers which release the active parent drug when administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Examples of prodrugs include, but are not limited to, acetate, formate and benzoate derivatives of alcohol and amine functional groups in the compounds of the invention. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

Synthesis

The novel compounds of the present invention can be prepared in a variety of ways known to one skilled in the art of organic synthesis. The compounds of the present invention can be synthesized using the methods as hereinafter described below, together with synthetic methods known in the art of synthetic organic chemistry or variations thereon as appreciated by those skilled in the art.

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

The processes described herein can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatograpy (HPLC) or thin layer chromatography.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

The compounds of the invention can be prepared, for example, using the reaction pathways and techniques as described below.

A series of carboxamides of formulas 1-3 and 1-5 can be prepared by the method outlined in Scheme 1. Carboxylic acids 1-1 can be coupled to amine 1-2 or 1-4 using a coupling reagent such as BOP to provide the carboxamides products.

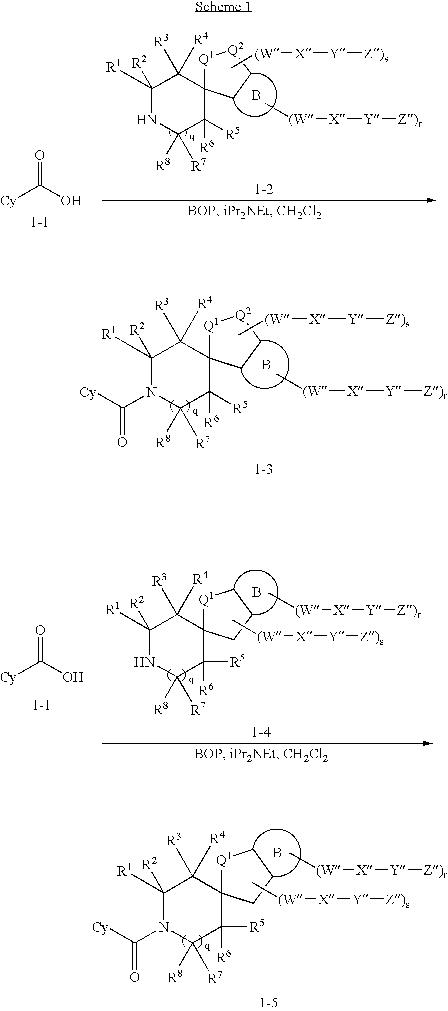

Scheme 2 shows further elaboration of hydroxyl substituted phenyl. Phenols of formula 2-1 can be coupled with boronic acid RB(OH)₂ (R is aryl, heteroaryl, cycloalkyl, heterocycloalkyl, alkyl, arylalkyl, heteroarylalkyl, cycloalkylalkyl, heterocycloalkylalkyl, etc.) catalysized by cupric acetate and TEPMO or coupled with RX (X=a leaving group such as halo) in potassium carbonate and a suitable solvent such as DMF or DMSO to form ethers of formula 2-2.

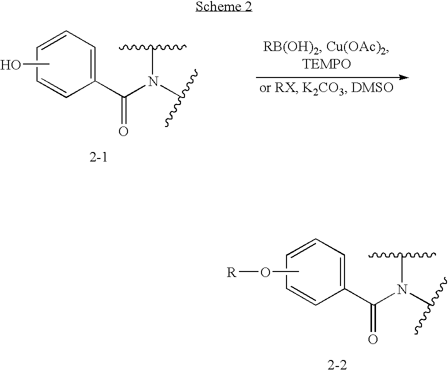

A series of carboxylic acids of formula 3-4 can be prepared by the method outlined in Scheme 3. Pd catalyzed coupling of compound 3-1 with any of a variety of substituted aryl or heteroaryl bromides (3-2) can afford the product 3-3. Hydrolysis of the methyl ester yields the carboxylic acid 3-4. These carboxylic acids can be coupled to amines as described in Scheme 1.

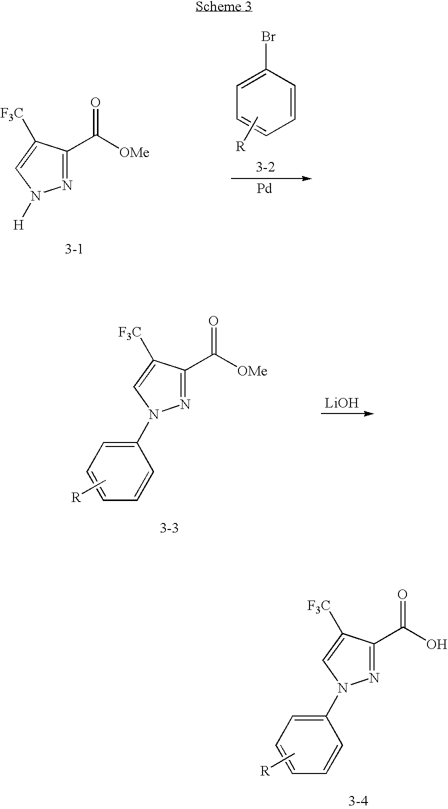

Pyrrolidines 4-4 can also be prepared according to Scheme 4. Halogen metal exchange between aryl iodide 4-1 and isopropylmagnesium bromide followed by reaction with N-Boc-3-oxo-pyrrolidine provides protected spiral lactone 4-3 which upon acidic cleavage of the Boc group yields the desired pyrrolidine 4-4.

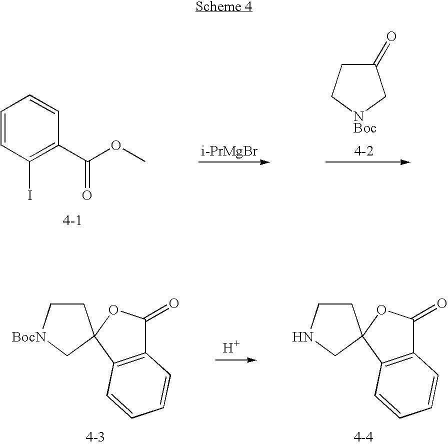

Alternatively, pyrrolidines 5-4 can be prepared according to Scheme 5. Ortho lithiation of carboxylic acid 5-1, followed by reaction of the resulting organolithium with N-Boc-3-oxo-pyrrolidine (5-2) yields protected spiral lactone 5-3, which upon acidic cleavage of the Boc group provides the desired pyrrolidine 5-4.

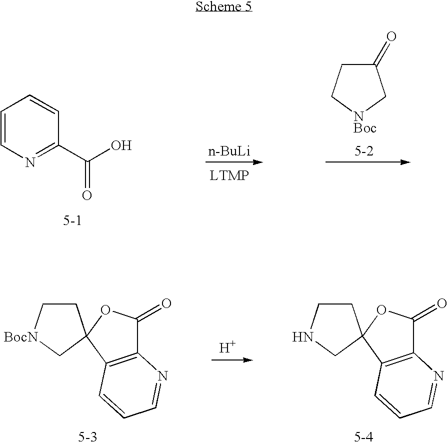

Pyrrolidines 6-5 can be prepared according to the method outlined in Scheme 6. 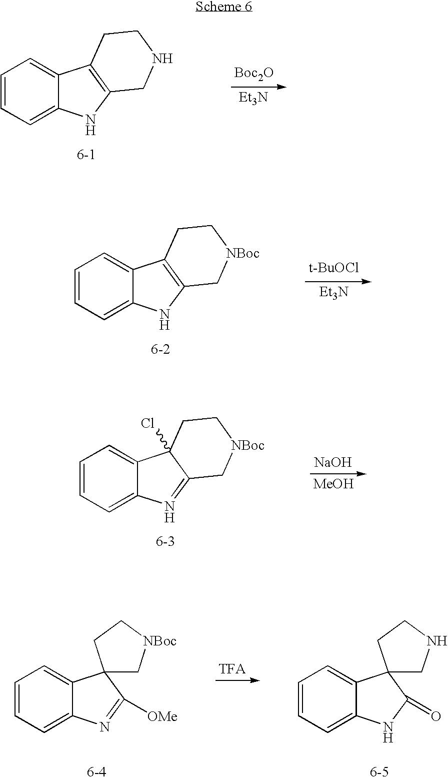
Methods

Compounds of the invention can modulate activity of 11βHSD1 and/or MR. The term “modulate” is meant to refer to an ability to increase or decrease activity of an enzyme or receptor. Accordingly, compounds of the invention can be used in methods of modulating 11βHSD1 and/or MR by contacting the enzyme or receptor with any one or more of the compounds or compositions described herein. In some embodiments, compounds of the present invention can act as inhibitors of 11βHSD1 and/or MR. In further embodiments, the compounds of the invention can be used to modulate activity of 11βHSD1 and/or MR in an individual in need of modulation of the enzyme or receptor by administering a modulating amount of a compound of the invention.

The present invention further provides methods of inhibiting the conversion of cortisone to cortisol in a cell, or inhibiting the production of cortisol in a cell, where conversion to or production of cortisol is mediated, at least in part, by 11βHSD1 activity. Methods of measuring conversion rates of cortisone to cortisol and vice versa, as well as methods for measuring levels of cortisone and cortisol in cells, are routine in the art.

The present invention further provides methods of increasing insulin sensitivity of a cell by contacting the cell with a compound of the invention. Methods of measuring insulin sensitivity are routine in the art.

The present invention further provides methods of treating disease associated with activity or expression, including abnormal activity and overexpression, of 11βHSD1 and/or MR in an individual (e.g., patient) by administering to the individual in need of such treatment a therapeutically effective amount or dose of a compound of the present invention or a pharmaceutical composition thereof. Example diseases can include any disease, disorder or condition that is directly or indirectly linked to expression or activity of the enzyme or receptor. An 11βHSD1-associated disease can also include any disease, disorder or condition that can be prevented, ameliorated, or cured by modulating enzyme activity.

Examples of 11βHSD1-associated diseases include obesity, diabetes, glucose intolerance, insulin resistance, hyperglycemia, hypertension, hyperlipidemia, cognitive impairment, dementia, depression (e.g., psychotic depression), glaucoma, cardiovascular disorders, osteoporosis, and inflammation. Further examples of 11βHSD1-associated diseases include metabolic syndrome, type 2 diabetes, androgen excess (hirsutism, menstrual irregularity, hyperandrogenism) and polycystic ovary syndrome (PCOS).

The present invention further provides methods of modulating MR activity by contacting the MR with a compound of the invention, pharmaceutically acceptable salt, prodrug, or composition thereof. In some embodiments, the modulation can be inhibition. In further embodiments, methods of inhibiting aldosterone binding to the MR (optionally in a cell) are provided. Methods of measuring MR activity and inhibition of aldosterone binding are routine in the art.

The present invention further provides methods of treating a disease associated with activity or expression of the MR. Examples of diseases associated with activity or expression of the MR include, but are not limited to hypertension, as well as cardiovascular, renal, and inflammatory pathologies such as heart failure, atherosclerosis, arteriosclerosis, coronary artery disease, thrombosis, angina, peripheral vascular disease, vascular wall damage, stroke, dyslipidemia, hyperlipoproteinaemia, diabetic dyslipidemia, mixed dyslipidemia, hypercholesterolemia, hypertriglyceridemia, and those associated with type 1 diabetes, type 2 diabetes, obesity metabolic syndrome, insulin resistance and general aldosterone-related target organ damage.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal. In some embodiments, the cell is an adipocyte, a pancreatic cell, a hepatocyte, neuron, or cell comprising the eye.

As used herein, the term “contacting” refers to the bringing together of indicated moieties in an in vitro system or an in vivo system. For example, “contacting” the 11βHSD1 enzyme with a compound of the invention includes the administration of a compound of the present invention to an individual or patient, such as a human, having 11βHSD1, as well as, for example, introducing a compound of the invention into a sample containing a cellular or purified preparation containing the 11βHSD1 enzyme.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician, which includes one or more of the following:

(1) preventing the disease; for example, preventing a disease, condition or disorder in an individual who may be predisposed to the disease, condition or disorder but does not yet experience or display the pathology or symptomatology of the disease (non-limiting examples are preventing metabolic syndrome, hypertension, obesity, insulin resistance, hyperglycemia, hyperlipidemia, type 2 diabetes, androgen excess (hirsutism, menstrual irregularity, hyperandrogenism) and polycystic ovary syndrome (PCOS);

(2) inhibiting the disease; for example, inhibiting a disease, condition or disorder in an individual who is experiencing or displaying the pathology or symptomatology of the disease, condition or disorder (i.e., arresting further development of the pathology and/or symptomatology) such as inhibiting the development of metabolic syndrome, hypertension, obesity, insulin resistance, hyperglycemia, hyperlipidemia, type 2 diabetes, androgen excess (hirsutism, menstrual irregularity, hyperandrogenism) or polycystic ovary syndrome (PCOS), stabilizing viral load in the case of a viral infection; and

(3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of metabolic syndrome, hypertension, obesity, insulin resistance, hyperglycemia, hyperlipidemia, type 2 diabetes, androgen excess (hirsutism, menstrual irregularity, hyperandrogenism) and polycystic ovary syndrome (PCOS), or lowering viral load in the case of a viral infection.

Pharmaceutical Formulations and Dosage Forms

When employed as pharmaceuticals, the compounds of the invention can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, one or more of the compounds of the invention above in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions can be formulated in a unit dosage form, each dosage containing from about 5 to about 100 mg, more usually about 10 to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral adminstration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compounds of the invention can also be formulated in combination with one or more additional active ingredients which can include any pharmaceutical agent such as anti-viral agents, antibodies, immune suppressants, anti-inflammatory agents and the like.

Labeled Compounds and Assay Methods

Another aspect of the present invention relates to labeled compounds of the invention (radio-labeled, fluorescent-labeled, etc.) that would be useful not only in radio-imaging but also in assays, both in vitro and in vivo, for localizing and quantitating the enzyme in tissue samples, including human, and for identifying ligands by inhibition binding of a labeled compound. Accordingly, the present invention includes enzyme assays that contain such labeled compounds.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁷O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro receptor labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled compound” is a compound that has incorporated at least one radionuclide. In some embodiments the radionuclide is selected from the group consisting of ³H, ¹⁴C, ¹²⁵I, ³⁵S and ⁸²Br.

In some embodiments, the labeled compounds of the present invention contain a fluorescent lable.

Synthetic methods for incorporating radio-isotopes and fluorescent labels into organic compounds are are well known in the art.

A labeled compound of the invention (radio-labeled, fluorescent-labeled, etc.) can be used in a screening assay to identify/evaluate compounds. For example, a newly synthesized or identified compound (i.e., test compound) which is labeled can be evaluated for its ability to bind a 11βHSD1 or MR by monitering its concentration variation when contacting with the 11βHSD1 or MR, through tracking the labeling. For another example, a test compound (labeled) can be evaluated for its ability to reduce binding of another compound which is known to bind to 11βHSD1 or MR (i.e., standard compound). Accordingly, the ability of a test compound to compete with the standard compound for binding to the 11βHSD1 or MR directly correlates to its binding affinity. Conversely, in some other screening assays, the standard compound is labled and test compounds are unlabeled. Accordingly, the concentration of the labled standard compound is monitored in order to evaluate the competition between the standard compound and the test compound, and the relative binding affinity of the test compound is thus ascertained.

Kits

The present invention also includes pharmaceutical kits useful, for example, in the treatment or prevention of 11βHSD1- or MR-associated diseases or disorders, obesity, diabetes and other diseases referred to herein which include one or more containers containing a pharmaceutical composition comprising a therapeutically effective amount of a compound of the invention. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results. The compound of the Examples were found to inhibitors of 11βHSD1 and/or MR according to one or more of the assays provided herein.

### EXAMPLES

### Example 1

### (1R)-1′-(4-Phenoxybenzoyl)-3H-spiro[2-benzofuran-1,3′-pyrrolidin]-3-one

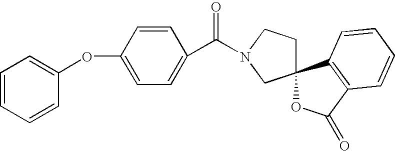

### Step1. Benzyl 3-oxo-1′H,3H-spiro[2-benzofuran-1,3′-pyrrolidine-1′carboxylate

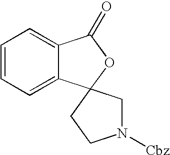

To a solution of methyl-2-iodobenzoate (8.8 mL, 0.060 mol) in THF (300 mL) at −60° C. was slowly added a solution of isopropylmagnesium bromide in THF (1.0 M, 66.0 mL), and the mixture was stirred below −50-C for 1 h. A solution of benzyl-3-oxopyrrolidine-1-carboxylate (11.0 g, 0.05 mol) in THF (20.0 mL) was added to the above mixture and the reaction mixture was stirred below −20° C. for 2 h. The reaction was quenched by the addition of saturated NH₄Cl aqueous solution and the resulting mixture was extracted with ethyl acetate several times. The combined extract was washed with water followed by brine, dried (NaSO₄), and concentrated in-vacuo. The product was purified by CombiFlash eluting with hexane/ethyl acetate.

### Step 2. [(1S)-7,7-dimethyl-2-oxobicyclo[2.2.1]hept-1-yl]methanesulfonic acid-(1R)-3H-spiro[2-benzofuran-1,3′-pyrrolidin]-3-one (1:1)

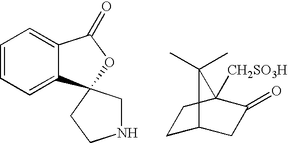

Palladium on carbon (10%, 0.5 g) was added to a solution of benzyl 3-oxo-1′H,3H-spiro[2-benzofuran-1,3′-pyrrolidine]-1′carboxylate (5.0 g, 15.5 mmol) in methanol (100 mL) and the mixture was stirred under a hydrogen balloon for 4 h (HPLC completion). The volatiles were removed under vacuum and the residue was dissolved in acetonitrile (200 mL) and (1S)-(+)-10-camphorsulfonic acid (3.6 g, 15.5 mmol) in acetonitrile (20 mL) was then slowly added at 50° C. After stirring for 1 h, the precipitate was filtered, washed with cold acetonitrile, and dried to afford the desired enantiomer (CSA salt) as a white solid (4.73 g, 41%). LC-MS: 190.1 (M+H)⁺.

### Step 3

N,N-Diisopropylethylamine (50 μL, 0.3 mmol) was added to a mixture of 4-phenoxybenzoic acid (22.5 mg, 0.1 mmol), (1S)-(+)-10-camphorsulfonic acid-3H-spiro-[2-benzofuran-1,3′-pyrrolidin]-3-one (1:1) 42.1 mg, 0.01 mmol) and benzotriazol-1-yloxytris(dimethylamino)phosphonium hexafluorophosphate (BOP) (57.0 mg, 0.13 mmol) in DMF (0.5 mL) at room temperature and the reaction was stirred for 5 h (HPLC completion). The product was purified by prep-HPLC. LC-MS: 386.1 (M+H)⁺.

### Example 2

### 1′-(3-Phenoxybenzoyl)-3H-spiro[2-benzofuran-1,3′-pyrrolidin]-3-one

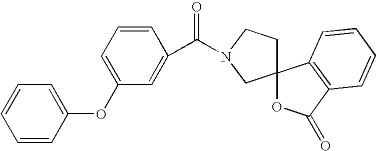

N,N-Diisopropylethylamine (50 μL, 0.3 mmol) was added to the mixture of 3-phenoxybenzoic acid (22.5 mg, 0.1 mmol), (1S)-(+)-10-camphorsulfonic acid-3H-spiro-[2-benzofuran-1,3′-pyrrolidin]-3-one (1:1) 42.1 mg, 0.01 mmol), and BOP (57.0 mg, 0.13 mmol) in DMF (0.5 mL) at room temperature and the reaction was stirred for 5 h (HPLC completion). The product was purified by prep-HPLC. LC-MS: 386.1 (M+H)⁺.

### Example 3

### (1R)-1′-(3-Bromobenzoyl)-3H-spiro[2-benzofuran-1,3′-pyrrolidin]-3-one

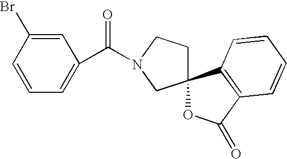

This compound was prepared using procedures analogous to example 1. LC-MS: 370.0/372.0 (M+H)⁺.

### Example 4

### (1R)-1′-(4-(Benzyloxy)benzoyl]-3H-spiro[2-benzofuran-1,3′-pyrrolidin]-3-one

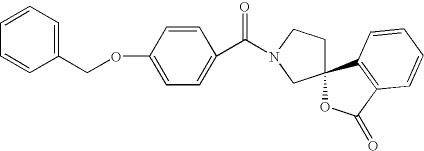

### Step1. (1R)-1′-(4-hydroxybenzoyl)-3H-spiro[2-benzofuran-1,3′-pyrrolidin]-3-one

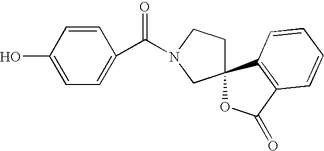

This compound was prepared using procedures analogous to example 1. LC-MS: 310.1 (M+H)⁺.

### Step 2

A mixture of (1R)-1′-(4-hydroxybenzoyl)-3H-spiro[2-benzofuran-1,3′-pyrrolidin]-3-one (10.0 mg, 0.03 mmol), benzylbromide (8 μL, 0.06 mmol), potassium carbonate (14.0 mg, 0.1 mmol) in DMSO (0.5 mL) was stirred at 120° C. for 2 h (HPLC completion). The product was purified by prep-HPLC. LC-MS: 400.1 (M+H)⁺.

### Example 5

### (1R)-1′-[4-(Cyclohexyloxy)benzoyl]-3H-spiro[2-benzofuran-1,3′-pyrrolidin]-3-one

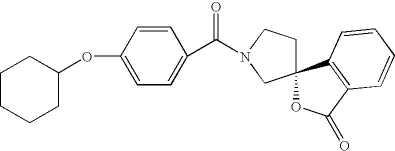

This compound was prepared using procedures analogous to example 4. LC-MS: 392.2 (M+H)⁺.

### Example 6

### (1R)-1′-[4-(Pyridin-2-yloxy)benzoyl]-3H-spiro[2-benzofuran-1,3′-pyrrolidin]-3-one

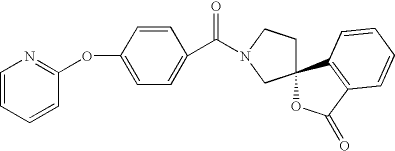

This compound was prepared using procedures analogous to example 4. LC-MS: 387.1 (M+H)⁺.

### Example 7

### (1R)-1′-[4-(Pyrazin-2-yloxy)benzoyl]-3H-spiro[2-benzofuran-1,3′-pyrrolidin]-3-one

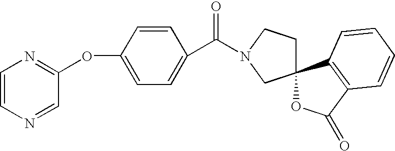

This compound was prepared using procedures analogous to example 4. LC-MS: 388.1 (M+H)⁺.

### Example 8

### (1R)-1′-[3-(2-Chlorophenoxy)benzoyl]-3H-spiro[2-benzofuran-1,3′-pyrrolidin]-3-one

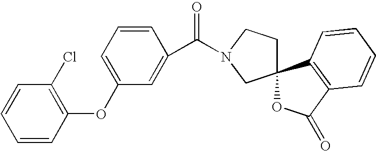

### Step1. (1R)-1′-(4-hydroxybenzoyl)-3H-spiro[2-benzofuran-1,3′-pyrrolidin]-3-one

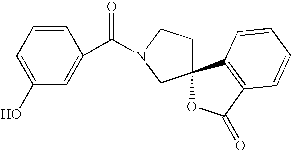

This compound was prepared using procedures analogous to example 1. LC-MS: 310.1 (M+H)⁺.

### Step 2. (1R)-1′-[3-(2-Chlorophenoxy)benzoyl]-3H-spiro[2-benzofuran-1,3′-pyrrolidin]-3-one

A mixture of 4 Å molecular sieves (40 mg), 2-chlorophenylboronic acid (15.0 mg, 0.10 mmol), cupric acetate (2.0 mg, 0.01 mmol), TEMPO (8.6 mg, 0.055 mmol), pyridine (8.0 μL, 0.1 mmol), (1R)-1′-(3-hydroxybenzoyl)-3H-spiro[2-benzofuran-1,3′-pyrrolidin]-3-one (15.5 mg, 0.050 mmol) in methylene chloride (3.0 mL) was stirred at 50° C. under an atmosphere of oxygen for 3 days. The reaction was cooled to room temperature and filtered through a pad of Celite. The filtrate was concentrated in-vacuo and the product was purified by prep-HPLC. LC-MS: 420.0/422.0 (M+H)⁺.

### Example 9

### (1R)-1′-[3-(3-Chlorophenoxy)benzoyl]-3H-spiro[2-benzofuran-1,3′-pyrrolidin]-3-one

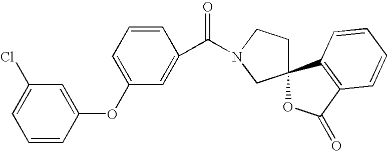

This compound was prepared using procedures analogous to example 8. LC-MS: 420.0/422.0 (M+H)⁺.

### Example 10

### (1R)-1′-[3-(4-Chlorophenoxy)benzoyl]-3H-spiro[2-benzofuran-1,3′-pyrrolidin]-3-one

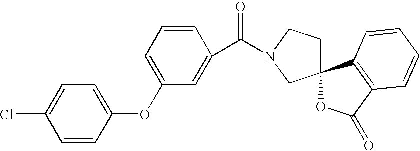

This compound was prepared using procedures analogous to example 8. LC-MS: 420.0/422.0 (M+H)⁺.

### Example 11

### (1R)-1′-(Biphenyl-4-ylcarbonyl)-3H-spiro[2-benzofuran-1,3′-pyrrolidin]-3-one

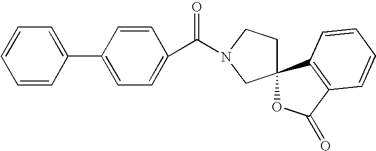

N,N-Diisopropylethylamine (26.0 μL, 0.15 mmol) was added to a solution of biphenyl-4-carbonyl chloride (11.3 mg, 0.05 mmol) and (1S)-(+)-10-camphorsulfonic acid-3H-spiro-[2-benzofuran-1,3′-pyrrolidin]-3-one (1:1) {21.0 mg, 0.05 mmol, also known as [(1S)-7,7-dimethyl-2-oxobicyclo[2.2.1]hept-1-yl]methanesulfonic acid-3H-spiro[2-benzofuran-1,3′-pyrrolidin]-3-one} in CH₂Cl₂ (0.5 mL) at 0° C. and the mixture was stirred overnight and the product was purified by prep-HPLC. LC-MS: 370.1. (M+H)⁺.

### Example 12

### (1R)-1′-[2-Fluoro-4-(pyrazin-2-yloxy)benzoyl]-3H-spiro[2-benzofuran-1,3′-pyrrolidin]-3-one

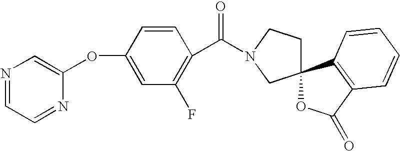

This compound was prepared using procedures analogous to example 4. LC-MS: 406.1 (M+H)⁺.

### Example 13

### (1R)-1′-[2-Chloro-4-(pyrazin-2-yloxy)benzoyl]-3H-spiro[2-benzofuran-1,3′-pyrrolidin]-3-one

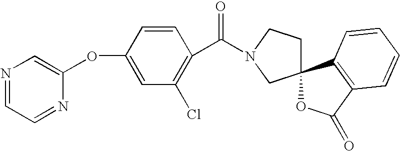

This compound was prepared using procedures analogous to example 4. LC-MS: 422.0 (M+H)⁺.

### Example 14

### (1R)-1′-{2-Chloro-4-[(3-chloropyrazin-2-yl)oxy]benzoyl}-3H-spiro[2-benzofuran-1,3′-pyrrolidin]-3-one

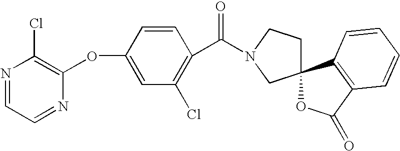

This compound was prepared using procedures analogous to example 4. LC-MS: 456.0/458.0 (M+H)⁺.

### Example 15

### (1R)-1′-{2-Chloro-4-[(3,6-dimethylpyrazin-2-yl)oxy]benzoyl}-3H-spiro[2-benzofuran-1,3′-pyrrolidin]-3-one

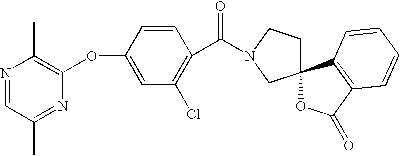

This compound was prepared using procedures analogous to example 4. LC-MS: 450.1/452.1 (M+H)⁺.

### Example 16

### (1R)-1′-[2-Chloro-4-(quinoxalin-2-yloxy)benzoyl]-3H-spiro[2-benzofuran-1,3′-pyrrolidin]-3-one

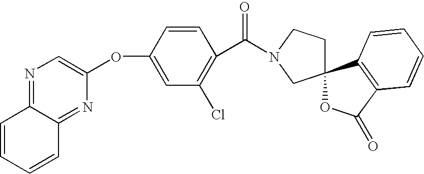

This compound was prepared using procedures analogous to example 4. LC-MS: 472.1/474.1 (M+H)⁺.

### Example 17

### (1R)-1′-[2-Chloro-4-(pyrimidin-2-yloxy)benzoyl-3H-spiro[2-benzofuran-1,3′-pyrrolidin]-3-one

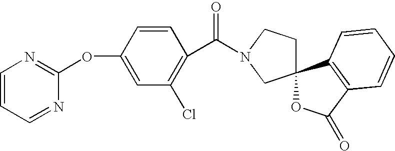

This compound was prepared using procedures analogous to example 4. LC-MS: 422.1/424.1 (M+H)⁺.

### Example 18

### (1R)-1′-{4-[(4-Amino-5-fluoropyrimidin-2-yl)oxy-2-chlorobenzoyl-3H-spiro[2-benzofuran-1,3′-pyrrolidin]-3-one

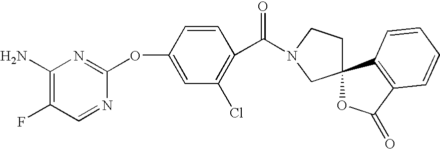

This compound was prepared using procedures analogous to example 4. LC-MS: 455.1/457.1 (M+H)⁺.

### Example 19

### (1R)-1′-{2-Chloro-4-[(4-chloropyrimidin-2-yl)oxy]benzoyl}-3H-spiro[2-benzofuran-1,3′-pyrrolidin]-3-one

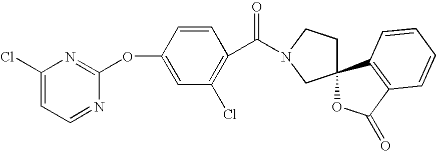

This compound was prepared using procedures analogous to example 4. LC-MS: 456.0/458.0 (M+H)⁺.

### Example 20

### (1R)-1′-{2-Chloro-4-[(6-chloro-9H-purin-2-yl)oxy]benzoyl}-3H-spiro[2-benzofuran-1,3′-pyrrolidin]-3-one

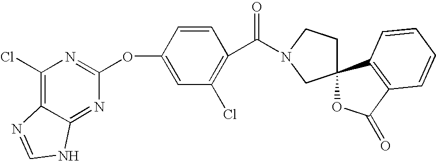

This compound was prepared using procedures analogous to example 4. LC-MS: 496.0/498.0 (M+H)⁺.

### Example 21

### (1R)-1′-{2-Chloro-4-[(6-chloropyrazin-2-yl)oxy]benzoyl}-3H-spiro[2-benzofuran-1,3′-pyrrolidin]-3-one

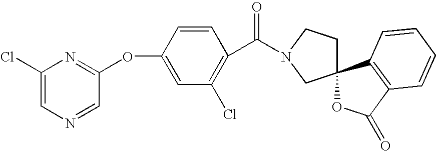

This compound was prepared using procedures analogous to example 4. LC-MS: 496.0/498.0 (M+H)⁺.

### Example 22

### (1R)-1′-(4-Bromo-2-chlorobenzoyl)-3H-spiro[2-benzofuran-1,3′-pyrrolidin]-3-one

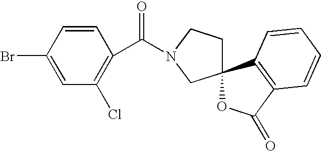

This compound was prepared using procedures analogous to example 1. LC-MS: 406.0/407.9 (M+H)⁺.

### Example 23

### (1R)-1′-[2-Chloro-5-(pyrazin-2-yloxy)benzoyl]-3H-spiro[2-benzofuran-1,3′-pyrrolidin]-3-one

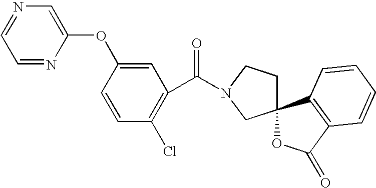

This compound was prepared using procedures analogous to example 4. LC-MS: 422.0 (M+H)⁺.

### Example 24

### (1R)-1′-(4-Aminobenzoyl)-3H-spiro[2-benzofuran-1,3′-pyrrolidin]-3-one

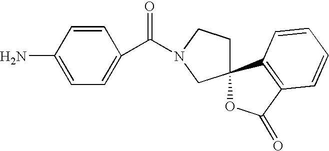

This compound was prepared using procedures analogous to example 1. LC-MS: 309.1 (M+H)⁺.

### Example 25

### 4-Fluoro-N-{4-[(3-oxo-1′H,3H-spiro[2-benzofuran-1,3′-pyrrolidin]-1′-yl)carbonyl]phenyl}benzamide

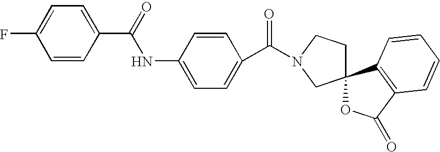

This compound was prepared using procedures analogous to example 1. LC-MS: 431.1 (M+H)⁺.

### Example 26

### tert-Butyl 4-(3-chloro-4-{[(1R)-3-oxo-1′H,3H-spiro[2-benzofuran-1,3′-pyrrolidin]-1′-yl]carbonyl}phenyl)piperazine-1-carboxylate

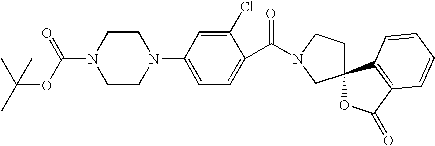

A mixture of (1R)-1′-(4-bromo-2-chlorobenzoyl)-3H-spiro[2-benzofuran-1,3′-pyrrolidin]-3-one (407 mg, 0.00100 mol, prepared as example 22), tert-butyl piperazine-1-carboxylate (224 mg, 0.00120 mol), sodium tert-butoxide (231 mg, 0.00240 mol), palladium acetate (6.74 mg, 0.0000300 mol) and 2-(di-tert-butylphosphino)biphenyl (8.95 mg, 0.0000300 mol) was degassed and then charged with nitrogen. To the mixture was added 1,4-dioxane (4.0 mL, 0.051 mol) and the resulting mixture was refluxed for 16 h. The mixture was poured into ice-water and acidified with 1 N HCl (the pH was adjusted to −3). The product was extracted with ethyl acetate, washed with water and brine, dried over Na₂SO₄, filtered, and concentrated under reduced pressure. The product was purified by CombiFlash eluting with CH₂Cl₂/methanol (max. MeOH 5%). LC-MS: 513.1 (M+H)⁺.

### Example 27

### (1R)-1′-(2-Chloro-4-piperazin-1-ylbenzoyl)-3H-spiro[2-benzofuran-1,3′-pyrrolidin]-3-one dihydrochloride

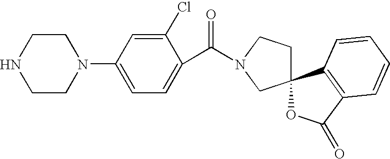

tert-Butyl 4-(3-chloro-4-{[(1R)-3-oxo-1′H,3H-spiro[2-benzofuran-1,3′-pyrrolidin]-1′-yl]carbonyl}phenyl)piperazine-1-carboxylate (0.490 g, 0.000997 mol, prepared as example 26) in methanol (0.5 mL) was treated with hydrogen chloride in 1,4-dioxane (4.0 M, 1.00 mL) at rt for 3 h. The volatiles were removed in-vacuo and the residue was dried under reduced pressure to afford the desired product. LC-MS: 412.2 (M+H)⁺.

### Example 28

### (1R)-1′-[4-(4-Acetylpiperazin-1-yl)-2-chlorobenzoyl]-3H-spiro[2-benzofuran-1,3′-pyrrolidin]-3-one

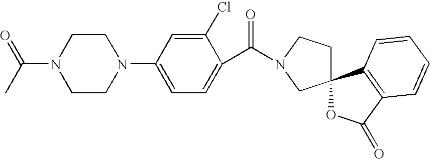

Acetyl chloride (3.2 uL, 0.000045 mol) was added to a mixture of (1R)-1′-(2-chloro-4-piperazin-1-ylbenzoyl)-3H-spiro[2-benzofuran-1,3′-pyrrolidin]-3-one (7.5 mg, 0.000018 mol, prepared as example 28) and N,N-diisopropylethylamine (9.5 uL, 0.000054 mol) in acetonitrile (0.5 mL, 0.01 mol). After stirring at rt for 30 min., the crude reaction mixture was purified by prep-LCMS to afford the desired product. LC-MS: 454.2 (M+H)⁺.

### Example 29

### (1R)-1′-[2-Chloro-4-(4-propionylpiperazin-1-yl)benzoyl]-3H-spiro[2-benzofuran-1,3′-pyrrolidin]-3-one

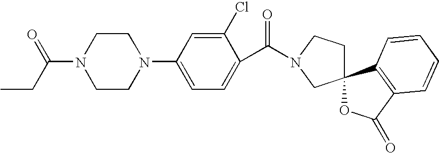

This compound was prepared using procedures analogous to example 28. LC-MS: 468.2 (M+H)⁺.

### Example 30

### (1R)-1′-[4-(4-Butyrylpiperazin-1-yl)-2-chlorobenzoyl]-3H-spiro[2-benzofuran-1,3′-pyrrolidin]-3-one

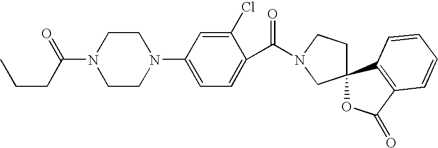

This compound was prepared using procedures analogous to example 28. LC-MS: 482.2 (M+H)⁺.

### Example 31

### (1R)-1′-{2-Chloro-4-[4-(cyclopropylcarbonyl)piperazin-1-yl]benzoyl}-3H-spiro[2-benzofuran-1,3′-pyrrolidin]-3-one

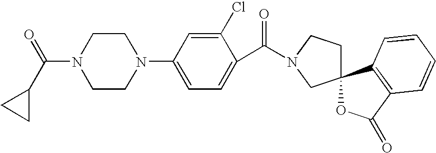

This compound was prepared using procedures analogous to example 28. LC-MS: 480.2 (M+H)⁺.

### Example 32

### Methyl 4-(3-chloro-4-{[(1R)-3-oxo-1′H,3H-spiro[2-benzofuran-1,3′-pyrrolidin]-1′-yl]carbonyl}phenyl)piperazine-1-carboxylate

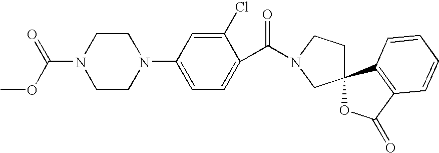

This compound was prepared using procedures analogous to example 28. LC-MS: 470.2 (M+H)⁺.

### Example 33

### Ethyl 4-(3-chloro-4-{[(1R)-3-oxo-1′H,3H-spiro[2-benzofuran-1,3′-pyrrolidin]-1′-yl]carbonyl}phenyl)piperazine-1-carboxylate

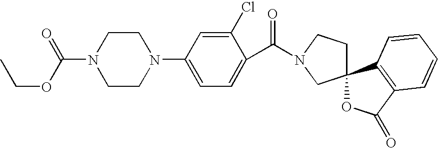

This compound was prepared using procedures analogous to example 28. LC-MS: 484.2 (M+H)⁺.

### Example 34

### Propyl 4-(3-chloro-4-{[(1R)-3-oxo-1′H,3H-spiro[2-benzofuran-1,3′-pyrrolidin-1′-yl]carbonyl}phenyl)piperazine-1-carboxylate

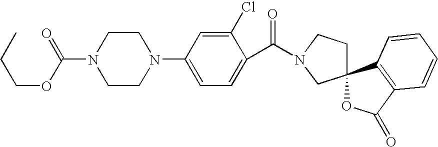

This compound was prepared using procedures analogous to example 28. LC-MS: 498.2 (M+H)⁺.

### Example 35

### Isobutyl 4-(3-chloro-4-{[(1R)-3-oxo-1′H,3H-spiro[2-benzofuran-1,3′-pyrrolidin]-1′-yl]carbonyl}phenyl)piperazine-1-carboxylate

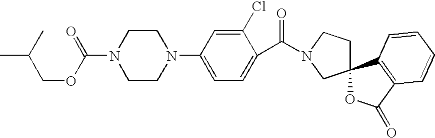

This compound was prepared using procedures analogous to example 28. LC-MS: 512.2 (M+H)⁺.

### Example 36

### (1R)-1′-{2-Chloro-4-[4-(ethylsulfonyl)piperazin-1-yl]benzoyl}-3H-spiro[2-benzofuran-1,3′-pyrrolidin]-3-one

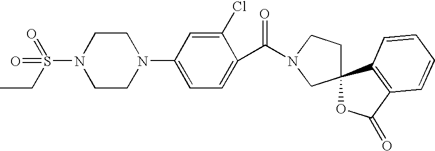

This compound was prepared using procedures analogous to example 28. LC-MS: 504.1 (M+H)⁺.

### Example 37

### tert-Butyl 4-(3-methyl-4-{[(1R)-3-oxo-1′H,3H-spiro[2-benzofuran-1,3′-pyrrolidin]-1′-yl]carbonyl}phenyl)piperazine-1-carboxylate

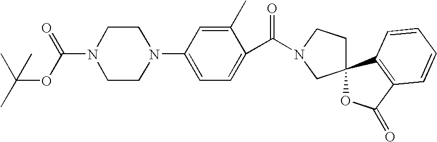

This compound was prepared using procedures analogous to example 26. LC-MS: 492.2 (M+H)⁺.

### Example 38

### (1R)-1′-(2-Methyl-4-piperazin-1-ylbenzoyl)-3H-spiro[2-benzofuran-1,3′-pyrrolidin]-3-one dihydrochloride

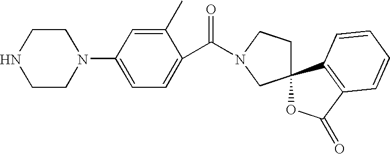

This compound was prepared using procedures analogous to example 27. LC-MS: 392.2 (M+H)⁺.

### Example 39

### Methyl 4-(3-methyl-4-{[(1R)-3-oxo-1′H,3H-spiro[2-benzofuran-1,3′-pyrrolidin]-1′-yl]carbonyl}phenyl)piperazine-1-carboxylate

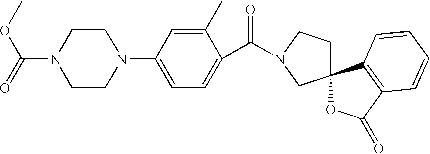

This compound was prepared using procedures analogous to example 28. LC-MS: 450.2 (M+H)⁺.

### Example 40

### Ethyl 4-(3-methyl-4-([(1R)-3-oxo-1′H,3H-spiro[2-benzofuran-1,3′-pyrrolidin]-1′-yl]carbonyl}phenyl)piperazine-1-carboxylate

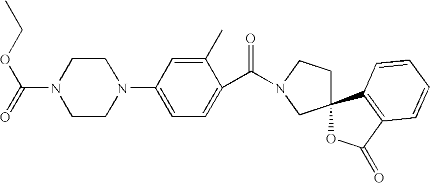

This compound was prepared using procedures analogous to example 28. LC-MS: 464.2 (M+H)⁺.

### Example 41

### Propyl 4-(3-methyl-4-{[(1R)-3-oxo-1′H,3H-spiro[2-benzofuran-1,3′-pyrrolidin]-1′-yl]carbonyl}phenyl)piperazine-1-carboxylate

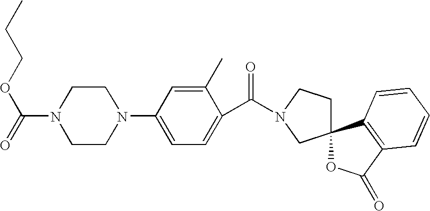

This compound was prepared using procedures analogous to example 28. LC-MS: 478.2 (M+H)⁺.

### Example 42

### Prop-2-yn-1-yl 4-(3-methyl-4-{[(1R)-3-oxo-1′H,3H-spiro[2-benzofuran-1,3′-pyrrolidin]-1′-yl]carbonyl}phenyl)piperazine-1-carboxylate

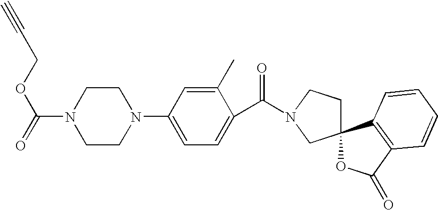

This compound was prepared using procedures analogous to example 28. LC-MS: 474.2 (M+H)⁺.

### Example 43

### Isopropyl 4-(3-methyl-4-{[(1R)-3-oxo-1′H,3H-spiro[2-benzofuran-1,3′-pyrrolidin]-1′-yl]carbonyl}phenyl)piperazine-1-carboxylate

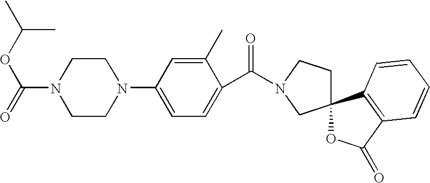

This compound was prepared using procedures analogous to example 28. LC-MS: 478.2 (M+H)⁺.

### Example 44

### Isobutyl 4-(3-methyl-4-{[(1R)-3-oxo-1′H,3H-spiro[2-benzofuran-1,3′-pyrrolidin]-1′-yl]carbonyl}phenyl)piperazine-1-carboxylate

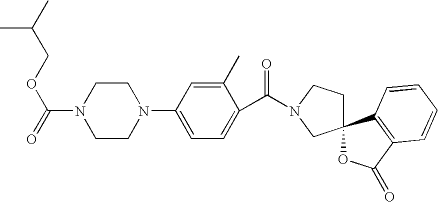

This compound was prepared using procedures analogous to example 28. LC-MS: 492.2 (M+H)⁺.

### Example 45

### (1R)-1′-{2-Methyl-4-[4-(methylsulfonyl)piperazin-1-yl]benzoyl}-3H-spiro[2-benzofuran-1,3′-pyrrolidin]-3-one

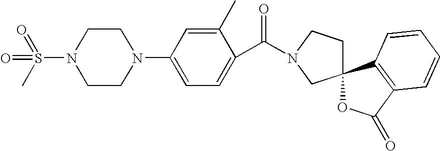

This compound was prepared using procedures analogous to example 28. LC-MS: 470.2 (M+H)⁺.

### Example 46

### (1R)-1′-{4-[4-(Ethylsulfonyl)piperazin-1-yl]-2-methylbenzoyl}-3H-spiro[2-benzofuran-1,3′-pyrrolidin]-3-one

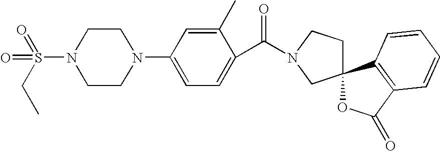

This compound was prepared using procedures analogous to example 28. LC-MS: 484.2 (M+H)⁺.

### Example 47

### (1R)-1′-[4-(4-Acetylpiperazin-1-yl)-2-methylbenzoyl]-3H-spiro[2-benzofuran-1,3′-pyrrolidin]-3-one

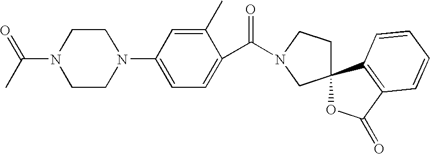

This compound was prepared using procedures analogous to example 28. LC-MS: 434.2 (M+H)⁺.

### Example 48

### (1R)-1′-[2-Methyl-4-(4-propionylpiperazin-1-yl)benzoyl]-3H-spiro[2-benzofuran-1,3′-pyrrolidin]-3-one

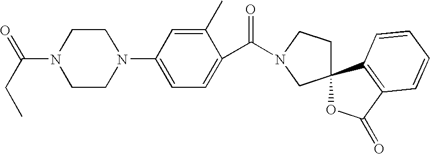

This compound was prepared using procedures analogous to example 28. LC-MS: 448.2 (M+H)⁺.

### Example 49

### (1R)-1′-[4-(4-Isobutyrylpiperazin-1-yl)-2-methylbenzoyl]-3H-spiro[2-benzofuran-1,3′-pyrrolidin]-3-one

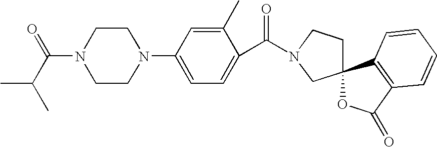

This compound was prepared using procedures analogous to example 28. LC-MS: 462.2 (M+H)⁺.

### Example 50

### (1R)-1′-{4-[4-(Cyclopropylcarbonyl)piperazin-1-yl]-2-methylbenzoyl-3H-spiro[2-benzofuran-1,3′-pyrrolidin]-3-one

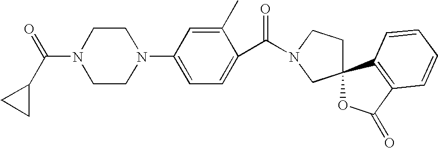

This compound was prepared using procedures analogous to example 28. LC-MS: 460.2 (M+H)⁺.

### Example 51

### (1R)-1′-[2-Chloro-4-(9H-purin-9-yl)benzoyl]-3H-spiro[2-benzofuran-1,3′-pyrrolidin]-3-one

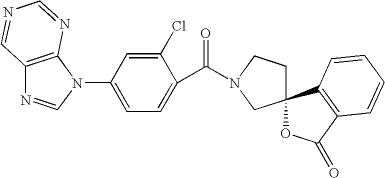

To a solution of (1R)-1′-(4-bromo-2-chlorobenzoyl)-3H-spiro[2-benzofuran-1,3′-pyrrolidin]-3-one (30.0 mg, 0.0000738 mol, prepared as example 22), in 1,4-dioxane (0.268 mL, 0.00344 mol) were added (1S,2S)-N,N′-dimethylcyclohexane-1,2-diamine (2.1 mg, 0.000015 mol), copper(I) iodide (1.4 mg, 0.0000074 mol), 9H-purine (13 mg, 0.00011 mol) and potassium carbonate (0.0214 g, 0.000155 mol). The reaction mixture was heated to reflux and stirred for 16 h. The crude reaction mixture was purified by prep-HPLC to afford the desired procuct. LC-MS: 446.1 (M+H)⁺.

### Example 52

### (1R)-1′-[4-(2-Oxopyrrolidin-1-yl)benzoyl]-3H-spiro[2-benzofuran-1,3′-pyrrolidin]-3-one

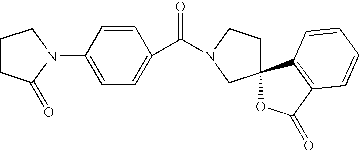

This compound was prepared using procedures analogous to example 51. LC-MS: 377.2 (M+H)⁺.

### Example 53

### (1R)-1′-[4-(2-Oxo-1,3-oxazolidin-3-yl)benzoyl]-3H-spiro[2-benzofuran-1,3′-pyrrolidin]-3-one

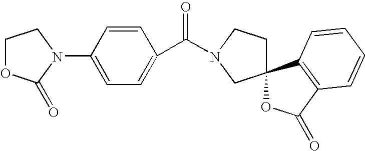

This compound was prepared using procedures analogous to example 51. LC-MS: 379.1 (M+H)⁺.

### Example 54

### (1R)-1′-[2-Chloro-4-(3-methyl-1H-pyrazol-1-yl)benzoyl]-3H-spiro[2-benzofuran-1,3′-pyrrolidin]-3-one

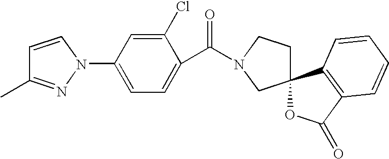

This compound was prepared using procedures analogous to example 51. LC-MS: 408.1 (M+H)⁺.

### Example 55

### (1R)-1′-[2-Chloro-4-(1H-pyrazol-1-yl)benzoyl]-3H-spiro[2-benzofuran-1,3′-pyrrolidin]-3-one

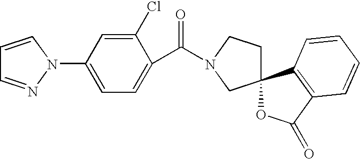

This compound was prepared using procedures analogous to example 51. LC-MS: 394.1 (M+H)⁺.

### Example 56

### (1R)-1′-(4-Morpholin-4-ylbenzoyl)-3H-spiro[2-benzofuran-1,3′-pyrrolidin]-3-one

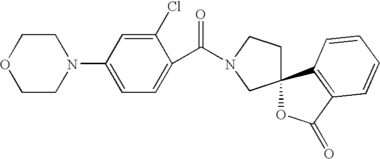

This compound was prepared using procedures analogous to example 1. LC-MS: 379.1 (M+H)⁺.

### Example 57

### tert-Butyl 4-(3-chloro-4-{[(1R)-3-oxo-1′H,3H-spiro[2-benzofuran-1,3′-pyrrolidin]-1′-yl]carbonyl}phenyl)-3,6-dihydropyridine-1(2H)-carboxylate

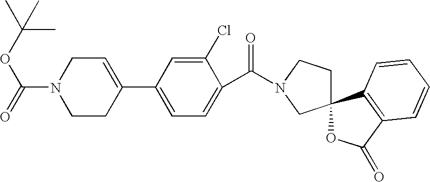

### Step 1. tert-butyl 4-([(trifluoromethyl)sulfonyl]oxy}-3,6-dihydropyridine-1(2H)-carboxylate

To a solution of tert-butyl 4-oxo-1-piperidinecarboxylate (10.50 g, 0.05270 mol) in tetrahydrofuran (200.0 mL, 2.466 mol) at −78° C., under an atmosphere of nitrogen, was added 1.000 M of lithium hexamethyldisilazide in tetrahydrofuran (55.96 mL). After stirring at −78° C. for 1 h, solid N-phenylbis(trifluoromethanesulphonimide) (20.00 g, 0.05598 mol) was added. The reaction mixture was stirred at −78° C. for 2 h, then was allowed to warm to rt gradually and stirred for additional 16 h. The volatiles were removed under reduced pressure and the residue was diluted with ether. The mixture was washed with 1 N HCl, 1 N NaOH and brine, successively. The organic layer was then dried and evaporated to dryness. The residue was applied on a silica gel column, eluting with 0 to 20% ethyl acetate in hexane to provide the desired enol triflate. LC-MS (ESI): 232.0 (M-Boc)⁺.

### Step 2. tert-butyl 4-(4,4,5,5-tetramethyl-1,3,2-dioxaborolan-2-yl)-3,6-dihydropyridine-1(2H)-carboxylate

A 1 L flask was charged with 4,4,5,5,4′,4′,5′,5′-octamethyl-[2,2′]bi[[1,3,2] dioxaborolanyl] (13.0 g, 0.0511 mol) [bis(pinacolato)diborane], sodium acetate (11.4 g, 0.139 mol), {[1,1′-bis(diphenylphosphino)ferrocene]dichloropalladium(II), complex with dichloromethane (1:1)} (1.1 g, 0.0014 mol, [PdCl₂dppf],) 1,1′-bis(diphenylphosphino)ferrocene (0.77 g, 0.0014 mol, [dppf]) and 1,4-dioxane (100 mL). A solution of tert-butyl 4-{[(trifluoromethyl)sulfonyl]oxy}-3,6-dihydropyridine-1(2H)-carboxylate (15.4 g, 0.0465 mol) in 1,4-dioxane (200 mL) was added to the above mixture under an atmosphere of nitrogen. The resulting mixture was stirred under an atmosphere of nitrogen at 80° C. overnight. The reaction mixture was quenched by an addition of water and then extracted with EtOAc (3×). The combined organic layers were washed with water, brine, dried over MgSO₄ and concentrated in-vacuo. The crude product was purified by flash column chromatography eluting with 0-10% EtOAc in hexane to afford the product as a off-white wax-like solid. The product structure was confirmed by ¹H NMR spectroscopy.

### Step 3

To a solution of tert-butyl 4-(4,4,5,5-tetramethyl-1,3,2-dioxaborolan-2-yl)-3,6-dihydropyridine-1(2H)-carboxylate (0.10 g, 0.00032 mol) and (1R)-1′-(4-bromo-2-chlorobenzoyl)-3H-spiro[2-benzofuran-1,3′-pyrrolidin]-3-one (0.16 g, 0.00039 mol, prepared as example 22) in N,N-dimethylformamide (1.0 mL, 0.013 mol) were added [1,1′-bis(diphenylphosphino)ferrocene]dichloropalladium(II), complex with dichloromethane (1:1) (20 mg, 0.00002 mol) and potassium carbonate (0.13 g, 0.00097 mol), and the mixture was heated at 100° C. under nitrogen for 16 h. The product was filtered through a short plug of silica gel and washed with ethyl acetate. The volatiles were removed and the crude product was purified by CombiFlash eluting with hexane/EtOAc (max. EtOAc 60%). LC-MS: 453.1/455.1 (M+H-Bu(56))⁺.

### Example 58

### (1R)-1′-[2-Chloro-4-(1,2,3,6-tetrahydropyridin-4-yl)benzoyl]-3H-spiro[2-benzofuran-1,3′-pyrrolidin]-3-one

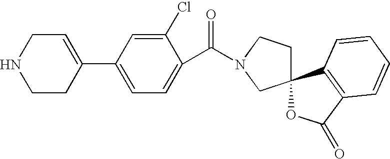

To a solution of tert-butyl 4-(3-chloro-4-{[(1R)-3-oxo-1′H,3H-spiro[2-benzofuran-1,3′-pyrrolidin]-1′-yl]carbonyl}phenyl)-3,6-dihydropyridine-1 (2H)-carboxylate (0.10 g, 0.00020 mol, prepared as example 57) in methylene chloride (0.2 mL, 0.003 mol) was added 4.0 M of hydrogen chloride in 1,4-dioxane (2.0 mL), and the resultant mixture was stirred at rt for 2 h. The mixture was diluted with ether and the precipitate formed was filtered and dried to afford the desired product. LC-MS: 409.1/411.1 (M+H)⁺.

### Example 59

### Methyl 4-(3-chloro-4-{[(1R)-3-oxo-1′H,3H-spiro[2-benzofuran-1,3′-pyrrolidin]-1′-yl]carbonylphenyl)-3,6-dihydropyridine-1(2H)-carboxylate

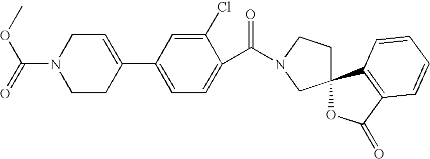

Methyl chloroformate (0.010 mL, 0.0001 mol) was added to a solution of (1R)-1′-[2-chloro-4-(1,2,3,6-tetrahydropyridin-4-yl)benzoyl]-3H-spiro[2-benzofuran-1,3′-pyrrolidin]-3-one (19.6 mg, 0.0000479 mol, prepared as example 58) and N,N-diisopropylethylamine (28 μL, 0.00016 mol) in methylene chloride (0.8 mL, 0.01 mol), and the mixture was stirred for 1 h. The mixture was acidified by adding TFA and the volatiles were removed to afford a residue that was purified by prep-HPLC. LC-MS: 467.1/469.1 (M+H)⁺.

### Example 60

### (1R)-1′-[2-Chloro-4-(1-isobutyryl-1,2,3,6-tetrahydropyridin-4-yl)benzoyl]-3H-spiro[2-benzofuran-1,3′-pyrrolidin]-3-one

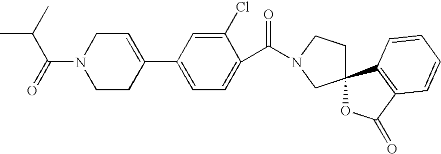

This compound was prepared using procedures analogous to example 59. LC-MS: 479.2/481.2 (M+H)⁺.

### Example 61

### (1R)-1′-[2-Chloro-4-(1-isobutyrylpiperidin-4-yl)benzoyl]-3H-spiro[2-benzofuran-1,3′-pyrrolidin]-3-one

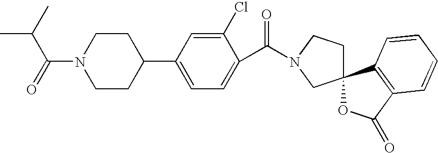

Pd/C (5 wt %, Degussa type F101 ra/w, Aldrich # 330159, 1.0 mg) was added to a solution of (1R)-1′-[2-chloro-4-(1-isobutyryl-1,2,3,6-tetrahydropyridin-4-yl)benzoyl]-3H-spiro[2-benzofuran-1,3′-pyrrolidin]-3-one (4.0 mg, 0.0000084 mol, prepared as example 60) in methanol (1.0 mL, 0.025 mol), and the reaction mixture was stirred under a hydrogen balloon for 2 h (LC-MS indicated completion). The reaction mixture was filtered through Celite and the filtrate was concentrated to afford the desired product. LC-MS: 481.2 (M+H)⁺.

### Example 62

### Methyl 4-(4-{[(1R)-3-oxo-1′H,3H-spiro[2-benzofuran-1,3′-pyrrolidin]-1′-yl]carbonyl}phenyl)piperidine-1-carboxylate

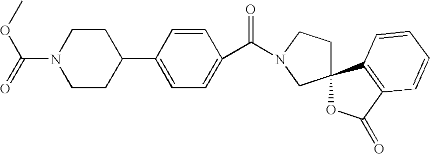

### Step 1. tert-butyl 4-(4-([(1R)-3-oxo-1′H,3H-spiro[2-benzofuran-1,3′-pyrrolidin]-1′-yl]carbonyl}phenyl)piperidine-1-carboxylate

Pd on carbon (20 mg, 10%) was added to a solution of tert-butyl 4-(4-{[(1R)-3-oxo-1′H,3H-spiro[2-benzofuran-1,3′-pyrrolidin]-1′-yl]carbonyl}phenyl)-3,6-dihydropyridine-1 (2H)-carboxylate (0.15 g, 0.00032 mol, prepared using procedures analogous to those used for the synthesis of example 57) in methanol (5.0 mL, 0.12 mol) and DMF (0.5 mL), and the mixture was stirred under a hydrogen balloon for 1 h. The reaction mixture was filtered and the volatiles of the filtrate was removed to afford the desired product. LC-MS: 499.2 (M+Na)⁺.

### Step 2. (1R)-1′-(4-piperidin-4-ylbenzoyl)-3H-spiro[2-benzofuran-1,3′-pyrrolidin]-3-one hydrochloride

This compound was prepared using procedures analogous to example 58. LC-MS: 377.2 (M+H)⁺.

### Step 3. Methyl 4-(4-([(]R)-3-oxo-1′H,3H-spiro[2-benzofuran-1,3′-pyrrolidin]-1′-yl]carbonylyphenyl)piperidine-1-carboxylate

This compound was prepared using procedures analogous to example 59. LC-MS: 435.2 (M+H)⁺.

### Example 63

### (1R)-1′-(5-Bromo-2-chlorobenzoyl)-3H-spiro[2-benzofuran-1,3′-pyrrolidin-3-one

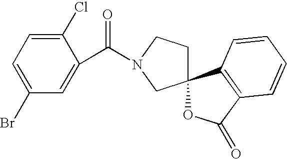

This compound was prepared using procedures analogous to example 1. LC-MS: 406.0/407.9 (M+H)⁺.

### Example 64

### (1R)-1′-(2-Chloro-4-hydroxybenzoyl)-3H-spiro[2-benzofuran-1,3′-pyrrolidin]-3-one

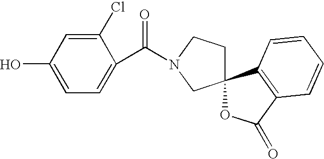

This compound was prepared using procedures analogous to example 1. LC-MS: 344.1/346.1 (M+H)⁺.

### Example 65

### (1R)-1′-(2-Chloro-5-hydroxybenzoyl)-3H-spiro[2-benzofuran-1,3′-pyrrolidin]-3-one

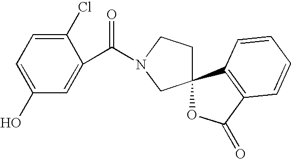

This compound was prepared using procedures analogous to example 1. LC-MS: 344.0/346.0 (M+H)⁺.

### Example 66

### (1R)-1′-[2-Chloro-4-(5-methoxypyridin-3-yl)benzoyl]-3H-spiro 12-benzofuran-1,3′-pyrrolidin]-3-one

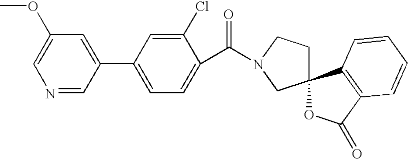

A solution of sodium carbonate (21.2 mg, 0.000200 mol) in water (0.20 mL) was added to a mixture of (1R)-1′-(4-bromo-2-chlorobenzoyl)-3H-spiro[2-benzofuran-1,3′-pyrrolidin]-3-one (40.7 mg, 0.000100 mol, prepared as example 22), (5-methoxypyridin-3-yl)boronic acid (18.4 mg, 0.000120 mol) and tetrakis(triphenylphosphine)palladium(0) (3.5 mg, 0.0000030 mol) in toluene (200.0 uL, 0.001878 mol) and ethanol (100.00 uL, 0.0017127 mol). The resulting mixture was irradiated by microwaves at 120° C. for 20 min. Ethyl acetate (5 mL) was added and the mixture was washed with water and brine. The organic layer was dried over Na₂SO₄, filtered and concentrated under reduced pressure. The residue was dissolved in DMF and purified by prep-HPLC to afford the desired product. LC-MS: 435.2 (M+H)⁺.

### Example 67

### (1R)-1′-2-Chloro-4-(3,5-dimethylisoxazol-4-yl)benzoyl]-3H-spiro[2-benzofuran-1,3′-pyrrolidin]-3-one

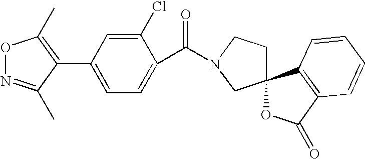

This compound was prepared using procedures analogous to example 66. LC-MS: 423.1 (M+H)⁺.

### Example 68

### (1R)-1′-2-Chloro-4-(6-methoxypyridin-3-yl)benzoyl]-3H-spiro[2-benzofuran-1,3′-pyrrolidin]-3-one

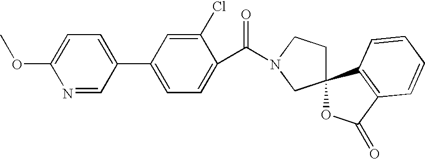

This compound was prepared using procedures analogous to example 66. LC-MS: 435.2 (M+H)⁺.

### Example 69

### (1R)-1′-(2-Chloro-4-pyrimidin-5-ylbenzoyl)-3H-spiro[2-benzofuran-1,3′-pyrrolidin]-3-one

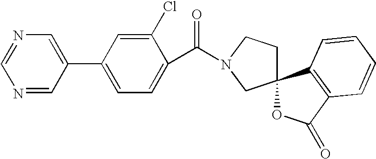

This compound was prepared using procedures analogous to example 66. LC-MS: 406.2 (M+H)⁺.

### Example 70

### (1R)-1′-(2-Chloro-4-pyrazin-2-ylbenzoyl)-3H-spiro[2-benzofuran-1,3′-pyrrolidin]-3-one

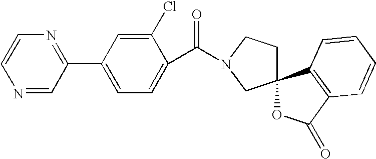

This compound was prepared using procedures analogous to those described for the synthesis of example 66 with the exception that the organometallic coupling partners were reversed: 2-chloropyrazine was coupled to (1R)-1′-[2-chloro-4-(4,4,5,5-tetramethyl-1,3,2-dioxaborolan-2-yl)benzoyl]-3H-spiro[2-benzofuran-1,3′-pyrrolidin]-3-one which was prepared by using a procedure analogous to that described for the synthesis of example 57, step 2 {starting from (1R)-1′-(4-bromo-2-chlorobenzoyl)-3H-spiro[2-benzofuran-1,3′-pyrrolidin]-3-one (example 22)}. LC-MS: 406.1 (M+H)⁺.

### Example 71

### 3′-Chloro-4′-{[(1R)-3-oxo-1′H,3H-spiro[2-benzofuran-1,3′-pyrrolidin]-1′-yl]carbonyl}biphenyl-3-carbonitrile

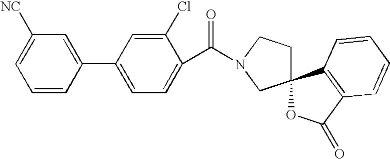

This compound was prepared using procedures analogous to example 66. LC-MS: 429.1 (M+H)⁺.

### Example 72

### (1R)-1′-[4-(1,3-Benzodioxol-5-yl)-2-chlorobenzoyl]-3H-spiro[2-benzofuran-1,3′-pyrrolidin]-3-one

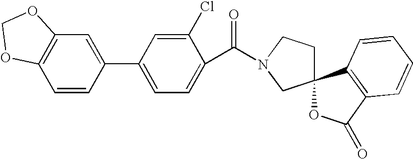

This compound was prepared using procedures analogous to example 66. LC-MS: 448.1 (M+H)⁺.

### Example 73

### (1R)-1′-{[3-Chloro-3′-(hydroxymethyl)biphenyl-4-yl]carbonyl}-3H-spiro[2-benzofuran-1,3′-pyrrolidin]-3-one

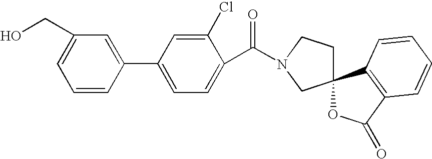

This compound was prepared using procedures analogous to example 66. LC-MS: 434.1 (M+H)⁺.

### Example 74

### 3′-Chloro-4′-{[(1R)-3-oxo-1′H,3H-spiro[2-benzofuran-1,3′-pyrrolidin]-1′-yl]carbonyl}biphenyl-3-carboxamide

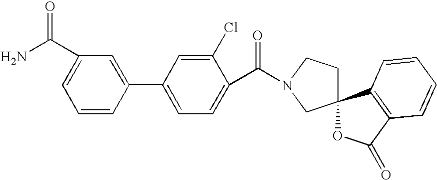

This compound was prepared using procedures analogous to example 66. LC-MS: 447.1 (M+H)⁺.

### Example 75

### (1R)-1′-[(3′-Amino-3-chlorobiphenyl-4-yl)carbonyl]-3H-spiro[2-benzofuran-1,3′-pyrrolidin]-3-one

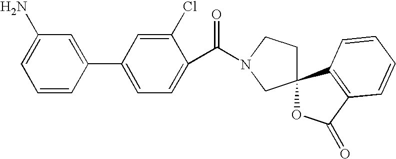

This compound was prepared using procedures analogous to example 66. LC-MS: 419.1 (M+H)⁺.

### Example 76

### Methyl (3′-chloro-4′-{[(1R)-3-oxo-1′H,3H-spiro[2-benzofuran-1,3′-pyrrolidin]-1′-yl]carbonyl}biphenyl-3-yl)carbamate

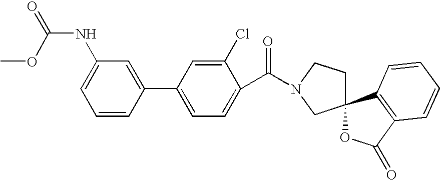

This compound was prepared using procedures analogous to example 59 starting with (1R)-1′-[(3′-Amino-3-chlorobiphenyl-4-yl)carbonyl]-3H-spiro[2-benzofuran-1,3′-pyrrolidin]-3-one (example 75). LC-MS: 477.0 (M+H)⁺.

### Example 77

### Propyl (3′-chloro-4′-{[(1R)-3-oxo-1′H,3H-spiro[2-benzofuran-1,3′-pyrrolidin]-1′-yl]carbonyl}biphenyl-3-yl)carbamate

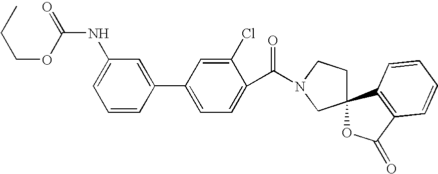

This compound was prepared using procedures analogous to example 76. LC-MS: 505.1 (M+H)⁺.

### Example 78

### Isobutyl (3′-chloro-4′-{[(1R)-3-oxo-1′H,3H-spiro[2-benzofuran-1,3′-pyrrolidin]-1′-yl]carbonyl}biphenyl-3-yl)carbamate

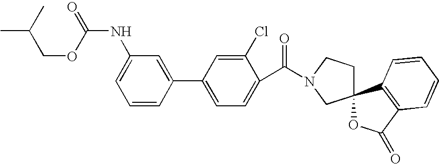

This compound was prepared using procedures analogous to example 76. LC-MS: 519.0 (M+H)⁺.

### Example 79

### (1R)-1′-{[3-Chloro-3′-(2-oxopyrrolidin-1-yl)biphenyl-4-yl]carbonyl}-3H-spiro[2-benzofuran-1,3′-pyrrolidin]-3-one

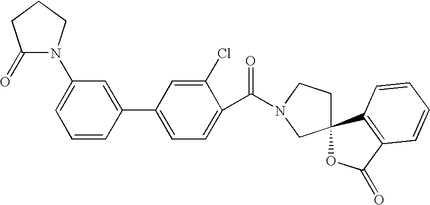

(1R)-1′-[(3′-amino-3-chlorobiphenyl-4-yl)carbonyl]-3H-spiro[2-benzofuran-1,3′-pyrrolidin]-3-one (10 mg, 0.00002 mol; example 75) was dissolved in THF (0.5 mL) and to this were added 4-dimethylaminopyridine (0.0044 g, 0.000036 mol) and 4-bromobutanoyl chloride (3.6 μL, 0.000031 mol). The mixture was stirred for 3 h at rt followed by an addition of NaH (29 mg, 60% by wt., oil dispersion) (resulting in effervescence and the solution turning yellow). After stirring for 2 h the reaction mixture was quenched by an addition of H₂O followed by an addition of saturated NH₄Cl. The solution was then diluted with EtOAc (15 mL) and H₂O (5 mL) and the resulting layers were separated. The aqueous layer was extracted with EtOAc (3×5 mL) and the combined organic layers were washed with H₂O (5 mL) then brine (2×5 mL), dried (over NaSO₄), filtered, and concentrated in-vacuo. The crude residue was purified by prep-HPLC to afford the desired product. LC-MS: 487.1 (M+H)⁺.

### Example 80

### (1R)-1′-(1-Naphthoyl)-3H-spiro[2-benzofuran-1,3′-pyrrolidin]-3-one

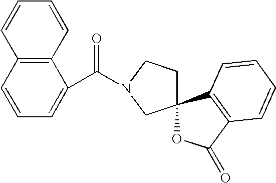

This compound was prepared using procedures analogous to example 1. LC-MS: 344.2 (M+H)⁺.

### Example 81

### (1R)-1′-(2-Naphthoyl)-3H-spiro[2-benzofuran-1,3′-pyrrolidin]-3-one

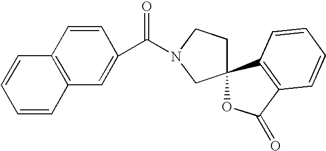

This compound was prepared using procedures analogous to example 1. LC-MS: 344.2 (M+H)⁺.

### Example 82

### (1R)-1′-(3,7-Dihydroxy-2-naphthoyl)-3H-spiro[2-benzofuran-1,3′-pyrrolidin]-3-one

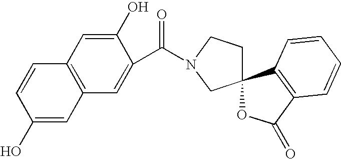

This compound was prepared using procedures analogous to example 1. LC-MS: 376.2 (M+H)⁺.

### Example 83

### (1R)-1′-(6-Methoxy-1-naphthoyl)-3H-spiro[2-benzofuran-1,3′-pyrrolidin]-3-one

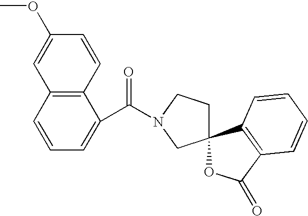

This compound was prepared using procedures analogous to example 1. LC-MS: 374.2 (M+H)⁺.

### Example 84

### (3′-Chloro-4′-{[(1R)-3-oxo-1′H,3H-spiro[2-benzofuran-1,3′-pyrrolidin]-1′-yl]carbonyl}biphenyl-3-yl)methyl dimethylcarbamate

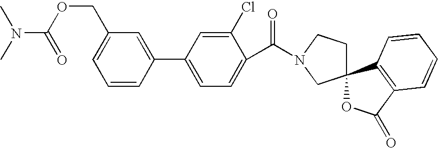

(1R)-1′-{[3-chloro-3′-(hydroxymethyl)biphenyl-4-yl]carbonyl}-3H-spiro[2-benzofuran-1,3′-pyrrolidin]-3-one (8.5 mg, 0.000020 mol; prepared as example 73) was dissolved in DMF (0.5 mL) and to this was added sodium hydride (2.0 mg, 0.000050 mol) (the solution turned yellow upon the addition). After stirring for 5 min. N,N-dimethylcarbamoyl chloride (5.4 mL, 0.000059 mol) was added (the yellow color faded). The reaction mixture was stirred overnight and the LC/MS data indicated that the product was formed. TFA was added to make the pH to −2 and the solution was stirred for 1 h to cyclize the lactone. The crude product was purified by prep-HPLC to afford the desired product. LC/MS: 505.0/507.0 (M+H)⁺.

### Example 85

### 2-Methyl-3-{[(1R)-3-oxo-1′H,3H-spiro[2-benzofuran-1,3′-pyrrolidin]-1′-yl]carbonyl}phenyl acetate

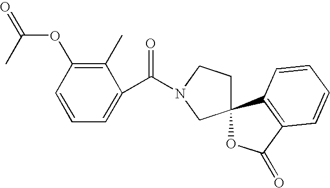

This compound was prepared using procedures analogous to example 1. LC-MS: 366.2 (M+H)⁺.

### Example 86

### Methyl 4-(3-{[(1R)-3-oxo-1′H,3H-spiro[2-benzofuran-1,3′-pyrrolidin]-1′-yl]carbonyl}phenyl)piperidine-1-carboxylate

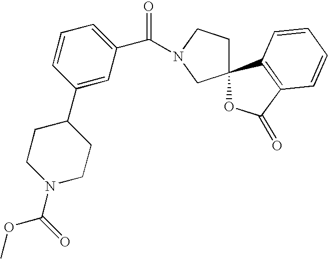

This compound was prepared using procedures analogous to example 1. LC-MS: 435.2 (M+H)⁺.

### Example 87

### tert-Butyl 4-(3-chloro-4-{[(1R)-3-oxo-1′H,3H-spiro[2-benzofuran-1,3′-pyrrolidin]-4′-yl]carbonyl}phenoxy)piperidine-1-carboxylate

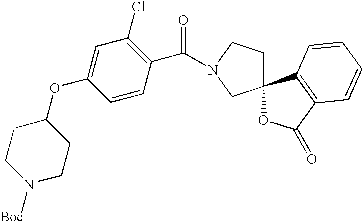

Diethyl azodicarboxylate (15.0 μL, 0.0000953 mol) was added to a mixture of (1R)-1′-(2-chloro-4-hydroxybenzoyl)-3H-spiro[2-benzofuran-1,3′-pyrrolidin]-3-one (13.2 mg, 0.0000384 mol), tert-butyl 4-hydroxypiperidine-1-carboxylate (19.0 mg, 0.0000944 mol) and triphenylphosphine (25.0 mg, 0.0000953 mol) in tetrahydrofuran (1.0 mL, 0.012 mol). After stirring the mixture at rt for 16 h, the crude reaction mixture was diluted with DMF (0.8 mL) and purified by prep-HPLC to afford the desired product. LC-MS: 528.1 (M+H)⁺.

### Example 88

### Methyl 4-(3-methyl-4-{[(1R)-3-oxo-1′H,3H-spiro[2-benzofuran-1,3′-pyrrolidin]-1′-yl]carbonyl}phenyl)-3,6-dihydropyridine-1(2H)-carboxylate

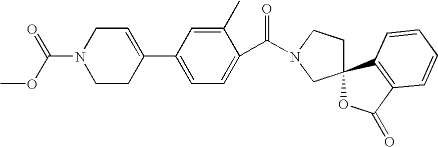

### Step 1. 4-[1-(tert-butoxycarbonyl)-1,2,3,6-tetrahydropyridin-4-yl]-2-methylbenzoic acid

A mixture of 4-bromo-2-methylbenzoic acid (86.02 mg, 0.0004000 mol), tert-butyl 4-(4,4,5,5-tetramethyl-1,3,2-dioxaborolan-2-yl)-3,6-dihydropyridine-1 (2H)-carboxylate (123.7 mg, 0.0004000 mol, prepared in example 57, steps 1 and 2), tetrakis(triphenylphosphine)palladium(0) (14 mg, 0.000012 mol) and sodium carbonate (84.8 mg, 0.000800 mol) in 1,4-dioxane (3.00 mL, 0.0384 mol) and water (0.1 mL) was irradiated by microwaves at 120° C. for 15 min. The mixture was acidified with 1 N HCl (the pH was adjusted to −3.0) and diluted with ethyl acetate (10 mL). The mixture was washed with water and brine. The organic layer was dried over Na₂SO₄, filtered, and concentrated under reduced pressure. The residue was purified by Combiflash eluting with ethyl acetate/hexane to afford the desired product.

### Step 2. tert-butyl 4-(3-methyl-4-{[(1R)-3-oxo-1′H,3H-spiro[2-benzofuran-1,3′-pyrrolidin]-1′-yl]carbonyl}phenyl)-3,6-dihydropyridine-1(2H)-carboxylate

This compound was prepared by using procedures analogous to those used for the synthesis of example 1. LC-MS: 489.3 (M+H)⁺.

### Step 3. Methyl 4-(3-methyl-4-([(1R)-3-oxo-1′H,3H-spiro[2-benzofuran-1,3′-pyrrolidin]-1′-yl]carbonylyphenyl)-3,6-dihydropyridine-1(2H)-carboxylate

The title compound was prepared by using procedures analogous to those used for the synthesis of example 59. LC-MS: 447.2 (M+H)⁺.

### Example 89

### Methyl 4-(3-chloro-4-{[(1R)-3-oxo-1′H,3H-spiro[2-benzofuran-1,3′-pyrrolidin]-1′-yl]carbonyl}phenoxy)piperidine-1-carboxylate

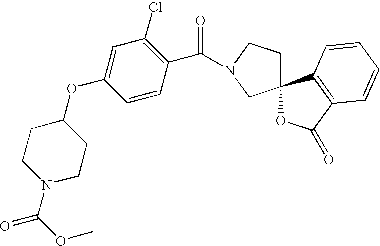

This compound was prepared by using procedures analogous to those used for the synthesis of example 59 starting from tert-butyl 4-(3-chloro-4-{[(1R)-3-oxo-1′H,3H-spiro[2-benzofuran-1,3′-pyrrolidin]-1′-yl]carbonyl}phenoxy)piperidine-1-carboxylate (example 87). LC-MS: 447.2 (M+H)⁺.

### Example 90

### (1R)-1′-{2-Chloro-4-[5-(4-methylpiperazin-1-yl)pyridin-3-yl]benzoyl}-3H-spiro[2-benzofuran-1,3′-pyrrolidin]-3-one

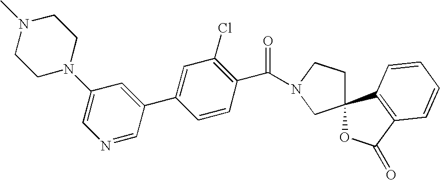

The title compound was prepared by using procedures analogous to those used for the synthesis of example 70. LC-MS: 503.1 (M+H)⁺.

### Example 91

### tert-Butyl 4-(4-methyl-3-{[(1R)-3-oxo-1′H,3H-spiro[2-benzofuran-1,3′-pyrrolidin]-1′-yl]carbonyl}phenyl)piperazine-1-carboxylate

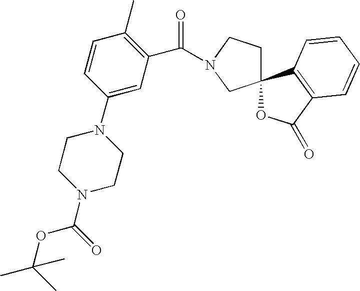

The title compound was prepared by using procedures analogous to those used for the synthesis of example 26. LC-MS: 492.1 (M+H)⁺.

### Example 92

### (1R)-1′-(2-Methyl-5-piperazin-1-ylbenzoyl)-3H-spiro[2-benzofuran-1,3′-pyrrolidin]-3-one

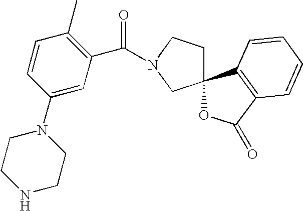

The title compound was prepared by using procedures analogous to those used for the synthesis of example 27. LC-MS: 392.1 (M+H)⁺.

### Example 93

### Methyl 4-(4-methyl-3-{[(1R)-3-oxo-1′H,3H-spiro[2-benzofuran-1,3′-pyrrolidin]-1′-yl]carbonyl}phenyl)piperazine-1-carboxylate

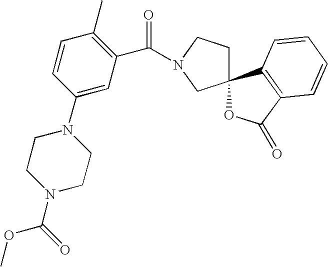

The title compound was prepared by using procedures analogous to those used for the synthesis of example 28. LC-MS: 450.2 (M+H)⁺.

### Example 94

### Ethyl 4-(4-methyl-3-{[(1R)-3-oxo-1′H,3H-spiro[2-benzofuran-1,3′-pyrrolidin]-1′-yl]carbonyl}phenyl)piperazine-1-carboxylate

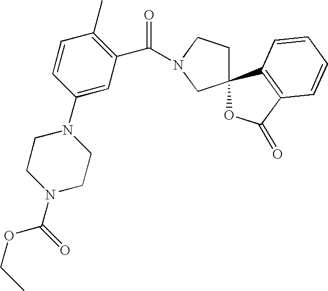

The title compound was prepared by using procedures analogous to those used for the synthesis of example 28. LC-MS: 464.2 (M+H)⁺.

### Example 95

### Propyl 4-(4-methyl-3-{[(1R)-3-oxo-1′H,3H-spiro[2-benzofuran-1,3′-pyrrolidin]-1′-yl]carbonyl}phenyl)piperazine-1-carboxylate

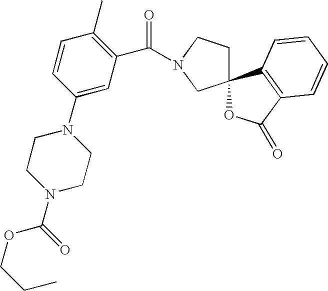

The title compound was prepared by using procedures analogous to those used for the synthesis of example 28. LC-MS: 478.2 (M+H)⁺.

### Example 96

### Prop-2-yn-1-yl 4-(4-methyl-3-{[(1R)-3-oxo-1′H,3H-spiro[2-benzofuran-1,3′-pyrrolidin]-1′-yl]carbonyl}phenyl)piperazine-1-carboxylate

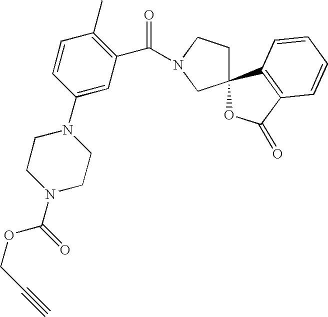

The title compound was prepared by using procedures analogous to those used for the synthesis of example 28. LC-MS: 474.2 (M+H)⁺.

### Example 97

### Isopropyl 4-(4-methyl-3-{[(1R)-3-oxo-1′H,3H-spiro[2-benzofuran-1,3′-pyrrolidin]-1′-yl]carbonyl}phenyl)piperazine-1-carboxylate

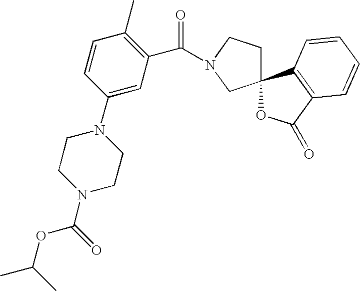

The title compound was prepared by using procedures analogous to those used for the synthesis of example 28. LC-MS: 478.2 (M+H)⁺.

### Example 98

### Isobutyl 4-(4-methyl-3-{[(1R)-3-oxo-1′H,3H-spiro[2-benzofuran-1,3′-pyrrolidin]-1′-yl]carbonyl}phenyl)piperazine-1-carboxylate

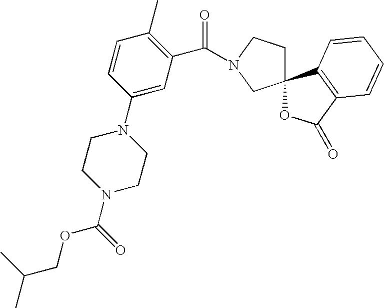

The title compound was prepared by using procedures analogous to those used for the synthesis of example 28. LC-MS: 492.3 (M+H)⁺.

### Example 99

### (1R)-1′-{2-Methyl-5-[4-(methylsulfonyl)piperazin-1-yl]benzoyl}-3H-spiro[2-benzofuran-1,3′-pyrrolidin]-3-one

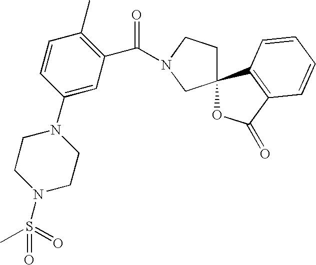

The title compound was prepared by using procedures analogous to those used for the synthesis of example 28. LC-MS: 470.2 (M+H)⁺.

### Example 100

### (1R)-1′-{5-[4-(Ethylsulfonyl)piperazin-1-yl]-2-methylbenzoyl}-3H-spiro[2-benzofuran-1,3′-pyrrolidin]-3-one

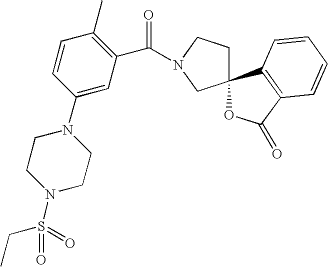

The title compound was prepared by using procedures analogous to those used for the synthesis of example 28. LC-MS: 484.2 (M+H)⁺.

### Example 101

### (1R)-1′-[5-(4-Acetylpiperazin-1-yl)-2-methylbenzoyl]-3H-spiro[2-benzofuran-1,3′-pyrrolidin]-3-one

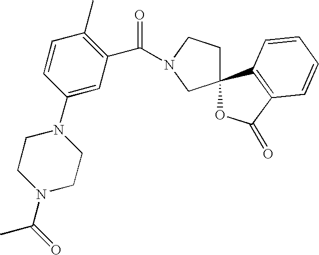

The title compound was prepared by using procedures analogous to those used for the synthesis of example 28. LC-MS: 434.2 (M+H)⁺.

### Example 102

### (1R)-1′-[2-Methyl-5-(4-propionylpiperazin-1-yl)benzoyl]-3H-spiro[2-benzofuran-1,3′-pyrrolidin]-3-one

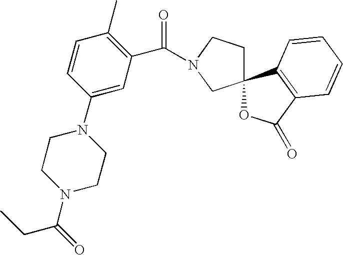

The title compound was prepared by using procedures analogous to those used for the synthesis of example 28. LC-MS: 448.2 (M+H)⁺.

### Example 103

### (1R)-1′-[5-(4-Isobutyrylpiperazin-1-yl)-2-methylbenzoyl]-3H-spiro[2-benzofuran-1,3′-pyrrolidin]-3-one

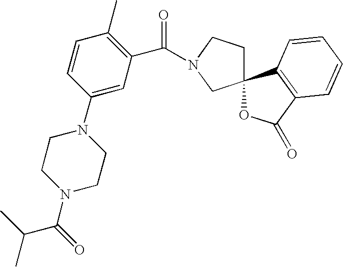

The title compound was prepared by using procedures analogous to those used for the synthesis of example 28. LC-MS: 462.3 (M+H)⁺.

### Example 104

### (1R)-1′-{5-[4-(Cyclopropylcarbonyl)piperazin-1-yl-2-methylbenzoyl]-3H-spiro[2-benzofuran-1,3′-pyrrolidin]-3-one

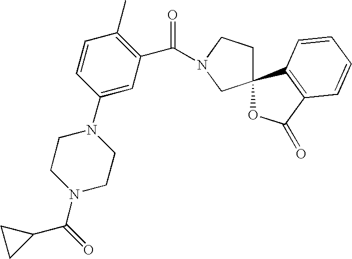

The title compound was prepared by using procedures analogous to those used for the synthesis of example 28. LC-MS: 460.3 (M+H)⁺.

### Example 105

### tert-Butyl 4-(4-methyl-3-([(1R)-3-oxo-1′H,3H-spiro[2-benzofuran-1,3′-pyrrolidin]-1′-yl]carbonyl}phenyl)-3,6-dihydropyridine-1 (2H)-carboxylate

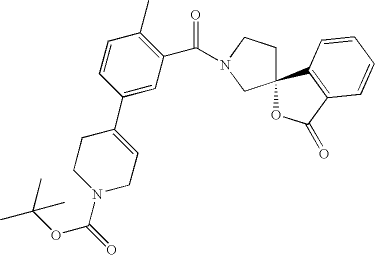

The title compound was prepared by using procedures analogous to those used for the synthesis of example 57. LC-MS: 489.3 (M+H)⁺.

### Example 106

### Methyl 4-(4-methyl-3-{[(1R)-3-oxo-1′H,3H-spiro[2-benzofuran-1,3′-pyrrolidin]-1′-yl]carbonyl}phenyl)-3,6-dihydropyridine-1(2H)-carboxylate

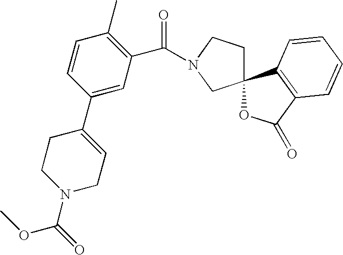

The title compound was prepared by using procedures analogous to those described for the synthesis of example 59 starting from tert-butyl 4-(4-methyl-3-{[(1R)-3-oxo-1′H,3H-spiro[2-benzofuran-1,3′-pyrrolidin]-1′-yl]carbonyl}phenyl)-3,6-dihydropyridine-1 (2H)-carboxylate (example 105). LC-MS: 447.2 (M+H)⁺.

### Example 107

### (1R)-1′-(2-Chloro-4-phenoxybenzoyl)-3H-spiro[2-benzofuran-1,3′-pyrrolidin]-3-one

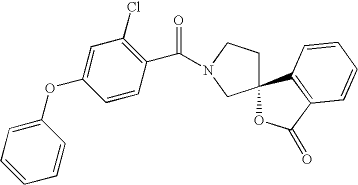

The title compound was prepared by using procedures analogous to those used for the synthesis of example 8. LC-MS: 420.1 (M+H)⁺.

### Example 108

### (1R)-1′-[2-Chloro-4-(1H-indol-6-yl)benzoyl]-3H-spiro[2-benzofuran-1,3′-pyrrolidin]-3-one

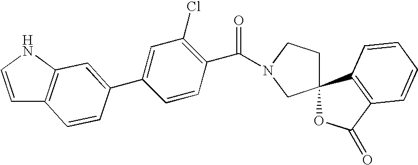

The title compound was prepared by using procedures analogous to those used for the synthesis of example 70. LC-MS: 443.1 (M+H)⁺.

### Example 109

### (1R)-1′-[4-(6-aminopyridin-2-yl)-2-chlorobenzoyl]-3H-spiro[2-benzofuran-1,3′-pyrrolidin]-3-one

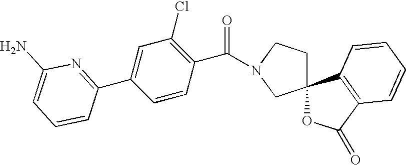

The title compound was prepared by using procedures analogous to those used for the synthesis of example 70. LC-MS: 420.0 (M+H)⁺.

### Example 110

### N-[6-(3-Chloro-4-{[(1R)-3-oxo-1′H,3H-spiro[2-benzofuran-1,3′-pyrrolidin]-1′-yl]carbonyl}phenyl)pyridin-2-yl]acetamide

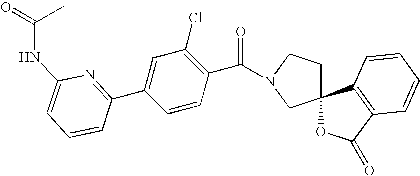

The title compound was prepared by using procedures analogous to those used for the synthesis of example 70. LC-MS: 462.1 (M+H)⁺.

### Example 111

### N-[6-(3-Chloro-4-{[(1R)-3-oxo-1′H,3H-spiro[2-benzofuran-1,3′-pyrrolidin]-1′-yl]carbonyl}phenyl)pyridin-2-yl]-2-methylpropanamide

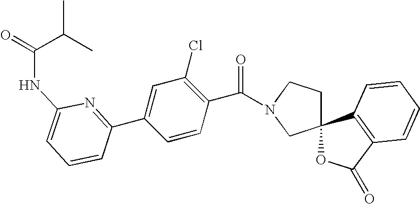

The title compound was prepared by using procedures analogous to those used for the synthesis of example 70. LC-MS: 490.1 (M+H)⁺.

### Example 112

### N-[6-(3-Chloro-4-{[(1R)-3-oxo-1′H,3H-spiro[2-benzofuran-1,3′-pyrrolidin]-1′-yl]carbonyl}phenyl)pyridin-2-yl]cyclopropanecarboxamide

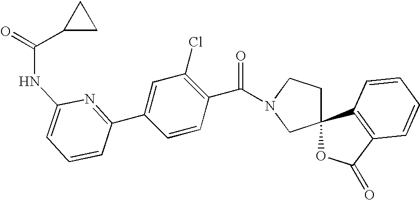

The title compound was prepared by using procedures analogous to those used for the synthesis of example 70. LC-MS: 488.1 (M+H)⁺.

### Example 113

### N-[6-(3-Chloro-4-{[(1R)-3-oxo-1′H,3H-spiro[2-benzofuran-1,3′-pyrrolidin]-1′-yl]carbonyl}phenyl)pyridin-2-yl]ethanesulfonamide

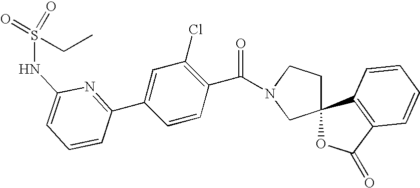

The title compound was prepared by using procedures analogous to those used for the synthesis of example 70. LC-MS: 512.1 (M+H)⁺.

### Example 114

### N-[6-(3-Chloro-4-{[(1R)-3-oxo-1′H,3H-spiro[2-benzofuran-1,3′-pyrrolidin]-1′-yl]carbonyl}phenyl)pyridin-2-yl]butanamide

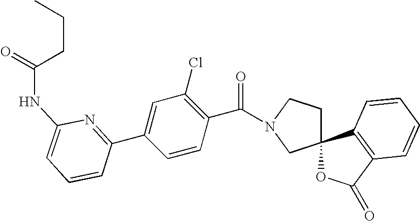

The title compound was prepared by using procedures analogous to those used for the synthesis of example 76. LC-MS: 490.1 (M+H)⁺.

### Example 115

### Methyl [6-(3-chloro-4-{[(1R)-3-oxo-1′H,3H-spiro[2-benzofuran-1,3′-pyrrolidin]-1′-yl]carbonyl}phenyl)pyridin-2-yl]carbamate

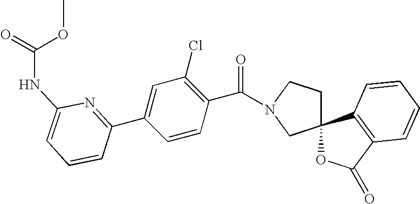

The title compound was prepared by using procedures analogous to those used for the synthesis of example 76. LC-MS: 478.1 (M+H)⁺.

### Example 116

### Ethyl [6-(3-chloro-4-{[(1R)-3-oxo-1′H,3H-spiro[2-benzofuran-1,3′-pyrrolidin]-1′-yl]carbonyl}phenyl)pyridin-2-yl]carbamate

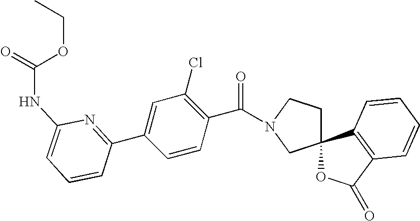

The title compound was prepared by using procedures analogous to those used for the synthesis of example 76. LC-MS: 492.1 (M+H)⁺.

### Example 117

### Propyl 16-(3-chloro-4-{[(1R)-3-oxo-1′H,3H-spiro[2-benzofuran-1,3′-pyrrolidin-1′-yl]carbonyl) phenyl)pyridin-2-yl]carbamate

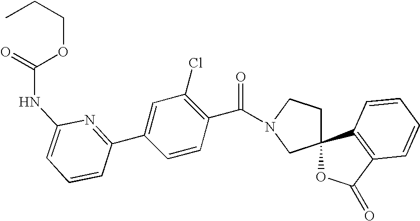

The title compound was prepared by using procedures analogous to those used for the synthesis of example 76. LC-MS: 506.1 (M+H)⁺.

### Example 118

### Isopropyl [6-(3-chloro-4-{[(1R)-3-oxo-1′H,3H-spiro[2-benzofuran-1,3′-pyrrolidin]-1′-yl]carbonyl}phenyl)pyridin-2-yl]carbamate

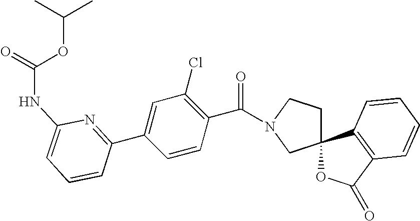

The title compound was prepared by using procedures analogous to those used for the synthesis of example 76. LC-MS: 506.1 (M+H)⁺.

### Example 119

### Isobutyl [6-(3-chloro-4-{[(1R)-3-oxo-1′H,3H-spiro[2-benzofuran-1,31-pyrrolidin]-1′-yl]carbonyl}phenyl)pyridin-2-yl]carbamate

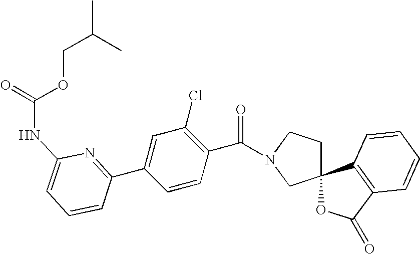

The title compound was prepared by using procedures analogous to those used for the synthesis of example 76. LC-MS: 520.1 (M+H)⁺.

### Example 120

### (1R)-1′-[2-Chloro-4-(pyridin-3-yloxy)benzoyl]-3H-spiro[2-benzofuran-1,3′-pyrrolidin]-3-one

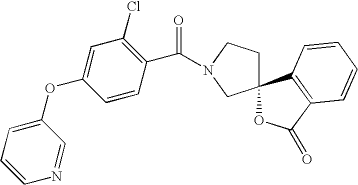

The title compound was prepared by using procedures analogous to those used for the synthesis of example 8. LC-MS: 421.1 (M+H)⁺.

### Example 121

### (1R)-1′-(2-Chloro-4-quinolin-7-ylbenzoyl)-3H-spiro[2-benzofuran-1,3′-pyrrolidin]-3-one

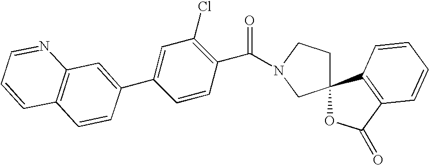

The title compound was prepared by using a palladium catalyzed coupling procedure analogous to that described for the synthesis of example 57, step 2, starting from (1R)-1′-[2-chloro-4-(4,4,5,5-tetramethyl-1,3,2-dioxaborolan-2-yl)benzoyl]-3H-spiro[2-benzofuran-1,3′-pyrrolidin]-3-one and quinolin-7-yl trifluoromethanesulfonate. LC-MS: 455.1 (M+H)⁺.

### Example 122

### 5-(3-Chloro-4-{[(1R)-3-oxo-1′H,3H-spiro[2-benzofuran-1,3′-pyrrolidin]-1′-yl]carbonyl}phenyl)-N-cyclopropylpyridine-2-carboxamide

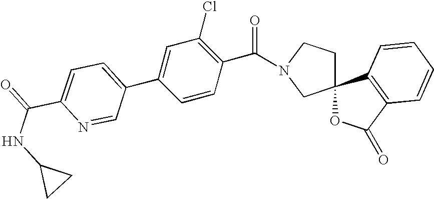

The title compound was prepared by using procedures analogous to those used for the synthesis of example 70. LC-MS: 488.2 (M+H)⁺.

### Example 123

### (1R)-1′-[4-(4-Hydroxyphenoxy)benzoyl]-3H-spiro[2-benzofuran-1,3′-pyrrolidin]-3-one

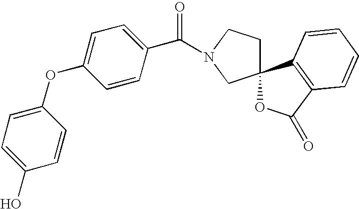

The title compound was prepared by using procedures analogous to those used for the synthesis of example 1. LC-MS: 402.2 (M+H)⁺.

### Example 124

### 5-(3-Chloro-4-{[(1R)-3-oxo-1′H,3H-spiro[2-benzofuran-1,3′-pyrrolidin]-1′-yl]carbonyl}phenyl)-N-ethylpyridine-2-carboxamide

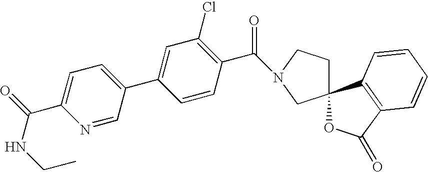

The title compound was prepared by using procedures analogous to those used for the synthesis of example 70. LC-MS: 476.2 (M+H)⁺.

### Example 125

### 5-(3-Chloro-4-{[(1R)-3-oxo-1′H,3H-spiro[2-benzofuran-1,3′-pyrrolidin]-1′-yl]carbonyl}phenyl)-N,N-diethylpyridine-2-carboxamide

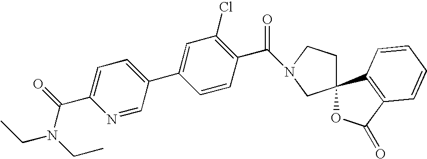

The title compound was prepared by using procedures analogous to those used for the synthesis of example 70. LC-MS: 504.2 (M+H)⁺.

### Example 126

### 5-(3-Chloro-4-{[(1R)-3-oxo-1′H,3H-spiro[2-benzofuran-1,3′-pyrrolidin]-1′-yl]carbonyl}phenyl)-N-cyclopropylpyridine-2-carboxamide

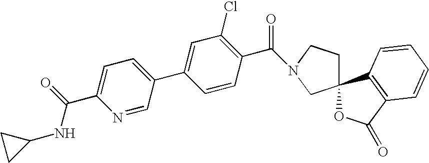

The title compound was prepared by using procedures analogous to those used for the synthesis of example 70. LC-MS: 488.2 (M+H)⁺.

### Example 127

### (1R)-1′-{4-[6-(Azetidin-1-ylcarbonyl)pyridin-3-yl]-2-chlorobenzoyl}-3H-spiro[2-benzofuran-1,3′-pyrrolidin]-3-one

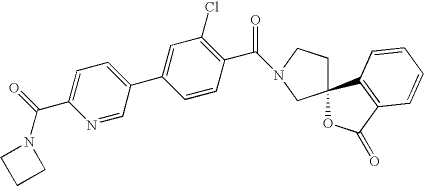

The title compound was prepared by using procedures analogous to those used for the synthesis of example 70. LC-MS: 488.1 (M+H)⁺.

### Example 128

### 5-(3-Chloro-4-{[(1R)-3-oxo-1′H,3H-spiro[2-benzofuran-1,3′-pyrrolidin]-1′-yl]carbonyl}phenyl)-N-methylpyridine-2-carboxamide

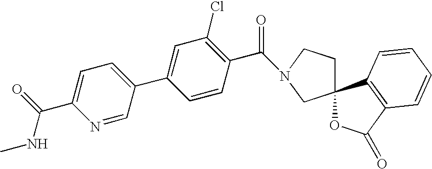

The title compound was prepared by using procedures analogous to those used for the synthesis of example 70. LC-MS: 462.1 (M+H)⁺.

### Example 129

### 5-(3-Chloro-4-{[(1R)-3-oxo-1′H,3H-spiro[2-benzofuran-1,3′-pyrrolidin]-1′-yl]carbonyl}phenyl)-N,N-dimethylpyridine-2-carboxamide

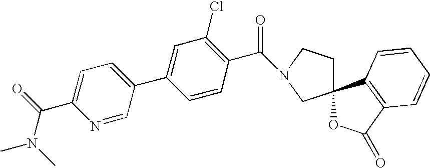

The title compound was prepared by using procedures analogous to those used for the synthesis of example 70. LC-MS: 476.2 (M+H)⁺.

### Example 130

### (1R)-1′-{2-Chloro-4-[(6-methylpyridin-3-yl)oxy]benzoyl}-3H-spiro[2-benzofuran-1,3′-pyrrolidin]-3-one

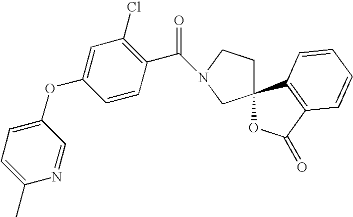

The title compound was prepared by using procedures analogous to those used for the synthesis of example 8. LC-MS: 435.1 (M+H)⁺.

### Example 131

### 6-(3-Chloro-4-{[(1R)-3-oxo-1′H,3H-spiro[2-benzofuran-1,3′-pyrrolidin]-1′-yl]carbonyl}phenyl)-N-methylpyridine-2-carboxamide

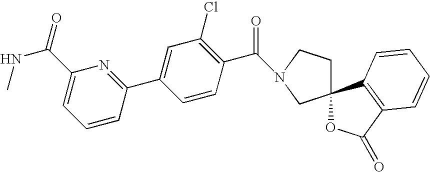

Oxalyl chloride (0.08 g, 0.0007 mol) was added to a suspension of 6-(3-chloro-4-{[(1R)-3-oxo-1′H,3H-spiro[2-benzofuran-1,3′-pyrrolidin]-1′-yl]carbonyl}phenyl) pyridine-2-carboxylic acid (0.060 g, 0.00013 mol, prepared by using procedures that were analogous to those described for the synthesis of example 70) in methylene chloride (3 mL, 0.05 mol) followed by 2 drops of DMF. The mixture was stirred at rt for 1 h. The volatiles were removed in-vacuo and the residue was azeotroped with toluene twice. The crude acyl chloride was dissolved in acetonitrile (6 mL) and divided into 6 individual reaction vessels. Each reaction vessel was treated with the corresponding amine, in this example the amine was N-methylamine (12 μL, 2.0 N in THF), and triethylamine (0.012 mL, 0.00008 mol). After stirring at rt for 30 min, the crude reaction mixture was purified by prep-LC/MS to afford the desired product. LC-MS: 462.2 (M+H)⁺.

### Example 132

### 6-(3-Chloro-4-{[(1R)-3-oxo-1′H,3H-spiro[2-benzofuran-1,3′-pyrrolidin]-1′-yl]carbonyl}phenyl)-N,N-dimethylpyridine-2-carboxamide

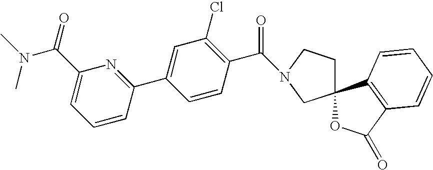

The title compound was prepared by using procedures analogous to those used for the synthesis of example 131. LC-MS: 476.1 (M+H)⁺.

### Example 133

### 6-(3-Chloro-4-{[(1R)-3-oxo-1′H,3H-spiro[2-benzofuran-1,3′-pyrrolidin]-1′-yl]carbonyl}phenyl)-N-ethylpyridine-2-carboxamide

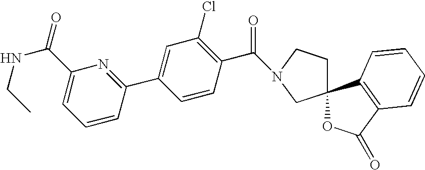

The title compound was prepared by using procedures analogous to those used for the synthesis of example 131. LC-MS: 476.1 (M+H)⁺.

### Example 134

### 6-(3-Chloro-4-{[(1R)-3-oxo-1′H,3H-spiro[2-benzofuran-1,3′-pyrrolidin]-1′-yl]carbonyl}phenyl)-N,N-diethylpyridine-2-carboxamide

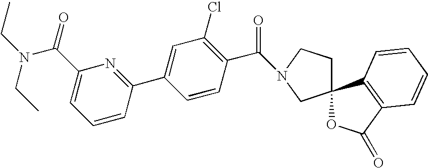

The title compound was prepared by using procedures analogous to those used for the synthesis of example 131. LC-MS: 504.1 (M+H)⁺.

### Example 135

### 6-(3-Chloro-4-([(1R)-3-oxo-1′H,3H-spiro[2-benzofuran-1,3′-pyrrolidin]-1′-yl]carbonylphenyl)-N-cyclopropylpyridine-2-carboxamide

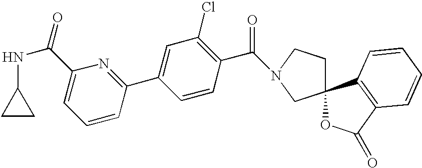

The title compound was prepared by using procedures analogous to those used for the synthesis of example 131. LC-MS: 488.1 (M+H)⁺.

### Example 136

### (1R)-1′-{4-[6-(Azetidin-1-ylcarbonyl)pyridin-2-yl]-2-chlorobenzoyl}-3H-spiro[2-benzofuran-1,3′-pyrrolidin]-3-one

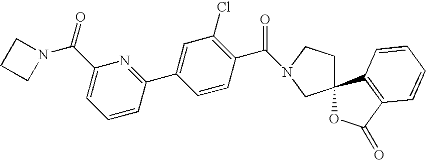

The title compound was prepared by using procedures analogous to those used for the synthesis of example 131. LC-MS: 488.1 (M+H)⁺.

### Example 137

### (1R)-1′-{2-Chloro-4-[(6-methylpyridin-2-yl)oxy]benzoyl}-3H-spiro[2-benzofuran-1,3′-pyrrolidin]-3-one

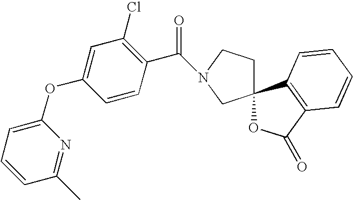

The title compound was prepared by using procedures analogous to those used for the synthesis of example 8. LC-MS: 435.1 (M+H)⁺.

### Example 138

### (1R)-1′-[4-(3-Hydroxyphenoxy)benzoyl]-3H-spiro[2-benzofuran-1,3′-pyrrolidin]-3-one

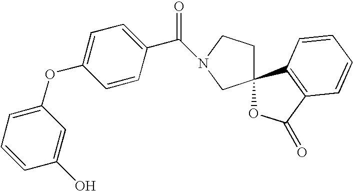

The title compound was prepared by using procedures analogous to those used for the synthesis of example 1. LC-MS: 402.2 (M+H)⁺.

### Example 139

### (1R)-1′-{2-Chloro-4-[(2-methylpyridin-3-yl)oxy]benzoyl}-3H-spiro[2-benzofuran-1,3′-pyrrolidin]-3-one

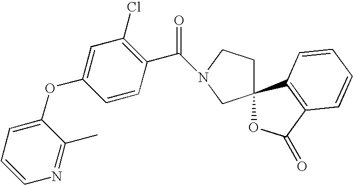

The title compound was prepared by using procedures analogous to those used for the synthesis of example 8. LC-MS: 435.2 (M+H)⁺.

### Example 140

### (1R)-1′-(2-Chloro-4-[(2,6-dimethylpyridin-4-yl)oxy]benzoyl}-3H-spiro[2-benzofuran-1,3′-pyrrolidin]-3-one

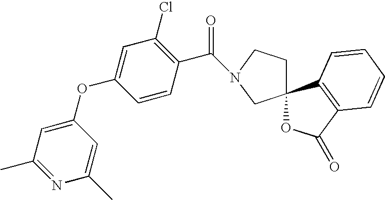

The title compound was prepared by using procedures analogous to those used for the synthesis of example 8. LC-MS: 449.2 (M+H)⁺.

### Example 141

### 6-(3-Chloro-4-{[(1R)-3-oxo-1′H,3H-spiro[2-benzofuran-1,3′-pyrrolidin]-1′-yl]carbonyl}phenoxy)-N-methylnicotinamide

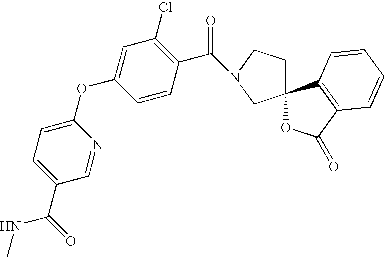

The title compound was prepared by using procedures analogous to those used for the synthesis of example 4. LC-MS: 478.0 (M+H)⁺.

### Example 142

### 6-(3-Chloro-4-{[(1R)-3-oxo-1′H,3H-spiro[2-benzofuran-1,3′-pyrrolidin]-1′-yl]carbonyl}phenoxy)-N,N-diethylnicotinamide

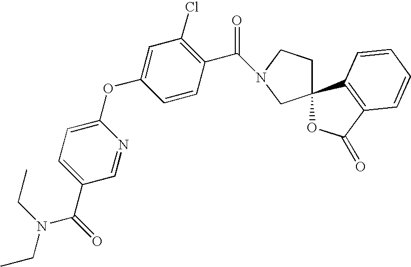

The title compound was prepared by using procedures analogous to those used for the synthesis of example 4. LC-MS: 520.1 (M+H)⁺.

### Example 143

### (1R)-1′-(4-{[3-Chloro-5-(trifluoromethyl)pyridin-2-yl]oxy}benzoyl)-3H-spiro[2-benzofuran-1,3′-pyrrolidin]-3-one

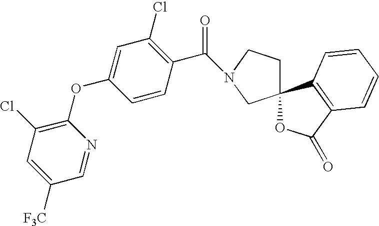

The title compound was prepared by using procedures analogous to those used for the synthesis of example 1. LC-MS: 489.1 (M+H)⁺.

### Example 144

### 5-(4-Chloro-3-{[(1R)-3-oxo-1′H,3H-spiro[2-benzofuran-1,3′-pyrrolidin]-1′-yl]carbonyl}phenyl)-N-methylpyridine-2-carboxamide

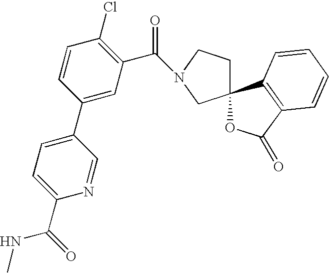

The title compound was prepared by using procedures analogous to those used for the synthesis of example 66. LC-MS: 462.1 (M+H)⁺.

### Example 145

### 5-(4-Chloro-3-{[(1R)-3-oxo-1′H,3H-spiro[2-benzofuran-1,3′-pyrrolidin]-1′-yl]carbonyl}phenyl)-N,N-dimethylpyridine-2-carboxamide

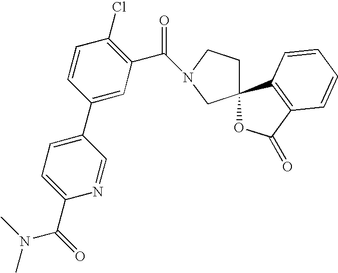

The title compound was prepared by using procedures analogous to those used for the synthesis of example 66. LC-MS: 476.1 (M+H)⁺.

### Example 146

### 5-(4-Chloro-3-{[(1R)-3-oxo-1′H,3H-spiro[2-benzofuran-1,3′-pyrrolidin]-1′-yl]carbonyl}phenyl)-N-ethylpyridine-2-carboxamide

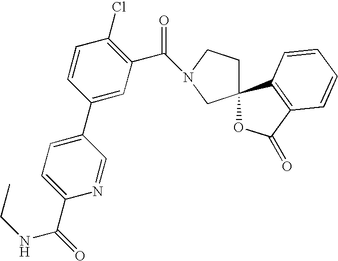

The title compound was prepared by using procedures analogous to those used for the synthesis of example 66. LC-MS: 476.1 (M+H)⁺.

### Example 147

### 5-(4-Chloro-3-{[(1R)-3-oxo-1′H,3H-spiro[2-benzofuran-1,3′-pyrrolidin]-1′-yl]carbonyl}phenyl)-N,N-diethylpyridine-2-carboxamide

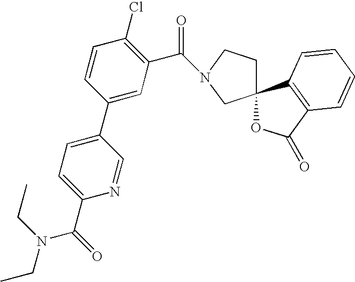

The title compound was prepared by using procedures analogous to those used for the synthesis of example 66. LC-MS: 504.2 (M+H)⁺.

### Example 148

### (1R)-1′-{4-[(6-Methylpyridazin-3-yl)oxy]benzoyl}-3H-spiro[2-benzofuran-1,3′-pyrrolidin]-3-one

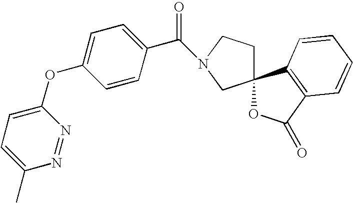

The title compound was prepared by using procedures analogous to those used for the synthesis of example 1. LC-MS: 402.2 (M+H)⁺.

### Example A

### Enzymatic Assay of 11βHSD1

All in vitro assays were performed with clarified lysates as the source of 11βHSD1 activity. HEK-293 transient transfectants expressing an epitope-tagged version of full-length human 11βHSD1 were harvested by centrifugation. Roughly 2×10⁷ cells were resuspended in 40 mL of lysis buffer (25 mM Tris-HCl, pH 7.5, 0.1 M NaCl, 1 mM MgCl₂ and 250 mM sucrose) and lysed in a microfluidizer. Lysates were clarified by centrifugation and the supernatants were aliquoted and frozen.

Inhibition of 11βHSD1 by test compounds was assessed in vitro by a Scintillation Proximity Assay (SPA). Dry test compounds were dissolved at 5 mM in DMSO. These were diluted in DMSO to suitable concentrations for the SPA assay. 0.8 μL of 2-fold serial dilutions of compounds were dotted on 384 well plates in DMSO such that 3 logs of compound concentration were covered. 20 μL of clarified lysate was added to each well. Reactions were initiated by addition of 20 μL of substrate-cofactor mix in assay buffer (25 mM Tris-HCl, pH 7.5, 0.1 M NaCl, 1 mM MgCl₂) to final concentrations of 400 μM NADPH, 25 nM ³H-cortisone and 0.007% Triton X-100. Plates were incubated at 37° C. for one hour. Reactions were quenched by addition of 40 μL of anti-mouse coated SPA beads that had been pre-incubated with 10 μM carbenoxolone and a cortisol-specific monoclonal antibody. Quenched plates were incubated for a minimum of 30 minutes at RT prior to reading on a Topcount scintillation counter. Controls with no lysate, inhibited lysate, and with no mAb were run routinely. Roughly 30% of input cortisone is reduced by 11βHSD1 in the uninhibited reaction under these conditions.

Test compounds having an IC₅₀ value less than about 20 μM according to this assay were considered active.

### Example B

### Cell-Based Assays for HSD Activity

Peripheral blood mononuclear cells (PBMCs) were isolated from normal human volunteers by Ficoll density centrifugation. Cells were plated at 4×10⁵ cells/well in 200 μL of AIM V (Gibco-BRL) media in 96 well plates. The cells were stimulated overnight with 50 ng/ml recombinant human IL-4 (R&D Systems). The following morning, 200 nM cortisone (Sigma) was added in the presence or absence of various concentrations of compound. The cells were incubated for 48 hours and then supernatants were harvested. Conversion of cortisone to cortisol was determined by a commercially available ELISA (Assay Design).

Test compounds having an IC₅₀ value less than about 20 μM according to this assay were considered active.

### Example C

### Cellular Assay to Evaluate MR Antagonism

Assays for MR antagonism can be performed essentially as described (Jausons-Loffreda et al. J Biolumin and Chemilumin, 1994, 9: 217-221). Briefly, HEK293/MSR cells (Invitrogen Corp.) are co-transfected with three plasmids: 1) one designed to express a fusion protein of the GAL4 DNA binding domain and the mineralocorticoid receptor ligand binding domain, 2) one containing the GAL4 upstream activation sequence positioned upstream of a firefly luciferase reporter gene (pFR-LUC, Stratagene, Inc.), and 3) one containing the Renilla luciferase reporter gene cloned downstream of a thymidine kinase promoter (Promega). Transfections were performed using the FuGENE6 reagent (Roche). Transfected cells can be ready for use in subsequent assays 24 hours post-transfection.

In order to evaluate a compound's ability to antagonize the MR, test compounds are diluted in cell culture medium (E-MEM, 10% charcoal-stripped FBS, 2 mM L-glutamine) supplemented with 1 nM aldosterone and applied to the transfected cells for 16-18 hours. After the incubation of the cells with the test compound and aldosterone, the activity of firefly luciferase (indicative of MR agonism by aldosterone) and Renilla luciferase (normalization control) are determined using the Dual-Glo Luciferae Assay System (Promega). Antagonism of the mineralocorticoid receptor is determined by monitoring the ability of a test compound to attenuate the aldosterone-induced firefly luciferase activity.

Compounds having an IC₅₀ of 100 μM or less are considered active.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A compound of Formula Ia or Ib: 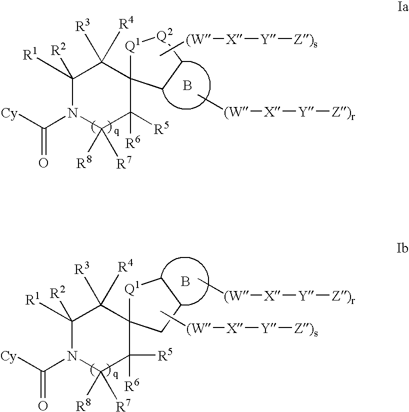
or pharmaceutically acceptable salt or prodrug thereof, wherein:
Cy is aryl, heteroaryl, cycloalkyl, or heterocycloalkyl, each optionally substituted by 1, 2, 3, 4 or 5 —U-T-W—X—Y-Z;
Q¹ is O, S, NH, CH₂, CO, CS, SO, SO₂, OCH₂, SCH₂, NHCH₂, CH₂CH₂, COCH₂, CONH, COO, SOCH₂, SONH, SO₂CH₂, or SO₂NH;
Q² is O, S, NH, CH₂, CO, CS, SO, SO₂, OCH₂, SCH₂, NHCH₂, CH₂CH₂, COCH₂, CONH, COO, SOCH₂, SONH, SO₂CH₂, or SO₂NH;
ring B is an aryl, heteroaryl, cycloalkyl, or heterocycloalkyl group fused with the ring containing Q¹ and Q²;
R¹, R², R³, R⁴, R⁵, R⁶, R⁷, and R⁸ are each, independently, H or —W′—X′—Y′-Z′;
or R¹ and R² together with the C atom to which they are attached form a 3-20 membered cycloalkyl group or a 3-20 membered heterocycloalkyl group optionally substituted by 1 or 2 —W″—X″—Y″-Z″;
or R³ and R⁴ together with the C atom to which they are attached form a 3-20 membered cycloalkyl group or a 3-20 membered heterocycloalkyl group optionally substituted by 1 or 2 —W″—X″—Y″-Z″;
or R⁵ and R⁶ together with the C atom to which they are attached form a 3-20 membered cycloalkyl group or a 3-20 membered heterocycloalkyl group optionally substituted by 1 or 2 —W″—X″—Y″-Z″;
or R⁷ and R⁸ together with the C atom to which they are attached form a 3-20 membered cycloalkyl group or a 3-20 membered heterocycloalkyl group optionally substituted by 1 or 2 —W″—X″—Y″-Z″;
or R¹ and R⁵ together form an C₁₋₄ alkylene bridge optionally substituted by 1 or 2 —W″—X″—Y″-Z″;
or R³ and R⁵ together form an C₁₋₄ alkylene bridge optionally substituted by 1 or 2 —W″—X″—Y″-Z″;
U is absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;
T is absent, C₁₋₆ alkylenyl, C₂₋₆ alkenylenyl, C₂₋₆ alkynylenyl, aryl, aryloxy, cycloalkyl, heteroaryl, heteroaryloxy, or heterocycloalkyl, wherein said C₁₋₆ alkylenyl, C₂₋₆ alkenylenyl, C₂₋₆ alkynylenyl, cycloalkyl, heteroaryl or heterocycloalkyl is optionally substituted by one or more halo, CN, NO₂, OH, C₁₋₄ alkoxy, C₁₋₄ haloalkoxy, amino, C₁₋₄ alkylamino or C₂₋₈ dialkylamino;
W, W′ and W″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;
X, X′ and X″ are each, independently, absent, C₁₋₆ alkylenyl, C₂₋₆ alkenylenyl, C₂₋₆ alkynylenyl, aryl, cycloalkyl, heteroaryl or heterocycloalkyl, wherein said C₁₋₆ alkylenyl, C₂₋₆ alkenylenyl, C₂₋₆ alkynylenyl, cycloalkyl, heteroaryl or heterocycloalkyl is optionally substituted by one or more halo, CN, NO₂, OH, C₁₋₄ alkoxy, C₁₋₄ haloalkoxy, amino, C₁₋₄ alkylamino or C₂₋₈ dialkylamino;
Y, Y′ and Y″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;
Z, Z′ and Z″ are each, independently, H, halo, CN, NO₂, OH, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, aryl, cycloalkyl, heteroaryl or heterocycloalkyl, wherein said C₁₋₆ alkyl, C₂₋₆ alkenyl, C₂₋₆ alkynyl, aryl, cycloalkyl, heteroaryl or heterocycloalkyl is optionally substituted by 1, 2 or 3 halo, C₁₋₆ alkyl, C₁₋₆ hydroxyalkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, —C₁₋₄ alkyl-OC(O)NR^{c}R^{d}, NR^{c}R^{d}, NR^{c}C(O)R^{d}, NR^{c}C(O)OR, S(O)R^{b}, S(O)NR^{c}R^{d}, S(O)₂R^{b}, NR^{c}S(O)₂R^{b} or S(O)₂NR^{c}R^{d};
wherein two —W—X—Y-Z together with the atom to which they are both attached optionally form a 3-20 membered cycloalkyl group or 3-20 membered heterocycloalkyl group optionally substituted by 1, 2 or 3 —W″—X″—Y″-Z″;
wherein two —W′—X′—Y′-Z′ together with the atom to which they are both attached optionally form a 3-20 membered cycloalkyl group or 3-20 membered heterocycloalkyl group optionally substituted by 1, 2 or 3 —W″—X″—Y″-Z″;
wherein —W—X—Y-Z is other than H;
wherein —W′—X′—Y′-Z′ is other than H;
wherein —W″—X″—Y″-Z″ is other than H;
R^{a} is H, C₁₋₆ alkyl, C₁₋₆ haloalkyl, C₂₋₆ alkenyl, C₂₋₆ alkynyl, aryl, cycloalkyl, heteroaryl or heterocycloalkyl;
R^{b} is H, C₁₋₆ alkyl, C₁₋₆ haloalkyl, C₂₋₆ alkenyl, C₂₋₆ alkynyl, aryl, cycloalkyl, heteroaryl or heterocycloalkyl;
R^{c} is H, C₁₋₆ alkyl, C₂₋₆ haloalkyl, C₂₋₆ alkenyl, C₂₋₆ alkynyl, aryl, cycloalkyl, arylalkyl, or cycloalkylalkyl;
or R^{c} and R^{d} together with the N atom to which they are attached form a 4-, 5-, 6- or 7-membered heterocycloalkyl group;
R^{e} and R^{f} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
or R^{e} and R^{f} together with the N atom to which they are attached form a 4-, 5-, 6- or 7-membered heterocycloalkyl group;
q is 0, 1, or 2;
r is 0, 1 or 2; and
s is 0, 1 or 2;
with the provisos:
a) when the compound has Formula Ia, Q¹ is CO, and Q² is NH, then s is 0;
b) when the compound has Formula Ia, Q¹ is CH₂, Q₂ is CH₂, and q is 1, then r is 1 or 2;
c) when the compound has Formula Ib, Q¹ is NH, and Q² is CONH, then s is 0;
d) when the compound has Formula Ib, Q¹ is CO, Q² is NH, then r is 1 or 2; and
e) Cy is other than cyclopropyl substituted by 1 or 2 —U-T-W—X—Y-Z.

2. The compound of claim 1 having Formula Ia.

3. The compound of claim 1 having Formula Ib.

4. The compound of claim 1 wherein Cy is aryl or heteroaryl substituted by 1, 2, 3, 4 or 5 —U-T-W—X—Y-Z.

5. The compound of claim 1 wherein Cy is phenyl substituted by 1, 2, 3, 4 or 5 —U-T-W—X—Y-Z.

6. The compound of claim 1 having Formula Ia wherein Q¹ and Q² are each, independently, O, S, NH, CH₂, CO, CS, SO, or SO₂, wherein each of said NH and CH₂ is optionally substituted by —W″—X″—Y″-Z″.

7. The compound of claim 1 having Formula Ia wherein Q¹ is O, NH, CO or CH₂ and Q² is CO, CH₂, NH, NHCH₂, or SO₂, wherein each of said NH, NHCH₂, and CH₂ is optionally substituted by —W″—X″—Y″-Z″.

8. The compound of claim 1 having Formula Ia wherein Q¹ is O and Q² is CO.

9. The compound of claim 1 wherein ring B is phenyl or pyridyl.

10. The compound of claim 1 wherein R¹, R², R³, R⁴, R⁵, R⁶, R⁷, and R⁸ are each H.

11. The compound of claim 1 wherein q is 0.

12. The compound of claim 1 wherein q is 1.

13. The compound of claim 1 wherein s is 0.

14. The compound of claim 1 wherein r is 0.

15. The compound of claim 1 wherein —U-T-W—X—Y-Z is halo, cyano, C₁₋₄ cyanoalkyl, nitro, C₁₋₄ nitroalkyl, C₁₋₄ alkyl, C₁₋₄ haloalkyl, C₁₋₄ alkoxy, C₁₋₄ haloalkoxy, OH, C₁₋₈ alkoxyalkyl, amino, C₁₋₄ alkylamino, C₂₋₈ dialkylamino, aryl, heteroaryl, cycloalkyl, heterocycloalkyl, arylalkyl, heteroarylalkyl, cycloalkylalkyl, or heterocycloalkylalkyl.

16. The compound of claim 1 wherein U and T are absent.

17. The compound of claim 1 wherein:
—U-T-W—X—Y-Z is halo, C₁₋₆ alkyl, amino, OH, OC(O)R^{b}, Z, —O-Z, —O—(C₁₋₄ alkyl)-Z, or —NHC(O)-Z; and
Z is aryl, cycloalkyl, heteroaryl or heterocycloalkyl, each optionally substituted by 1, 2 or 3 halo, C₁₋₆ alkyl, C₁₋₆ hydroxyalkyl, heterocycloalkyl, CN, OR^{a}, C(O)R^{b}, C(O)NR^{c}R^{d}, C(O)OR^{a}, —C₁₋₄ alkyl-OC(O)NR^{c}R^{d}, NR^{c}R^{d}, NR^{c}C(O)R^{d}, NR^{c}C(O)OR^{a}, S(O)₂R^{b}, or NR^{c}S(O)₂R^{b}.

18. The compound of claim 1 wherein —W′—X′—Y′-Z′ is halo, cyano, C₁₋₄ cyanoalkyl, nitro, C₁₋₄ nitroalkyl, C₁₋₄ alkyl, C₁₋₄ haloalkyl, C₁₋₄ alkoxy, C₁₋₄ haloalkoxy, OH, C₁₋₈ alkoxyalkyl, amino, C₁₋₄ alkylamino, C₂₋₈ dialkylamino, aryl, heteroaryl, cycloalkyl, heterocycloalkyl, arylalkyl, heteroarylalkyl, cycloalkylalkyl, or heterocycloalkylalkyl.

19. The compound of claim 1 having Formula II: 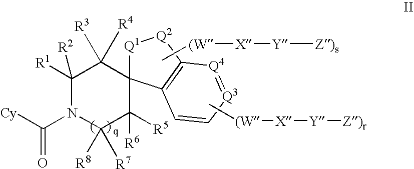
wherein:
Q³ and Q⁴ are each, independently, CH or N;
r is 0, 1 or 2; and
s is 0, 1 or 2.

20. The compound of claim 19 wherein Q¹ is O, NH, CH₂ or CO, wherein each of said NH and CH₂ is optionally substituted by —W″—X″—Y″-Z″.

21. The compound of claim 19 wherein Q² is O, S, NH, CH₂, CO, or SO₂, wherein each of said NH and CH₂ is optionally substituted by —W″—X″—Y″-Z″.

22. The compound of claim 19 wherein one of Q¹ and Q² is CO and the other is O, NH, or CH₂, wherein each of said NH and CH₂ is optionally substituted by —W″—X″—Y″-Z″.

23. The compound of claim 19 wherein one of Q¹ and Q² is CH₂ and the other is O, S, NH, or CH₂, wherein each of said NH and CH₂ is optionally substituted by —W″—X″—Y″-Z″.

24. The compound of claim 19 wherein one of Q¹ and Q² is O and the other is CO or CONH, wherein said CONH is optionally substituted by —W″—X″—Y″-Z″.

25. The compound of claim 19 wherein Q³ is CH optionally substituted by —W″—X″—Y″-Z″.

26. The compound of claim 19 wherein Q³ is N.

27. The compound of claim 19 wherein Q⁴ is CH optionally substituted by —W″—X″—Y″-Z″.

28. The compound of claim 19 wherein Q⁴ is N.

29. The compound of claim 19 wherein r is 0 or 1.

30. The compound of claim 19 wherein s is 0 or 1.

31. The compound of claim 1 having Formula III: 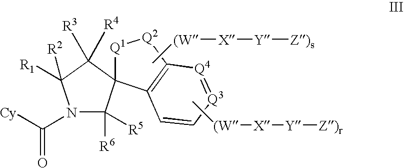
wherein:
Q³ and Q⁴ are each, independently, CH or N;
r is 0, 1 or 2; and
s is 0, 1 or 2.

32. The compound of claim 31 wherein Q¹ is O, NH, CH₂ or CO, wherein each of said NH and CH₂ is optionally substituted by —W″—X″—Y″-Z″.

33. The compound of claim 31 wherein Q² is O, S, NH, CH₂, CO, or SO₂, wherein each of said NH and CH₂ is optionally substituted by —W″—X″—Y″-Z″.

34. The compound of claim 31 wherein one of Q¹ and Q² is CO and the other is O, NH, or CH₂, wherein each of said NH and CH₂ is optionally substituted by —W″—X″—Y″-Z″.

35. The compound of claim 31 wherein one of Q¹ and Q² is CH₂ and the other is O, S, NH, or CH₂, wherein each of said NH and CH₂ is optionally substituted by —W″—X″—Y″-Z″.

36. The compound of claim 31 wherein one of Q¹ and Q² is O and the other is CO or CONH, wherein said CONH is optionally substituted by —W″—X″—Y″-Z″.

37. The compound of claim 31 wherein Q³ is CH optionally substituted by —W″—X″—Y″-Z″.

38. The compound of claim 31 wherein Q³ is N.

39. The compound of claim 31 wherein Q⁴ is CH optionally substituted by —W″—X″—Y″-Z″.

40. The compound of claim 31 wherein Q⁴ is N.

41. The compound of claim 31 wherein r is 0 or 1.

42. The compound of claim 31 wherein s is 0 or 1.

43. A compound of claim 1 selected from:
(1R)-1′-(4-Phenoxybenzoyl)-3H-spiro[2-benzofuran-1,3′-pyrrolidin]-3-one; 1′-(3-Phenoxybenzoyl)-3H-spiro[2-benzofuran-1,3′-pyrrolidin]-3-one;
(1R)-1′-(3-Bromobenzoyl)-3H-spiro[2-benzofuran-1,3′-pyrrolidin]-3-one;
(1R)-1′-[4-(Benzyloxy)benzoyl]-3H-spiro[2-benzofuran-1,3′-pyrrolidin]-3-one;
(1R)-1′-[4-(Cyclohexyloxy)benzoyl]-3H-spiro[2-benzofuran-1,3′-pyrrolidin]-3-one;
(1R)-1′-[4-(Pyridin-2-yloxy)benzoyl]-3H-spiro[2-benzofuran-1,3′-pyrrolidin]-3-one;
(1R)-1′-[4-(Pyrazin-2-yloxy)benzoyl]-3H-spiro[2-benzofuran-1,3′-pyrrolidin]-3-one;
(1R)-1′-[3-(2-Chlorophenoxy)benzoyl]-3H-spiro[2-benzofuran-1,3′-pyrrolidin]-3-one;
(1R)-1′-[3-(3-Chlorophenoxy)benzoyl]-3H-spiro[2-benzofuran-1,3′-pyrrolidin]-3-one;
(1R)-1′-[3-(4-Chlorophenoxy)benzoyl]-3H-spiro[2-benzofuran-1,3′-pyrrolidin]-3-one;
(1R)-1′-(Biphenyl-4-ylcarbonyl)-3H-spiro[2-benzofuran-1,3′-pyrrolidin]-3-one;
(1R)-1′-[2-Fluoro-4-(pyrazin-2-yloxy)benzoyl]-3H-spiro[2-benzofuran-1,3′-pyrrolidin]-3-one;
(1R)-1′-[2-Chloro-4-(pyrazin-2-yloxy)benzoyl]-3H-spiro[2-benzofuran-1,3′-pyrrolidin]-3-one;
(1R)-1′-{2-Chloro-4-[(3-chloropyrazin-2-yl)oxy]benzoyl}-3H-spiro[2-benzofuran-1,3′-pyrrolidin]-3-one;
(1R)-1′-{2-Chloro-4-[(3,6-dimethylpyrazin-2-yl)oxy]benzoyl}-3H-spiro[2-benzofuran-1,3′-pyrrolidin]-3-one;
(1R)-1′-[2-Chloro-4-(quinoxalin-2-yloxy)benzoyl]-3H-spiro[2-benzofuran-1,3′-pyrrolidin]-3-one;
(1R)-1′-[2-Chloro-4-(pyrimidin-2-yloxy)benzoyl]-3H-spiro[2-benzofuran-1,3′-pyrrolidin]-3-one;
(1R)-1′-{4-[(4-Amino-5-fluoropyrimidin-2-yl)oxy]-2-chlorobenzoyl}-3H-spiro[2-benzofuran-1,3′-pyrrolidin]-3-one;
(1R)-1′-{2-Chloro-4-[(4-chloropyrimidin-2-yl)oxy]benzoyl}-3H-spiro[2-benzofuran-1,3′-pyrrolidin]-3-one;
(1R)-1′-{2-Chloro-4-[(6-chloro-9H-purin-2-yl)oxy]benzoyl}-3H-spiro[2-benzofuran-1,3′-pyrrolidin]-3-one;
(1R)-1′-{2-Chloro-4-[(6-chloropyrazin-2-yl)oxy]benzoyl}-3H-spiro[2-benzofuran-1,3′-pyrrolidin]-3-one;
(1R)-1′-(4-Bromo-2-chlorobenzoyl)-3H-spiro[2-benzofuran-1,3′-pyrrolidin]-3-one;
(1R)-1′-[2-Chloro-5-(pyrazin-2-yloxy)benzoyl]-3H-spiro[2-benzofuran-1,3′-pyrrolidin]-3-one;
(1R)-1′-(4-Aminobenzoyl)-3H-spiro[2-benzofuran-1,3′-pyrrolidin]-3-one;
4-Fluoro-N-{4-[(3-oxo-1′H,3H-spiro[2-benzofuran-1,3′-pyrrolidin]-1′-yl)carbonyl]phenyl}benzamide;
tert-Butyl 4-(3-chloro-4-{[(1R)-3-oxo-1′H,3H-spiro[2-benzofuran-1,3′-pyrrolidin]-1′-yl]carbonyl}phenyl)piperazine-1-carboxylate;
(1R)-1′-(2-Chloro-4-piperazin-1-ylbenzoyl)-3H-spiro[2-benzofuran-1,3′-pyrrolidin]-3-one dihydrochloride;
(1R)-1′-[4-(4-Acetylpiperazin-1-yl)-2-chlorobenzoyl]-3H-spiro[2-benzofuran-1,3′-pyrrolidin]-3-one;
(1R)-1′-[2-Chloro-4-(4-propionylpiperazin-1-yl)benzoyl]-3H-spiro[2-benzofuran-1,3′-pyrrolidin]-3-one;
(1R)-1′-[4-(4-Butyrylpiperazin-1-yl)-2-chlorobenzoyl]-3H-spiro[2-benzofuran-1,3′-pyrrolidin]-3-one;
(1R)-1′-{2-Chloro-4-[4-(cyclopropylcarbonyl)piperazin-1-yl]benzoyl}-3H-spiro[2-benzofuran-1,3′-pyrrolidin]-3-one;
Methyl 4-(3-chloro-4-{[(1R)-3-oxo-1′H,3H-spiro[2-benzofuran-1,3′-pyrrolidin]-1′-yl]carbonyl}phenyl)piperazine-1-carboxylate;
Ethyl 4-(3-chloro-4-{[(1R)-3-oxo-1′H,3H-spiro[2-benzofuran-1,3′-pyrrolidin]-1′-yl]carbonyl}phenyl)piperazine-1-carboxylate;
Propyl 4-(3-chloro-4-{[(1R)-3-oxo-1′H,3H-spiro[2-benzofuran-1,3′-pyrrolidin]-1′-yl]carbonyl}phenyl)piperazine-1-carboxylate;
Isobutyl 4-(3-chloro-4-{[(1R)-3-oxo-1′H,3H-spiro[2-benzofuran-1,3′-pyrrolidin]-1′-yl]carbonyl}phenyl)piperazine-1-carboxylate;
(1R)-1′-{2-Chloro-4-[4-(ethylsulfonyl)piperazin-1-yl]benzoyl}-3H-spiro[2-benzofuran-1,3′-pyrrolidin]-3-one;
tert-Butyl 4-(3-methyl-4-{[(1R)-3-oxo-1′H,3H-spiro[2-benzofuran-1,3′-pyrrolidin]-1′-yl]carbonyl}phenyl)piperazine-1-carboxylate;
(1R)-1′-(2-Methyl-4-piperazin-1-ylbenzoyl)-3H-spiro[2-benzofuran-1,3′-pyrrolidin]-3-one dihydrochloride;
Methyl 4-(3-methyl-4-{[(1R)-3-oxo-1′H,3H-spiro[2-benzofuran-1,3′-pyrrolidin]-1′-yl]carbonyl}phenyl)piperazine-1-carboxylate;
Ethyl 4-(3-methyl-4-{[(1R)-3-oxo-1′H,3H-spiro[2-benzofuran-1,3′-pyrrolidin]-1′-yl]carbonyl}phenyl)piperazine-1-carboxylate;
Propyl 4-(3-methyl-4-{[(1R)-3-oxo-1′H,3H-spiro[2-benzofuran-1,3′-pyrrolidin]-1′-yl]carbonyl}phenyl)piperazine-1-carboxylate;
Prop-2-yn-1-yl 4-(3-methyl-4-{[(1R)-3-oxo-1′H,3H-spiro[2-benzofuran-1,3′-pyrrolidin]-1′-yl]carbonyl}phenyl)piperazine-1-carboxylate;
Isopropyl 4-(3-methyl-4-{[(1R)-3-oxo-1′H,3H-spiro[2-benzofuran-1,3′-pyrrolidin]-1′-yl]carbonyl}phenyl)piperazine-1-carboxylate;
Isobutyl 4-(3-methyl-4-{[(1R)-3-oxo-1′H,3H-spiro[2-benzofuran-1,3′-pyrrolidin]-1′-yl]carbonyl}phenyl)piperazine-1-carboxylate;
(1R)-1′-{2-Methyl-4-[4-(methylsulfonyl)piperazin-1-yl]benzoyl}-3H-spiro[2-benzofuran-1,3′-pyrrolidin]-3-one;
(1R)-1′-{4-[4-(Ethylsulfonyl)piperazin-1-yl]-2-methylbenzoyl}-3H-spiro[2-benzofuran-1,3′-pyrrolidin]-3-one;
(1R)-1′-[4-(4-Acetylpiperazin-1-yl)-2-methylbenzoyl]-3H-spiro[2-benzofuran-1,3′-pyrrolidin]-3-one;
(1R)-1′-[2-Methyl-4-(4-propionylpiperazin-1-yl)benzoyl]-3H-spiro[2-benzofuran-1,3′-pyrrolidin]-3-one;
(1R)-1′-[4-(4-Isobutyrylpiperazin-1-yl)-2-methylbenzoyl]-3H-spiro[2-benzofuran-1,3′-pyrrolidin]-3-one;
(1R)-1′-{4-[4-(Cyclopropylcarbonyl)piperazin-1-yl]-2-methylbenzoyl}-3H-spiro[2-benzofuran-1,3′-pyrrolidin]-3-one;
(1R)-1′-[2-Chloro-4-(9H-purin-9-yl)benzoyl]-3H-spiro[2-benzofuran-1,3′-pyrrolidin]-3-one;
(1R)-1′-[4-(2-Oxopyrrolidin-1-yl)benzoyl]-3H-spiro[2-benzofuran-1,3′-pyrrolidin]-3-one;
(1R)-1′-[4-(2-Oxo-1,3-oxazolidin-3-yl)benzoyl]-3H-spiro[2-benzofuran-1,3′-pyrrolidin]-3-one;
(1R)-1′-[2-Chloro-4-(3-methyl-1H-pyrazol-1-yl)benzoyl]-3H-spiro[2-benzofuran-1,3′-pyrrolidin]-3-one;
(1R)-1′-[2-Chloro-4-(1H-pyrazol-1-yl)benzoyl]-3H-spiro[2-benzofuran-1,3′-pyrrolidin]-3-one;
(1R)-1′-(4-Morpholin-4-ylbenzoyl)-3H-spiro[2-benzofuran-1,3′-pyrrolidin]-3-one;
tert-Butyl 4-(3-chloro-4-{[(1R)-3-oxo-1′H,3H-spiro[2-benzofuran-1,3′-pyrrolidin]-1′-yl]carbonyl}phenyl)-3,6-dihydropyridine-1(2H)-carboxylate;
(1R)-1′-[2-Chloro-4-(1,2,3,6-tetrahydropyridin-4-yl)benzoyl]-3H-spiro[2-benzofuran-1,3′-pyrrolidin]-3-one;
Methyl 4-(3-chloro-4-{[(1R)-3-oxo-1′H,3H-spiro[2-benzofuran-1,3′-pyrrolidin]-1′-yl]carbonyl}phenyl)-3,6-dihydropyridine-1(2H)-carboxylate;
(1R)-1′-[2-Chloro-4-(1-isobutyryl-1,2,3,6-tetrahydropyridin-4-yl)benzoyl]-3H-spiro[2-benzofuran-1,3′-pyrrolidin]-3-one;
(1R)-1′-[2-Chloro-4-(1-isobutyrylpiperidin-4-yl)benzoyl]-3H-spiro[2-benzofuran-1,3′-pyrrolidin]-3-one;
Methyl 4-(4-{[(1R)-3-oxo-1′H,3H-spiro[2-benzofuran-1,3′-pyrrolidin]-1′-yl]carbonyl}phenyl)piperidine-1-carboxylate;
(1R)-1′-(5-Bromo-2-chlorobenzoyl)-3H-spiro[2-benzofuran-1,3′-pyrrolidin]-3-one;
(1R)-1′-(2-Chloro-4-hydroxybenzoyl)-3H-spiro[2-benzofuran-1,3′-pyrrolidin]-3-one;
(1R)-1′-(2-Chloro-5-hydroxybenzoyl)-3H-spiro[2-benzofuran-1,3′-pyrrolidin]-3-one;
(1R)-1′-[2-Chloro-4-(5-methoxypyridin-3-yl)benzoyl]-3H-spiro[2-benzofuran-1,3′-pyrrolidin]-3-one;
(1R)-1′-[2-Chloro-4-(3,5-dimethylisoxazol-4-yl)benzoyl]-3H-spiro[2-benzofuran-1,3′-pyrrolidin]-3-one;
(1R)-1′-[2-Chloro-4-(6-methoxypyridin-3-yl)benzoyl]-3H-spiro[2-benzofuran-1,3′-pyrrolidin]-3-one;
(1R)-1′-(2-Chloro-4-pyrimidin-5-ylbenzoyl)-3H-spiro[2-benzofuran-1,3′-pyrrolidin]-3-one;
(1R)-1′-(2-Chloro-4-pyrazin-2-ylbenzoyl)-3H-spiro[2-benzofuran-1,3′-pyrrolidin]-3-one;
3′-Chloro-4′-{[(1R)-3-oxo-1′H,3H-spiro[2-benzofuran-1,3′-pyrrolidin]-1′-yl]carbonyl}biphenyl-3-carbonitrile;
(1R)-1′-[4-(1,3-Benzodioxol-5-yl)-2-chlorobenzoyl]-3H-spiro[2-benzofuran-1,3′-pyrrolidin]-3-one;
(1R)-1′-{[3-Chloro-3′-(hydroxymethyl)biphenyl-4-yl]carbonyl}-3H-spiro[2-benzofuran-1,3′-pyrrolidin]-3-one;
3′-Chloro-4′-{[(1R)-3-oxo-1′H,3H-spiro[2-benzofuran-1,3′-pyrrolidin]-1′-yl]carbonyl}biphenyl-3-carboxamide;
(1R)-1′-[(3′-Amino-3-chlorobiphenyl-4-yl)carbonyl]-3H-spiro[2-benzofuran-1,3′-pyrrolidin]-3-one;
Methyl (3′-chloro-4′-{[(1R)-3-oxo-1′H,3H-spiro[2-benzofuran-1,3′-pyrrolidin]-1′-yl]carbonyl}biphenyl-3-yl)carbamate;
Propyl (3′-chloro-4′-{[(1R)-3-oxo-1′H,3H-spiro[2-benzofuran-1,3′-pyrrolidin]-1′-yl]carbonyl}biphenyl-3-yl)carbamate;
Isobutyl (3′-chloro-4′-{[(1R)-3-oxo-1′H,3H-spiro[2-benzofuran-1,3′-pyrrolidin]-1′-yl]carbonyl}biphenyl-3-yl)carbamate;
(1R)-1′-{[3-Chloro-3′-(2-oxopyrrolidin-1-yl)biphenyl-4-yl]carbonyl}-3H-spiro[2-benzofuran-1,3′-pyrrolidin]-3-one;
(1R)-1′-(1-Naphthoyl)-3H-spiro[2-benzofuran-1,3′-pyrrolidin]-3-one;
(1R)-1′-(2-Naphthoyl)-3H-spiro[2-benzofuran-1,3′-pyrrolidin]-3-one;
(1R)-1′-(3,7-Dihydroxy-2-naphthoyl)-3H-spiro[2-benzofuran-1,3′-pyrrolidin]-3-one;
(1R)-1′-(6-Methoxy-1-naphthoyl)-3H-spiro[2-benzofuran-1,3′-pyrrolidin]-3-one;
(3′-Chloro-4′-{[(1R)-3-oxo-1′H,3H-spiro[2-benzofuran-1,3′-pyrrolidin]-1′-yl]carbonyl}biphenyl-3-yl)methyl dimethylcarbamate;
2-Methyl-3-{[(1R)-3-oxo-1′H,3H-spiro[2-benzofuran-1,3′-pyrrolidin]-1′-yl]carbonyl}phenyl acetate;
Methyl 4-(3-{[(1R)-3-oxo-1′H,3H-spiro[2-benzofuran-1,3′-pyrrolidin]-1′-yl]carbonyl}phenyl)piperidine-1-carboxylate;
tert-Butyl 4-(3-chloro-4-{[(1R)-3-oxo-1′H,3H-spiro[2-benzofuran-1,3′-pyrrolidin]-1′-yl]carbonyl}phenoxy)piperidine-1-carboxylate;
Methyl 4-(3-methyl-4-{[(1R)-3-oxo-1′H,3H-spiro[2-benzofuran-1,3′-pyrrolidin]-1′-yl]carbonyl}phenyl)-3,6-dihydropyridine-1 (2H)-carboxylate;
Methyl 4-(3-chloro-4-{[(1R)-3-oxo-1′H,3H-spiro[2-benzofuran-1,3′-pyrrolidin]-1′-yl]carbonyl}phenoxy)piperidine-1-carboxylate;
(1R)-1′-{2-Chloro-4-[5-(4-methylpiperazin-1-yl)pyridin-3-yl]benzoyl}-3H-spiro[2-benzofuran-1,3′-pyrrolidin]-3-one;
tert-Butyl 4-(4-methyl-3-{[(1R)-3-oxo-1′H,3H-spiro[2-benzofuran-1,3′-pyrrolidin]-1′-yl]carbonyl}phenyl)piperazine-1-carboxylate;
(1R)-1′-(2-Methyl-5-piperazin-1-ylbenzoyl)-3H-spiro[2-benzofuran-1,3′-pyrrolidin]-3-one;
Methyl 4-(4-methyl-3-{[(1R)-3-oxo-1′H,3H-spiro[2-benzofuran-1,3′-pyrrolidin]-1′-yl]carbonyl}phenyl)piperazine-1-carboxylate;
Ethyl 4-(4-methyl-3-{[(1R)-3-oxo-1′H,3H-spiro[2-benzofuran-1,3′-pyrrolidin]-1′-yl]carbonyl}phenyl)piperazine-1-carboxylate;
Propyl 4-(4-methyl-3-{[(1R)-3-oxo-1′H,3H-spiro[2-benzofuran-1,3′-pyrrolidin]-1′-yl]carbonyl}phenyl)piperazine-1-carboxylate;
Prop-2-yn-1-yl 4-(4-methyl-3-{[(1R)-3-oxo-1′H,3H-spiro[2-benzofuran-1,3′-pyrrolidin]-1′-yl]carbonyl}phenyl)piperazine-1-carboxylate;
Isopropyl 4-(4-methyl-3-{[(1R)-3-oxo-1′H,3H-spiro[2-benzofuran-1,3′-pyrrolidin]-1′-yl]carbonyl}phenyl)piperazine-1-carboxylate;
Isobutyl 4-(4-methyl-3-{[(1R)-3-oxo-1′H,3H-spiro[2-benzofuran-1,3′-pyrrolidin]-1′-yl]carbonyl}phenyl)piperazine-1-carboxylate;
(1R)-1′-{2-Methyl-5-[4-(methylsulfonyl)piperazin-1-yl]benzoyl}-3H-spiro[2-benzofuran-1,3′-pyrrolidin]-3-one;
(1R)-1′-{5-[4-(Ethylsulfonyl)piperazin-1-yl]-2-methylbenzoyl}-3H-spiro[2-benzofuran-1,3′-pyrrolidin]-3-one;
(1R)-1′-[5-(4-Acetylpiperazin-1-yl)-2-methylbenzoyl]-3H-spiro[2-benzofuran-1,3′-pyrrolidin]-3-one;
(1R)-1′-[2-Methyl-5-(4-propionylpiperazin-1-yl)benzoyl]-3H-spiro[2-benzofuran-1,3′-pyrrolidin]-3-one;
(1R)-1′-[5-(4-Isobutyrylpiperazin-1-yl)-2-methylbenzoyl]-3H-spiro[2-benzofuran-1,3′-pyrrolidin]-3-one;
(1R)-1′-{5-[4-(Cyclopropylcarbonyl)piperazin-1-yl]-2-methylbenzoyl}-3H-spiro[2-benzofuran-1,3′-pyrrolidin]-3-one;
tert-Butyl 4-(4-methyl-3-{[(1R)-3-oxo-1′H,3H-spiro[2-benzofuran-1,3′-pyrrolidin]-1′-yl]carbonyl}phenyl)-3,6-dihydropyridine-1 (2H)-carboxylate;
Methyl 4-(4-methyl-3-{[(1R)-3-oxo-1′H,3H-spiro[2-benzofuran-1,3′-pyrrolidin]-1′-yl]carbonyl}phenyl)-3,6-dihydropyridine-1 (2H)-carboxylate;
(1R)-1′-(2-Chloro-4-phenoxybenzoyl)-3H-spiro[2-benzofuran-1,3′-pyrrolidin]-3-one;
(1R)-1′-[2-Chloro-4-(1H-indol-6-yl)benzoyl]-3H-spiro[2-benzofuran-1,3′-pyrrolidin]-3-one;
(1R)-1′-[4-(6-aminopyridin-2-yl)-2-chlorobenzoyl]-3H-spiro[2-benzofuran-1,3′-pyrrolidin]-3-one;
N-[6-(3-Chloro-4-{[(1R)-3-oxo-1′H,3H-spiro[2-benzofuran-1,3′-pyrrolidin]-1′-yl]carbonyl}phenyl)pyridin-2-yl]acetamide;
N-[6-(3-Chloro-4-{[(1R)-3-oxo-1′H,3H-spiro[2-benzofuran-1,3′-pyrrolidin]-1′-yl]carbonyl}phenyl)pyridin-2-yl]-2-methylpropanamide;
N-[6-(3-Chloro-4-{[(1R)-3-oxo-1′H,3H-spiro[2-benzofuran-1,3′-pyrrolidin]-1′-yl]carbonyl}phenyl)pyridin-2-yl]cyclopropanecarboxamide;
N-[6-(3-Chloro-4-{[(1R)-3-oxo-1′H,3H-spiro[2-benzofuran-1,3′-pyrrolidin]-1′-yl]carbonyl}phenyl)pyridin-2-yl]ethanesulfonamide;
N-[6-(3-Chloro-4-{[(1R)-3-oxo-1′H,3H-spiro[2-benzofuran-1,3′-pyrrolidin]-1′-yl]carbonyl}phenyl)pyridin-2-yl]butanamide;
Methyl [6-(3-chloro-4-{[(1R)-3-oxo-1′H,3H-spiro[2-benzofuran-1,3′-pyrrolidin]-1′-yl]carbonyl}phenyl)pyridin-2-yl]carbamate;
Ethyl [6-(3-chloro-4-{[(1R)-3-oxo-1′H,3H-spiro[2-benzofuran-1,3′-pyrrolidin]-1′-yl]carbonyl}phenyl)pyridin-2-yl]carbamate;
Propyl [6-(3-chloro-4-{[(1R)-3-oxo-1′H,3H-spiro[2-benzofuran-1,3′-pyrrolidin]-1′-yl]carbonyl}phenyl)pyridin-2-yl]carbamate;
Isopropyl [6-(3-chloro-4-{[(1R)-3-oxo-1′H,3H-spiro[2-benzofuran-1,3′-pyrrolidin]-1′-yl]carbonyl}phenyl)pyridin-2-yl]carbamate;
Isobutyl [6-(3-chloro-4-{[(1R)-3-oxo-1′H,3H-spiro[2-benzofuran-1,3′-pyrrolidin]-1′-yl]carbonyl}phenyl)pyridin-2-yl]carbamate;
(1R)-1′-[2-Chloro-4-(pyridin-3-yloxy)benzoyl]-3H-spiro[2-benzofuran-1,3′-pyrrolidin]-3-one;
(1R)-1′-(2-Chloro-4-quinolin-7-ylbenzoyl)-3H-spiro[2-benzofuran-1,3′-pyrrolidin]-3-one;
5-(3-Chloro-4-{[(1R)-3-oxo-1′H,3H-spiro[2-benzofuran-1,3′-pyrrolidin]-1′-yl]carbonyl}phenyl)-N-cyclopropylpyridine-2-carboxamide;
(1R)-1′-[4-(4-Hydroxyphenoxy)benzoyl]-3H-spiro[2-benzofuran-1,3′-pyrrolidin]-3-one;
5-(3-Chloro-4-{[(1R)-3-oxo-1′H,3H-spiro[2-benzofuran-1,3′-pyrrolidin]-1′-yl]carbonyl}phenyl)-N-ethylpyridine-2-carboxamide;
5-(3-Chloro-4-{[(1R)-3-oxo-1′H,3H-spiro[2-benzofuran-1,3′-pyrrolidin]-1′-yl]carbonyl}phenyl)-N,N-diethylpyridine-2-carboxamide;
5-(3-Chloro-4-{[(1R)-3-oxo-1′H,3H-spiro[2-benzofuran-1,3′-pyrrolidin]-1′-yl]carbonyl}phenyl)-N-cyclopropylpyridine-2-carboxamide;
(1R)-1′-{4-[6-(Azetidin-1-ylcarbonyl)pyridin-3-yl]-2-chlorobenzoyl}-3H-spiro[2-benzofuran-1,3′-pyrrolidin]-3-one;
5-(3-Chloro-4-{[(1R)-3-oxo-1′H,3H-spiro[2-benzofuran-1,3′-pyrrolidin]-1′-yl]carbonyl}phenyl)-N-methylpyridine-2-carboxamide;
5-(3-Chloro-4-{[(1R)-3-oxo-1′H,3H-spiro[2-benzofuran-1,3′-pyrrolidin]-1′-yl]carbonyl}phenyl)-N,N-dimethylpyridine-2-carboxamide;
(1R)-1′-{2-Chloro-4-[(6-methylpyridin-3-yl)oxy]benzoyl}-3H-spiro[2-benzofuran-1,3′-pyrrolidin]-3-one;
6-(3-Chloro-4-{[(1R)-3-oxo-1′H,3H-spiro[2-benzofuran-1,3′-pyrrolidin]-1′-yl]carbonyl}phenyl)-N-methylpyridine-2-carboxamide;
6-(3-Chloro-4-{[(1R)-3-oxo-1′H,3H-spiro[2-benzofuran-1,3′-pyrrolidin]-1′-yl]carbonyl}phenyl)-N,N-dimethylpyridine-2-carboxamide;
6-(3-Chloro-4-{[(1R)-3-oxo-1′H,3H-spiro[2-benzofuran-1,3′-pyrrolidin]-1′-yl]carbonyl}phenyl)-N-ethylpyridine-2-carboxamide;
6-(3-Chloro-4-{[(1R)-3-oxo-1′H,3H-spiro[2-benzofuran-1,3′-pyrrolidin]-1′-yl]carbonyl}phenyl)-N,N-diethylpyridine-2-carboxamide;
6-(3-Chloro-4-{[(1R)-3-oxo-1′H,3H-spiro[2-benzofuran-1,3′-pyrrolidin]-1′-yl]carbonyl}phenyl)-N-cyclopropylpyridine-2-carboxamide;
(1R)-1′-{4-[6-(Azetidin-1-ylcarbonyl)pyridin-2-yl]-2-chlorobenzoyl}-3H-spiro[2-benzofuran-1,3′-pyrrolidin]-3-one;
(1R)-1′-{2-Chloro-4-[(6-methylpyridin-2-yl)oxy]benzoyl}-3H-spiro[2-benzofuran-1,3′-pyrrolidin]-3-one;
(1R)-1′-[4-(3-Hydroxyphenoxy)benzoyl]-3H-spiro[2-benzofuran-1,3′-pyrrolidin]-3-one;
(1R)-1′-{2-Chloro-4-[(2-methylpyridin-3-yl)oxy]benzoyl}-3H-spiro[2-benzofuran-1,3′-pyrrolidin]-3-one;
(1R)-1′-{2-Chloro-4-[(2,6-dimethylpyridin-4-yl)oxy]benzoyl}-3H-spiro[2-benzofuran-1,3′-pyrrolidin]-3-one;
6-(3-Chloro-4-{[(1R)-3-oxo-1′H,3H-spiro[2-benzofuran-1,3′-pyrrolidin]-1′-yl]carbonyl}phenoxy)-N-methylnicotinamide;
6-(3-Chloro-4-{[(1R)-3-oxo-1′H,3H-spiro[2-benzofuran-1,3′-pyrrolidin]-1′-yl]carbonyl}phenoxy)-N,N-diethylnicotinamide;
(1R)-1′-(4-{[3-Chloro-5-(trifluoromethyl)pyridin-2-yl]oxy}benzoyl)-3H-spiro[2-benzofuran-1,3′-pyrrolidin]-3-one;
5-(4-Chloro-3-{[(1R)-3-oxo-1′H,3H-spiro[2-benzofuran-1,3′-pyrrolidin]-1′-yl]carbonyl}phenyl)-N-methylpyridine-2-carboxamide;
5-(4-Chloro-3-{[(1R)-3-oxo-1′H,3H-spiro[2-benzofuran-1,3′-pyrrolidin]-1′-yl]carbonyl}phenyl)-N,N-dimethylpyridine-2-carboxamide;
5-(4-Chloro-3-{[(1R)-3-oxo-1′H,3H-spiro[2-benzofuran-1,3′-pyrrolidin]-1′-yl]carbonyl}phenyl)-N-ethylpyridine-2-carboxamide;
5-(4-Chloro-3-{[(1R)-3-oxo-1′H,3H-spiro[2-benzofuran-1,3′-pyrrolidin]-1′-yl]carbonyl}phenyl)-N,N-diethylpyridine-2-carboxamide; and
(1R)-1′-{4-[(6-Methylpyridazin-3-yl)oxy]benzoyl}-3H-spiro[2-benzofuran-1,3′-pyrrolidin]-3-one, or a pharmaceutically acceptable salt thereof.

44. A composition comprising a compound of claim 1 and a pharmaceutically acceptable carrier.

45. A method of treating a disease in a patient, wherein said disease is associated with expression or activity of 11βHSD1 or expression or activity MR, comprising administering to said patient a therapeutically effective amount of with a compound of Formula Ia or Ib: 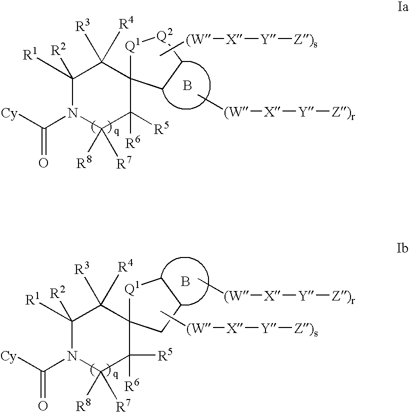
or pharmaceutically acceptable salt or prodrug thereof, wherein:
Cy is aryl, heteroaryl, cycloalkyl, or heterocycloalkyl, each optionally substituted by 1, 2, 3, 4 or 5 —U-T-W—X—Y-Z;
Q¹ is O, S, NH, CH₂, CO, CS, SO, SO₂, OCH₂, SCH₂, NHCH₂, CH₂CH₂, COCH₂, CONH, COO, SOCH₂, SONH, SO₂CH₂, or SO₂NH;
Q² is O, S, NH, CH₂, CO, CS, SO, SO₂, OCH₂, SCH₂, NHCH₂, CH₂CH₂, COCH₂, CONH, COO, SOCH₂, SONH, SO₂CH₂, or SO₂NH;
ring B is an aryl, heteroaryl, cycloalkyl, or heterocycloalkyl group fused with the ring containing Q¹ and Q²;
R¹, R², R³, R⁴, R⁵, R⁶, R⁷, and R⁸ are each, independently, H or —W′—X′—Y′-Z′;
or R¹ and R² together with the C atom to which they are attached form a 3-20 membered cycloalkyl group or a 3-20 membered heterocycloalkyl group optionally substituted by 1 or 2 —W″—X″—Y″-Z″;
or R³ and R⁴ together with the C atom to which they are attached form a 3-20 membered cycloalkyl group or a 3-20 membered heterocycloalkyl group optionally substituted by 1 or 2 —W″—X″—Y″-Z″;
or R and R⁶ together with the C atom to which they are attached form a 3-20 membered cycloalkyl group or a 3-20 membered heterocycloalkyl group optionally substituted by 1 or 2 —W″—X″—Y″-Z″;
or R⁷ and R⁸ together with the C atom to which they are attached form a 3-20 membered cycloalkyl group or a 3-20 membered heterocycloalkyl group optionally substituted by 1 or 2 —W″—X″—Y″-Z″;
or R¹ and R⁵ together form an C₁₋₄ alkylene bridge optionally substituted by 1 or 2 or R³ and R⁵ together form an C₁₋₄ alkylene bridge optionally substituted by 1 or 2 —W″—X″—Y″-Z″;
U is absent, C₁₋₆alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;
T is absent, C₁₋₆ alkylenyl, C₂₋₆ alkenylenyl, C₂₋₆ alkynylenyl, aryl, aryloxy, cycloalkyl, heteroaryl, heteroaryloxy, or heterocycloalkyl, wherein said C₁₋₆ alkylenyl, C₂₋₆ alkenylenyl, C₂₋₆ alkynylenyl, cycloalkyl, heteroaryl or heterocycloalkyl is optionally substituted by one or more halo, CN, NO₂, OH, C₁₋₄ alkoxy, C₁₋₄ haloalkoxy, amino, C₁₋₄ alkylamino or C₂₋₈ dialkylamino;
W, W′ and W″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆ alkenylenyl, C₂₋₆ alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;
X, X′ and X″ are each, independently, absent, C₁₋₆ alkylenyl, C₂₋₆ alkenylenyl, C₂₋₆ alkynylenyl, aryl, cycloalkyl, heteroaryl or heterocycloalkyl, wherein said C₁₋₆ alkylenyl, C₂₋₆ alkenylenyl, C₂₋₆ alkynylenyl, cycloalkyl, heteroaryl or heterocycloalkyl is optionally substituted by one or more halo, CN, NO₂, OH, C₁₋₄ alkoxy, C₁₋₄ haloalkoxy, amino, C₁₋₄ alkylamino or C₂₋₈ dialkylamino;
Y, Y′ and Y″ are each, independently, absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein said C₁₋₆ alkylenyl, C₂₋₆alkenylenyl, C₂₋₆alkynylenyl are each optionally substituted by 1, 2 or 3 halo, OH, C₁₋₄ alkoxy, C₁₋₄ haloalkoxy, amino, C₁₋₄ alkylamino or C₂₋₈ dialkylamino;
Z, Z′ and Z″ are each, independently, H, halo, CN, NO₂, OH, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, aryl, cycloalkyl, heteroaryl or heterocycloalkyl, wherein each of said C₁₋₆ alkyl, C₂₋₆ alkenyl, C₂₋₆ alkynyl, aryl, cycloalkyl, heteroaryl or heterocycloalkyl is optionally substituted by 1, 2 or 3 halo, C₁₋₆ alkyl, C₁₋₆ hydroxyalkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR, OC(O)R^{b}, OC(O)NR^{c}R^{d}, —C₁₋₄ alkyl-OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, NR^{c}S(O)₂R^{b} or S(O)₂NR^{c}R^{d};
wherein two —W—X—Y-Z together with the atom to which they are both attached optionally form a 3-20 membered cycloalkyl group or 3-20 membered heterocycloalkyl group optionally substituted by 1, 2 or 3 —W″—X″—Y″-Z″;
wherein two —W′—X′—Y′-Z′ together with the atom to which they are both attached optionally form a 3-20 membered cycloalkyl group or 3-20 membered heterocycloalkyl group optionally substituted by 1, 2 or 3 —W″—X″—Y″-Z″;
wherein —W—X—Y-Z is other than H;
wherein —W′—X′—Y′-Z′ is other than H;
wherein —W″—X″—Y″-Z″ is other than H;
R^{a} is H, C₁₋₆ alkyl, C₁₋₆ haloalkyl, C₂₋₆ alkenyl, C₂₋₆ alkynyl, aryl, cycloalkyl, heteroaryl or heterocycloalkyl;
R^{b} is H, C₁₋₆ alkyl, C₁₋₆ haloalkyl, C₂₋₆ alkenyl, C₂₋₆ alkynyl, aryl, cycloalkyl, heteroaryl or heterocycloalkyl;
R^{c} is H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
or R^{c} and R^{d} together with the N atom to which they are attached form a 4-, 5-, 6- or 7-membered heterocycloalkyl group;
R^{e} and R^{f} are each, independently, H, C₁₋₆ alkyl, C₁₋₆ haloalkyl, C₂₋₆ alkenyl, C₂₋₆ alkynyl, aryl, cycloalkyl, arylalkyl, or cycloalkylalkyl;
or R^{e} and R^{f} together with the N atom to which they are attached form a 4-, 5-, 6- or 7-membered heterocycloalkyl group;
q is 0, 1, or 2;
r is 0, 1 or 2; and
s is 0, 1 or 2;
with the proviso that when the compound has Formula Ia, Q¹ is CH₂, Q² is CH₂, and q is 1, then r is 1 or 2; and with the proviso that Cy is other than cyclopropyl substituted by 1 or 2 —U-T-W—X—Y-Z.

46. The method of claim 45 wherein said disease is obesity, diabetes, glucose intolerance, insulin resistance, hyperglycemia, hypertension, hyperlipidemia, cognitive impairment, depression, dementia, glaucoma, cardiovascular disorders, osteoporosis, inflammation, a cardiovascular, renal or inflammatory disease, heart failure, atherosclerosis, arteriosclerosis, coronary artery disease, thrombosis, angina, peripheral vascular disease, vascular wall damage, stroke, dyslipidemia, hyperlipoproteinaemia, diabetic dyslipidemia, mixed dyslipidemia, hypercholesterolemia, hypertriglyceridemia, metabolic syndrome or general aldosterone-related target organ damage.
